(19) United States
(12) Patent Application Publication
(10) Pub. No.: US 2026/0218121 A1
(43) Pub. Date: Jul. 30, 2026

(54) METHODS FOR CELL SURFACE REMODELING

(71) Applicants: NANTES UNIVERSITÉ, NANTES CEDEX 1 (FR); INSTITUT NATIONAL DE LA SANTÉ ET DE LA RECHERCHE MÉDICALE, PARIS (FR); CENTRE NATIONAL DE LA RECHERCHE SCIENTIFIQUE, PARIS (FR)

(72) Inventors: SÉBASTIEN GOUIN, THOUARÉ SUR LOIRE (FR); DAVID DENIAUD, SAINT HERBLAIN (FR); MATHIEU MEVEL, NANTES (FR); SÉBASTIEN DEPIENNE, NANTES (FR); DIMITRI ALVAREZ-DORTA, NANTES (FR); MOHAMMED BOUZELHA, NANTES (FR)

(21) Appl. No.: 19/146,994

(22) PCT Filed: Jan. 11, 2024
(86) PCT No.: PCT/EP2024/050599

§ 371 Date: Jul. 10, 2025

(30) Foreign Application Priority Data
Jan. 12, 2023 (EP) 23305040.0

(51) Int. Cl. C12N 5/00

(52) U.S. Cl. CPC C12N 5/0006

(57) ABSTRACT

The Invention relates to an in vitro method for chemically-modifying the surface of a cell, which comprises incubating said cell with N-substituted luminol moiety or a N-substituted phenylurazole in conditions conducive for reacting said chemical reagent with a cell surface component so as to form a covalent bond. The Invention also relates to a cell obtainable or obtained by such a process and its use in therapeutic, diagnostic or research field.

### FIELD OF THE INVENTION

The invention relates to methods for chemically modifying cell surface.

### BACKGROUND OF THE INVENTION

The cell surface-associated proteins and carbohydrates play a pivotal role in many biological events including intercellular communications and binding, signal transduction, and host-pathogens interactions. A deeper understanding of these biological processes relies on the ability to pattern cell surfaces with specific probes, drugs, binding proteins or carbohydrates.

The modification of cell membranes to introduce non-native chemical species have been already investigated by several scientific teams and finds a wide range of therapeutic and research applications.

The modification of bacterial cell surfaces has been studied for both diagnostic and therapeutic purposes. Research studies using living bacteria for treating solid tumors has seen considerable progress in recent years (Zhou et al., Nat. Rev. Cancer, 2018, 18, 727-743). Conventional chemotherapeutics show low penetration and limited accumulation in poorly vascularized hypoxic areas of tumours. By contrast, anaerobic bacteria easily colonize and are able to proliferate in those necrotic and deep regions. It was thus suggest using anaerobic bacteria to initiate anti-tumor immune response. This bacterial-based anticancer therapy can be significantly improved by the chemical modification of bacterial surface with immunity check point inhibitors, chemotherapeutic drugs, tumor-specific antigens, and photothermal sensitizers (Cao et al., J. Controlled Release, 2020, 326, 396-407, Gupta et al., Vaccines, 2021, 9, 1497).

Chemical modification of eukaryotic cell surfaces is also a rapidly evolving research field with strong potential in drug delivery, cell-based therapies or tissue engineering (Bi et al., Chem-Eur. J. 2018, 24, 8042-8050, Kellam et al., Chem. Soc. Rev., 2003, 32, 327-337). For instance, cell surface remodelling may be used to i) improve targeting as illustrated on mesenchymal stem cells carrying modified sugar coat with E-selectin ligands for enhanced bone tropism (Sackstein et al. Nat. Med., 2008, 14, 181-187) or ii) mask selective antigens, as showed with the PEGylation of donor red blood cells in chronic transfusions to avoid immune rejections (Scott et al., PNAS, 1997, 94, 7566-7571).

As of today, there are few strategies for modifying cell surface. The main strategy for introducing a non-natural moiety on surface cell is metabolic engineering. This two-step method relies on the use of foreign synthetic substrates that are recognized and processed by the cell biosynthesis enzymatic machinery for incorporation into the cell-surface components. The synthetic substrate bears a chemical reactive moiety able to specifically react through bio-orthogonal reaction, e.g. click chemistry, enabling the covalent grafting of the non-natural moiety of interest, in a subsequent step. Most of the time, the synthetic substrate is a non-natural, synthetic sugar but strategies based on unnatural amino acid (UAA) residue has been developed as well, but requires the use of recombinant cell or specific strain, e.g. the methionine auxotrophic E. coli which is unable to synthetize the native amino acid substrate but can recognize and use analogues such as azidohomoalanine during translation

Metabolic engineering is a powerful technique for decorating both mammalian and bacterial cell surface. However, there is a need for the development of direct and faster labelling methods which would avoid the cell culture step required to incorporate the synthetic substrate, in particular for bacterial or cell lines with a low propensity to incorporate synthetic sugar precursors in their glycocalyx.

Consequently, direct chemical modification of the constituents of cell membrane seems to be a technique of interest as it does not require any genetic manipulation or pre-culture step. This strategy has been investigated for a long time, but remains poorly exemplified as of today.

Indeed, the direct chemical modification of cell surface is a real challenge. The cell surface is a highly heterogeneous environment with proteins, carbohydrates, lipids and other molecules controlling vital cell function, which renders quite complex the decoration with non-native chemical molecules on specific site (in particular on membrane protein) and in a controlled manner. The challenge is further complicated by the need to ensure that the chemical modification would not impair the cell viability and would not induce undesirable changes in the pattern of cell behavior.

There are some reports concerning the functionalization of the surfaces of bacterial and mammalian cells by direct chemical modification by using electrophilic anchors which react with amino and thiol groups from the side-chain of lysine and cysteine, e.g. with anchors containing maleimide, cyanuric chloride-activated moiety or N-hydroxy-succinimide esters. Such strategies enabled to functionalize the cell surface with different molecules such as DNA, PEG, nanoparticles or affinity tag e.g. biotin which can be in turn modified with streptavidin bearing a molecule of interest (Bi, supra, Kellam, supra). However, thiol- and amino-based chemistry suffers from a lack of site-specificity and is not suitable for coupling complex molecules comprising nucleophilic groups (e.g. thiol, amine, hydroxyl) due to possible side-reactions with the reactive moieties used in this kind of chemistry with these nucleophilic groups.

Methods targeting less nucleophilic and/or less abundant amino acid such as methionine, guanidine or tyrosine are poorly investigated.

Thus, there is still a need for new methods for directly cell surface remodelling.

### SUMMARY OF THE INVENTION

The Invention relates to an in vitro method for chemically-modifying the surface of a cellular entity, which comprises incubating said cellular entity with a chemical reagent bearing a N-substituted luminol moiety or a N-substituted phenyl-urazole in conditions conducive for reacting said chemical reagent with a cell surface component so as to form a covalent bound. The cellular entity is preferably selected from procaryotes, eucaryotic cells and extracellular vesicles thereof. Preferably, the cellular entity is a cell such as a mammal cell or a bacteria.

The method is preferably performed by electrochemistry and comprises the step of incubating the cellular entity (preferably the cell) with a chemical reagent of formula (I) or (IP):

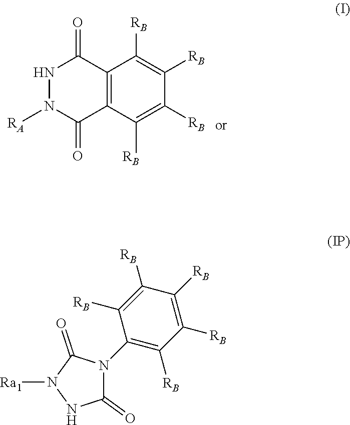

wherein:
R_{A} is a C₁-C₆ alkyl, a C₆-C₁₄ aryl optionally substituted, a (C₆-C₁₄ aryl)-(C₁-C₃ alkyl) optionally substituted, or -(Y₁)ₙ-M₁
Ra₁ is H or R_{A},
Each R_{B} is independently a group of formula -(Y)ₙ-M₁, a hydrogen or a substituent chosen from a halogen, C₁-C₆ alkyl, C₆-C₁₄ aryl, C₃-C₆ cycloalkyl, C₁-C₆ alkoxy, C₁-C₆ alkylamino, C₂-C₆ heterocycle, C₁-C₆ alkanoyl, C₁-C₆ carboxy esters, C₁-C₆ acylamino, —COOH, —CONH₂, —NO₂, —SO₃H, —CN, —CF₃, C₁-C₆ hydroxyalkyl, C₁-C₆ haloalkyl, C₁-C₆ alkylthio, C₁-C₆ thioalkyl, C₂-C₁₀ alkoxyalkyl, or C₂-C₆ alkoxycarbonyloxy,
n is 0 or 1,
Y₁ is a spacer,
M₁ is a functional moiety and
at least one group among R_{A} and R_{B} groups is -(Y₁)ₙ-M₁ in formula (I) or at least one group among Rₐ₁ and R_{B} groups is -(Y₁)ₙ-M₁ in formula (IP)
   in the presence of a potential difference enabling the electro-activation of said chemical reagent of formula (I) or (IP) into an oxidized form able to react with a tyrosine residue present in a surface component so as to obtain at least one chemically-modified tyrosine residue of formula (B) or (BP) on the surface of the cellular entity, respectively:

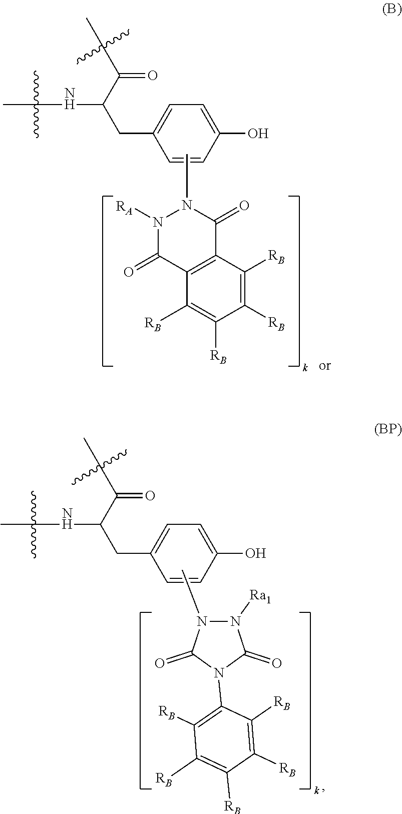

wherein:
R_{A}, Ra₁, and R_{B} are as defined in formula (I) and (IP),
k is 1 or 2.

In some embodiments R_{A} is selected from methyl, phenyl or benzyl, preferably methyl and Rₐ₁ is selected from C₁-C₆ alkyl such as methyl and H. In such embodiments, at least one R_{B} is -(Y₁)ₙ-M₁.

In some embodiments, the method of the Invention is such that:
M₁ comprises or consists of a moiety selected from the group consisting of a chemical reactive group enabling biorthogonal reaction such as a click-chemistry reactive group, a targeting agent, a steric shielding agent, a labelling agent, an oligonucleotide, a drug, a nanoparticle including a liposome, a ligand e.g. a cell-type ligand, a polypeptide, peptide, a hormone, a polysaccharide, and combinations thereof, and/or
Y₁ is a chemical chain group comprising from 2 to 500 carbon atoms and selected from the group consisting of polymers including homopolymers, copolymers and block polymers, peptides, oligosaccharides, and saturated or unsaturated hydrocarbon chains optionally interrupted by one or several heteroatoms and/or by one or several cyclic or heterocyclic moieties, optionally having an heteroatom, such as S, O and NH, at least one of its extremity, and optionally substituted by one or several substituents, and combinations thereof.

In some embodiments, the chemical reagent is of formula (I), preferably of formula (I-c):

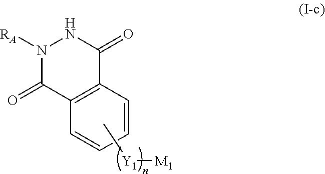

Wherein R_{A} is C₁-C₃ alkyl, a phenyl optionally substituted, or a benzyl optionally substituted, more preferably a methyl, a phenyl or a benzyl and even more preferably a methyl.

In some embodiments, the method of the invention may be performed in an electrochemical system with three electrodes comprising a working electrode, a counter-electrode and a reference electrode by applying a constant potential difference between the working electrode and the reference electrode, the potential difference being preferably the potential oxidation of the chemical reagent±200 mV.

In some embodiments, the cellular entity is a cell selected from:
bacteria, in particular from E. coli
mammalian cells, in particular human cells such as cell lines or such as cells isolated from subjects, preferably selected from erythrocytes, Hematopoietic Stem cells (HSC), Peripheral Blood Mononuclear cells (PBMC) and in particular T lymphocytes and dendritic cells (DC).
   with proviso that the cell is not a human embryo or is not obtained by a method resulting in destruction of human embryo.

The Invention also relates to a method for decorating the surface of a cellular entity, preferably a cell, with a functional moiety M₂, which comprises:
(i) a step of chemically modifying the surface of the cellular entity according to the method as described above, wherein M₁ is a click chemistry reactive group
(ii) a step of immobilizing M₂ at the cellular entity surface by promoting a click reaction with M₁, preferably by incubating the cellular entity obtained in step (i) with a compound of formula (III),

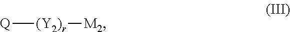

wherein:
Q is a click-chemistry group that is able to react with M₁ through a click chemistry reaction,
r is 0 or 1,
Y₂ is a spacer, and
M₂ is the functional group to be immobilized on the surface of the cellular entity.
   in conditions conducive to the reaction of Q with M₁.

Preferably, M₁ is an azido and Q is a strained-alkyne, the reaction being a strain promoted alkyne-azido cycloaddition (SPAAC).

The Invention further relates to a method for decorating the surface of a cellular entity with a functional moiety M₂, which comprises:

(i) a step of chemically modifying the surface of the cellular entity according to the method as described above,
(ii) a step of incubating the cellular entity obtained in step (i) with a compound of formula (III),

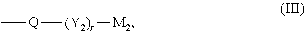

wherein:
r is 0 or 1,
Y₂ is a spacer, and
M₂ is the functional group to be immobilized on the surface of the cellular entity, and
Q and M₁ are selected so as to specifically interact together and form a stable complex, for example M₁ is biotin and Q is streptavidin or avidin.
   in conditions conducive to enable the formation of a complex between Q and M₁.

In an additional aspect, the Invention relates to a cellular entity, preferably a cell obtainable or obtained by a method as defined above.

The Invention further relates to a cellular entity, preferably a cell, having at least one chemically-modified tyrosine residue present in a cell surface component, preferably in a cell surface protein, which is of formula (C) or (CP):

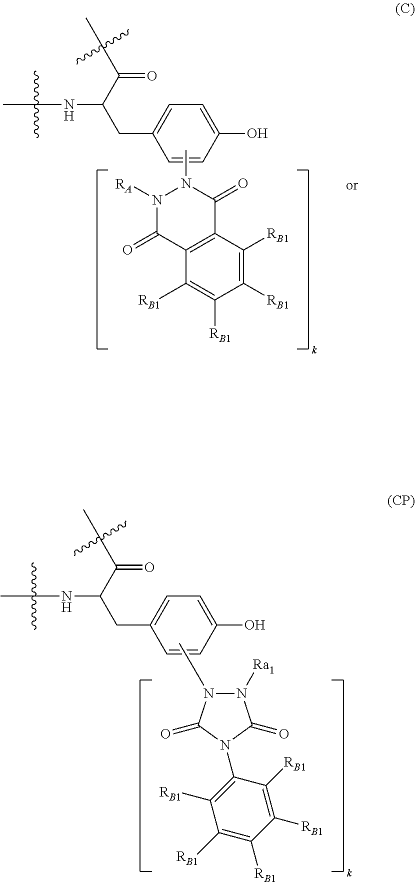

or, preferably of formula (C-1) or (C-2) as followed:

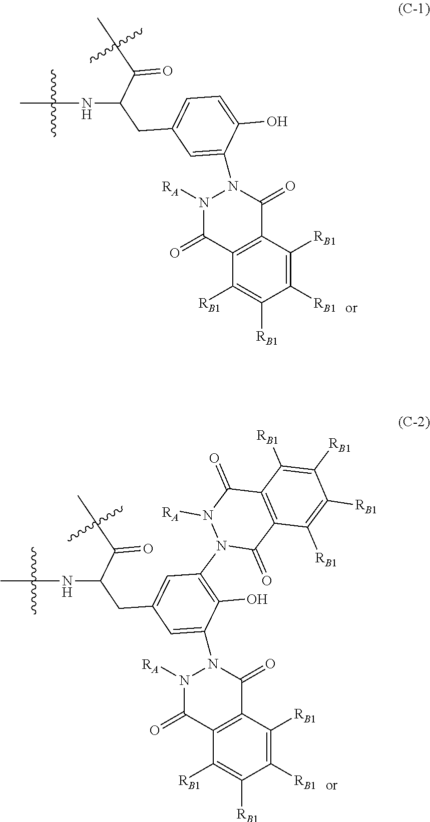

wherein:
k is 1 or 2,
R_{A} is selected from the group consisting of C₁-C₃ alkyl, a phenyl optionally substituted, or a benzyl optionally substituted, and -(Y)ₙ-M
Ra₁ is R_{A} or H,
each R_{B1} is independently a group of formula -(Y)ₙ-M, a hydrogen or a substituent selected from the group consisting of a halogen, C₁-C₆ alkyl, C₆-C₁₄ aryl, C₃-C₆ cycloalkyl, C₁-C₆ alkoxy, C₁-C₆ alkylamino, C₂-C₆ heterocycle, C₁-C₆ alkanoyl, C₁-C₆ carboxy esters, C₁-C₆ acylamino, —COOH, —CONH₂, —NO₂, —SO₃H, —CN, —CF₃, C₁-C₆ hydroxyalkyl, C₁-C₆ haloalkyl, C₁-C₆ thioalkyl, C₁-C₆ alkylthio, C₂-C₁₀ alkoxyalkyl, and C₂-C₆ alkoxycarbonyloxy,
n is 0 or 1,
Y is a spacer,
M is a functional moiety and
At least one group among R_{A} and R_{B} groups is -(Y)ₙ-M in formula (C), (C-1) or (C-2) or at least one group among Rₐ₁ and R_{B} groups is -(Y)ₙ-M in formula (CP)

In some embodiments, the cellular entity is a cell. The cell can be characterized by one or several specific features:
all R_{B1} groups are all H except one R_{B1} which is a group of formula -(Y)ₙ-M, and/or
R_{A} is C₁-C₃ alkyl, a phenyl optionally substituted, or a benzyl optionally substituted, more a methyl, a phenyl or a benzyl and even more preferably a methyl, and/or
M is selected from the group consisting of a click chemistry reactive group, preferably N₃, tetrazine and cyclooctyne, a cytotoxic or antitumoral drug preferably doxorubicin and monomethylauristatin E, a labelling agent preferably complexes of radionuclides such as 67Cu in DOTA and fluorophores such as fluorescein and derivatives thereof such as FITC, or Cyanines 3/5/7 (Cy3, Cy5, Cy7), a shielding or masking agent such as PEG, carbohydrate antigens and oligo- or polysaccharide ligands such as oligo-mannosides or sialosides, protein tags such as biotin/strep(avidin), polypeptides comprising an antigen-binding domain such as VHH and/or
the cell is selected from the group consisting of bacteria, erythrocytes, Hematopoietic Stem cells (HSC), Peripheral Blood Mononuclear cells (PBMC) and in particular T lymphocytes and dendritic cells (DC).
   The Invention further relates to the use of the cellular entity as defined above in therapy, e.g. as drug carrier, in diagnostic, e.g. as imaging agent in vivo or ex vivo or as a research tool.
   The Invention further relates to the use of a cellular entity, preferably a cell as defined herein as a research tool in vitro or as an imaging agent in vitro.
   The Invention also relates to the use of a cellular entity, preferably a cell as defined herein in the manufacture of an in vivo diagnostic agent or a medicine.
   In an additional aspect, the Invention relates to the use of a compound of formula (I) or (IP):

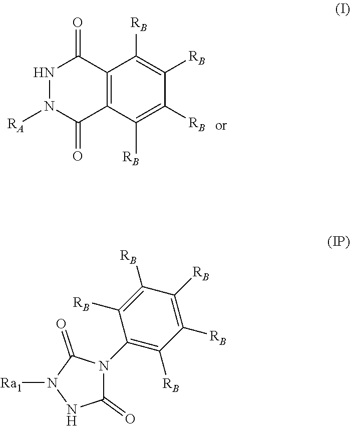

as defined in herein as an agent for chemically modifying the surface of a cellular entity, preferably a cell, by electrochemical bioconjugation.

Preferably, the compound is of formula (I) and is such that R_{A} is a C₁-C₆ alkyl and at least one R_{B} is of formula -(Y)ₙ-M and the other R_{B} are H.

### FIGURES

FIG. 1 shows experimental setup and electrochemical cell assembled for cells electro-bioconjugation. Anode, cathode and reference electrodes are clipped to the electrodes holder (A) and dipped into a low binding vial filled with cells and N-methyl luminol derivative solution (B) inserted into the 5 mL glass vial to form (ABC). Alligator clips connect electrical connections from (A) to potentiostat. The latter is USB-controlled by a computer (software EC-Lab).

FIG. 2A shows cyclic voltammetry of Methyl luminol azido derivative (NMeLum-N₃) at scan rates 25, 50, 75 and 100 mV/s highlighting a coherent electrochemical process controlled by diffusion. FIG. 2B shows multicyclic voltammetry (n=6) of NMeLum-N₃ at 100 mV/s outlining a clean reversible oxidation process.

FIG. 3 shows growth curves of electro-conjugated E. Coli with NMeLum-N₃ (1 h, 1 mM conc.) as compared to untreated E. Coli strain. Each sample was cultured (n=3) and OD₆₀₀ was regularly measured.

FIG. 4 shows optical (A) and fluorescence (B) microscopy images of Staph. Epidermidis subjected to incubation with fluorescent cyclooctyne DBCO-PEG4-CR110 probe (1 h, 0.1 mM final conc.) only; optical (C) and fluorescence (D) microscopy images of Staph. Epidermidis subjected to electro-conjugation with NMeLum-N₃ (15 min, 1 mM conc.) followed by incubation with fluorescent cyclooctyne DBCO-PEG4-CR110 probe (1 h, 0.1 mM final conc.).

FIG. 5 shows mean fluorescence intensity measured by flow cytometry (n=3) for unmodified bacteria (E. coli or S. epidermidis), bacteria incubated only with fluorescent cyclooctyne DBCO-PEG4-CR110 probe (1 h, 0.1 mM final conc., only SPAAC), and electro-bioconjugation experiments with NMeLum-N₃ for 15-, 30- or 60 min (1 mM conc., n=3) followed by incubation with fluorescent cyclooctyne DBCO-PEG4-CR110 probe (1 h, 0.1 mM final conc.).

FIG. 6 shows SDS-PAGE electrophoresis on gels of non-fractionated (total), cytosol and membrane proteins fractions after bacterial lysis and fractionation protocol applied to E. Coli strains after 1 h electro-conjugation with NMeLum-N₃ (1 mM final conc.) followed by 1 h SPAAC with DBCO-PEG4-CR110 probe (0.1 mM final conc.). Gels were colored by Commassie Brilliant Blue (left) to confirm presence of all proteins in the fractions, and fluorescence detection at 490 nm (right) revealed fluorescent proteins in total and membrane lanes only, standing for a membrane electro-labelling.

FIG. 7 shows cells % viability evaluation using Trypan Blue and Zombie Yellow assays for both HEK293 and HeLa cells subjected to different treatment (control, washings, cells incubated with NMeLum-N₃ (1 mM final conc.) during 30 min without voltage followed by SPAAC with DBCO-PEG4-FAM (0.2 mM final conc) (30′ eY-click (OFF)), and cells incubated with NMeLum-N₃ (1 mM final conc.) during 30 min under effective electro-bioconjugation experiment (i.e. 30 min with voltage) followed by SPAAC with DBCO-PEG4-FAM (0.2 mM final conc) (30′ eY-click (ON). For Trypan Blue assay, % of viable cells is calculated from the ratio of positive viable cells and total cells obtained from Vi-CELL XR. For Zombie Yellow, % viable cells is calculated from the formula: % viability=100%−(% fluorescent cells measured by flow cytometry after cells incubation with zombie yellow).

FIG. 8 shows mean fluorescence intensity measured by flow cytometry (n=3) for unmodified HEK293 cells, cells incubated only with fluorescent cyclooctyne DBCO-PEG4-FAM (0.2 mM final conc) (Only SPAAC), cells incubated with NMeLum-N₃ (1 mM final conc.) during 30 min without voltage (30′ OFF+SPAAC) and effective electro-bioconjugation experiments with NMeLum-N₃ (1 mM final conc.) followed by incubation with fluorescent cyclooctyne DBCO-PEG4-FAM (0.2 mM final conc) (30′ ON+SPAAC) (n=3).

FIG. 9 shows confocal microscopy images of NMeLum-N₃ (1 mM final conc.) electro-conjugated HEK293 cells subjected to subsequent incubation with fluorescent cyclooctyne DBCO-PEG4-FAM (FITC), an AF647-labelled WGA lectin (WGA) and (DAPI). The (Merge) image outlines unambiguous overlapping signals for FITC and WGA, standing for membrane labelling during electro-conjugation.

FIG. 10 shows viability (n=3) (FIG. 10A) and mean fluorescence intensity measured by flow cytometry (n=3) (FIG. 10B) for (i) unmodified DC2.4 cells, (ii) cells incubated with 50 μM, 100 μM and 250 μM of Ac4ManNAz followed by incubation with fluorescent cyclooctyne DBCO-PEG4-FAM, and (iii) effective electro-bioconjugation experiments NMeLum-N₃ (0.1 mM final conc.) followed by incubation with fluorescent cyclooctyne DBCO-PEG4-FAM (30′ (ON)+SPAAC).

FIG. 11 shows fluorescent positive cells (n=1), viability (n=1) (FIG. 11A) and mean fluorescence intensity measured by flow cytometry (n=1) (FIG. 11B) for (i) unmodified HeLa cells, (ii) unmodified HeLa cells incubated with DBCO-CD62L nanobody (0.01 mM final conc.) followed by incubation with labelled fluorescein anti-CD62L nanobody antibody (dilution 1/2500, HeLa+SPAAC+anti nanobody antibody), (iii) unmodified HeLa cells incubated with labelled fluorescein anti-CD62L nanobody antibody (dilution 1/2500, HeLa+anti nanobody antibody), and (iv) effective electro-bioconjugation experiments with NMeLum-N₃ (0.1 mM final conc.) followed by incubation with DBCO-CD62L nanobody (0.01 mM final conc.) and further incubation with labelled fluorescein anti-CD62L nanobody antibody (30′ ON+SPAAC+anti nanobody antibody). The labelled fluorescein anti-CD62L nanobody antibody (dilution 1/2500) was used to detect CD62L nanobody, confirming the covalent SPAAC conjugation between NMeLum-N₃ and the DBCO-CD62L (30′ ON+SPAAC+anti nanobody antibody).

FIG. 12 shows viability (n=1) (FIG. 12A) and mean fluorescence intensity measured by flow cytometry (n=1) (FIG. 12B) for (i) unmodified HeLa cells (HeLa), (ii) HeLa cells subjected to effective electro-bioconjugation experiments with GalNAc derivative of N-methyl luminol (NMeLum-GalNAc, 1 mM final conc.) (5′ ON), and iii) unmodified HeLa cells incubated with FITC-labeled Soybean lectin (20 μg/mL) (HeLa+Soybean FITC). FITC-labeled Soybean lectin was used to detect GalNAc on the surface of HeLa cells (FIG. 12B), confirming the covalent conjugation of NMeLum-GalNAc on the cell membrane (5′ ON).

### DETAILED DESCRIPTION FOR THE INVENTION

The Inventors conceived a new strategy enabling chemical modification of cell surface in both bacteria and eukaryotic cells such as mammalian cells. This method is based on the use of chemical reagents, namely N-alkyl luminol derivatives which are able to specifically react with tyrosine residues present in cell surface protein under specific conditions of activation.

First, the Inventors surprisingly showed that subjecting bacteria or eukaryotic cells to an electrochemical potential during one hour did not alter their viability.

Then, the Inventors identified NMeLum (N-methyl luminol) as being a coupling moiety of interest in the context of cell surface remodeling. NMeLum-containing ligands can be electro-activated at low potential regardless the substituent present on NMeLum aromatic ring. Of note, the potential used to oxidize NMeLum is low enough to avoid side reactions with most chemical groups other than phenol residue, which means that a large variety of ligands can be coupled through NMeLum moiety on cell surface. Besides, without to be bound by any theory, the Inventors are of the opinion that electro-activation of NMeLum moiety at low potential enables the formation of a stable nitrogen-centered radical which can specifically react through radical coupling with tyrosine residues present in surface proteins while avoiding the formation of highly reactive side-products and side-reactions with other amino acid residues (e.g. lysine, arginine) or other cell components (such as glycans or phospholipids) that may be present in cell surface.

Of note, the method of the Invention was shown to provide an effective surface modification in Gram-negative bacteria, Gram-positive bacteria and mammalian cells even if their surfaces are different from one another. Of note, the cells remain viable after the electrochemical coupling with the N-alkyl luminol reagent.

To the knowledge of the Inventors, electrochemical bioconjugation has been mostly used to chemically modify isolated proteins (see for instance Depienne et al., Chem. Sci., 2021, 12, 15374-15381) but not for modifying proteins present in complex environment such as cell surface in bacteria and mammalian cells.

As a proof of concept, the Inventors firstly assessed the ability of N-methyl luminol derivative to react with cell surface of living bacteria. Bacteria display an inner phospholipidic cell membrane surrounded by different types of outer cell wall architectures, i.e. a thick layer of peptidoglycans and teichoic acid for gram-positive (g+) bacteria, and a thin layer of peptidoglycans sandwiched by an exterior asymmetric bilayer of lipopolysaccharides and phospholipids for gram-negative bacteria (g−). Living E. coli (g−) and S. epidermidis (g+) were used as models.

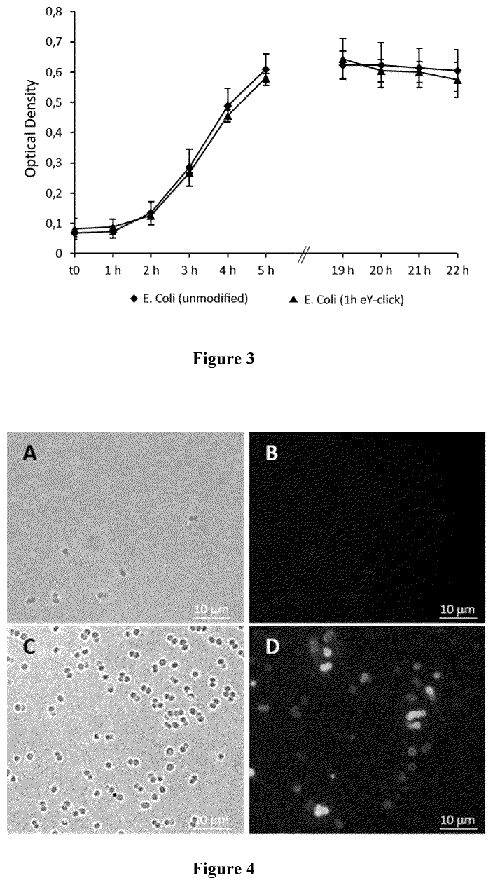

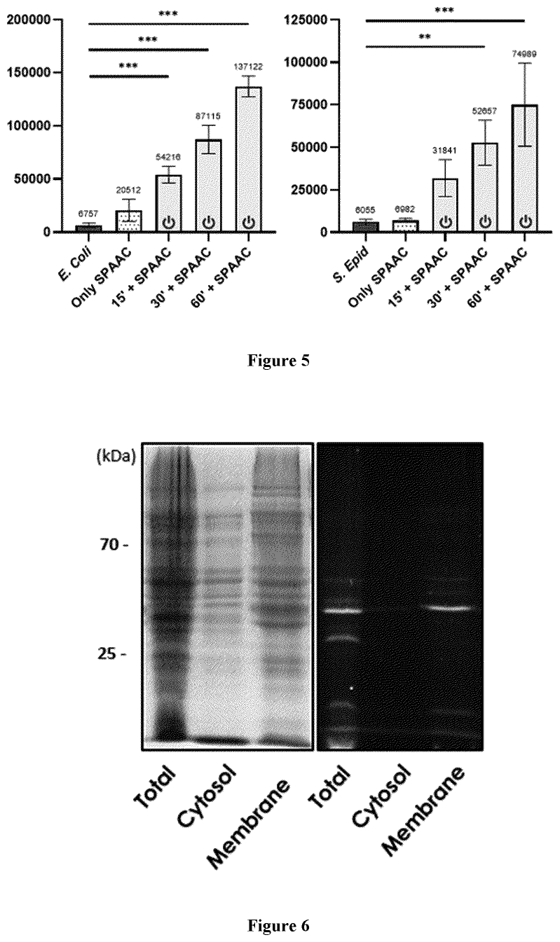

The bacteria were incubated with a N-methyl luminol derivative bearing an azido group (NMeLum-N₃) in phosphate buffer (pH 7.4) and under a low potential sufficient to oxidize the N-NMeLum-N₃ into a possible radical (e.g. 750 mV vs Ag/AgCl as reference electrode) during 15 to 60 minutes. The Inventors evidenced an effective time-dependent coupling of the N-methyl luminol moiety by fluorescence detection by using a strain-promoted azide-alkyne cyclization (SPAAC) with a constrained alkyne functionalized by a fluorescent probe (Fluorescein). Both flow cytometry and fluorescent microscopy showed a strong labeling of both bacteria species (FIG. 4 and FIG. 5). SDS-PAGE separation on gel of the cytosolic and membranes proteins after bacterial lysis and fractionation showed that only the membrane proteins were modified (FIG. 6). The Inventors further showed that the bacteria remain viable, and conserved their full capacity to replicate, even after electrochemical coupling (FIG. 3, Example 3).

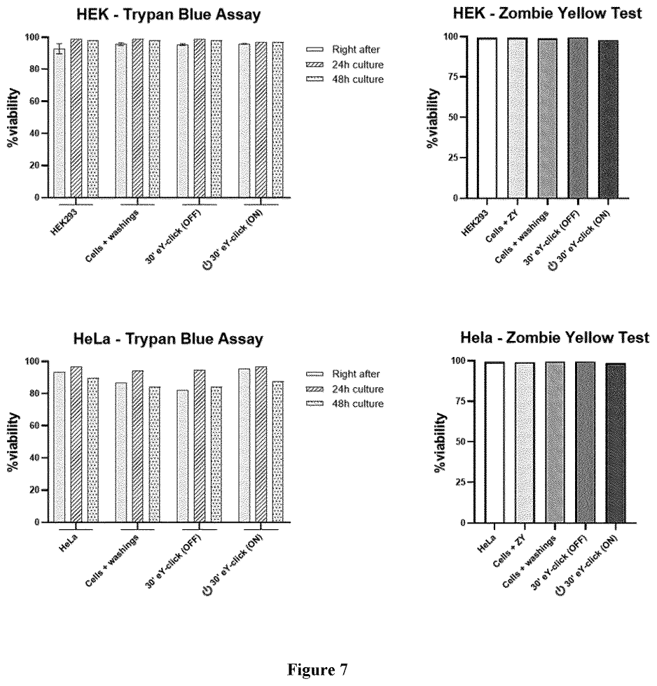

Then, the Inventors assessed the same strategy on mammalian cells, using HEK and HeLa cell lines as model. As shown in FIG. 7 and Example 8, the electrochemical coupling with NMeLum-N₃ followed by SPAAC with a strained alkyne bearing a fluorescent probe resulted in an effective labelling of the cell membrane as evidenced by flow cytometry and fluorescent microscopy. Of note, no significant labelling was detected intracellularly as evidenced by intra and extracellular staining with DAPI and label wheat germ agglutinin (WGA-647) respectively, as the labelling with NMeLum-N₃ was superimposable with that of WGA-647 but not with that of DAPI. Of note, the electrochemical coupling did not affect the cell viability since the cells conserved their ability to divide in cell culture conditions (FIG. 6). The Inventors also showed an effective electrochemical coupling in both cell lines with N-methyl luminol functionalized with biotin (NMeLum-biotin) as evidenced by complexation with streptavidin-FITC (Example 4).

Of note, no coupling was observed when the cells were merely incubated with N-methyl luminol derivatives without any electro-oxidative activation (Example 4).

The Inventors further showed that the method of the invention can be used for surface remodelling of adherent cells and cells growing in suspension: the method of the Invention enabling an effective surface bioconjugation of HELA, HEK, dendritic cells (DC 2.4), Jurkat and Expi-f without impairing cell viability (see Table 1 of the Example section and FIGS. 10, 11 and 12). Various functional moieties were immobilized on the cell surface, e.g. nanobodies (also called VHH), fluorescent labels, biotin and carbohydrates. Of note, the method of the Invention was shown to provide a higher cell surface modification level than standard glycol-engineering method (as evidenced by fluorescence activity) without impairing cell viability (FIG. 10).

To sum-up, the Inventors demonstrated that N-substituted luminol derivatives can be used to modify the cell surface of both bacteria and mammalian cells with specificity and efficacy and without impairing cell viability This method enables to decorate the cell surface with a wide variety of ligands such as saccharide and oligosaccharide moieties, biotin, fluorescent labels and proteins (e.g. nanobodies).

The chemical reagents are preferably used under electrochemical activation. Indeed, the Inventors developed an electrochemical bioconjugation process displaying many advantages such as a high reaction kinetics and conversion yield, a high chemo-selectivity towards tyrosine residues, no generation of by-products and implementation in biocompatible conditions. Besides, the electrochemical bioconjugation of the invention avoids using multiple chemical entities (such as oxidants, catalysts and/or scavengers), and at the end of the reaction, unreacted ligands can be easily removed by standard methods (such as dialysis). In addition, electrodes used in the electrochemical bioconjugation do not produce waste, can be reused several times, and allows to easily implement the process from a laboratory to industrial process scale. At last, contrary to the genetic and metabolic approaches described in the prior art, the method of the invention does not need any genetic manipulation of the cells or any pre-culture to perform the cell surface modification.

Without to be bound by any theory, the Inventors are of the opinion that the results shown for N-methyl luminol derivatives can be extrapolated to phenyl urazole (PhUr) and derivatives thereof including N-substituted PhUr derivatives such as N-methyl phenyl urazole, even if lower coupling yield is expected with N-substituted PhUr derivatives and even if lower specificity is expected with PhUr due to its electro-activation into less stable intermediate PTAD.

Accordingly, the Invention relates to a method for chemically modifying the surface of a cellular entity with a chemical reagent bearing a N-substituted luminol moiety, or a N-substituted PhUr moiety.

As mentioned further below, such a chemical reagent can comprise a functional moiety “M” linked directly or via a spacer Y to the N-substituted luminol moiety, or a N-substituted PhUr moiety.

The functional moiety can be linked to the aromatic ring of the N-substituted luminol moiety, or a N-substituted PhUr moiety.

In other embodiments, the functional moiety can be the substituent present on the “N” atom of the N-substituted luminol moiety, or a N-substituted PhUr moiety.

More specifically, the Invention relates to a method for chemically-modifying the surface of a cellular entity such as a cell, more precisely for chemically modifying at least one tyrosine residue present in a surface protein, said method comprising incubating the cell with a chemical reagent bearing a N-substituted luminol moiety, a N-substituted PhUr moiety under conditions conducive for coupling said chemical agent on the cellular entity surface, typically by reaction with a tyrosine residue present in a surface protein in the cell.

The cellular entity is preferably selected from a cell or an extracellular vesicle. More preferably the cellular entity is a cell.

As fully explained below, the cell can be of any type, in particular a bacteria or a mammalian cell.

The Invention also relates to a cellular entity (e.g. a cell) having its surface modified with a N-substituted luminol derivative or a N-substituted PhUr derivative (including phenyl urazole (PhUr) derivative) and its use in fields such as research, diagnostic and therapy.

In the context of the invention, the preferred moieties and derivatives are those of N-substituted luminol, such as N-alkyl luminol or N-benzyl luminol, because these derivatives combine a high selectivity and a high reactivity towards phenol groups, even if effective coupling can be obtained with PhUr derivatives.

Indeed, N-substituted luminol derivatives show a higher selectivity than PhUr, (because PhUr is electro-activated in the form of PTAD which less stable than the electro-activated form of N-substituted luminol and thus can lead to side reactions). On the other hand, N-substituted luminol derivatives show a similar selectivity than N-substituted PhUr but display a higher reactivity, leading to more effective coupling reactions.

### General Definitions

As used herein, the expression “about X” with X being a physical value (such as a voltage) corresponds to X±5%.

The term “Cₓ-C_{y}” in which x and y are integers, as used in the present disclosure, means that the corresponding hydrocarbon chain comprises from x to y carbon atoms. If, for example, the term C₁-C₆ is used, it means that the corresponding hydrocarbon chain may comprise from 1 to 6 carbon atoms, especially 1, 2, 3, 4, 5 or 6 carbon atoms. If, for example, the term C₂-C₅ is used, it means that the corresponding hydrocarbon chain may comprise from 2 to 5 carbon atoms, especially 2, 3, 4, or 5 carbon atoms.

As used herein, the term “alkyl” refers to a saturated, linear or branched aliphatic group. A preferred alkyl is a “C₁-C₆ alkyl”, which refers to an alkyl having 1 to 6 carbon atoms. Examples of alkyl (or C₁-C₆ alkyl) include, but are not limited to methyl, ethyl, propyl, isopropyl, butyl, isobutyl, pentyl, hexyl.

As used herein, the term “alkene” or “alkenyl” refers to an unsaturated, linear or branched aliphatic group, having at least one carbon-carbon double bond. A preferred alkene is a “C₂-C₆ alkene”, which refers to an alkene having 2 to 6 carbon atoms. Examples of alkene (or C₂-C₆ alkene) include for instance ethenyl, propenyl, butenyl, pentenyl, or hexenyl, preferably ethenyl (—CH═CH₂).

As used herein, the term “alkyne” or “alkynyl” refers to an unsaturated, linear or branched aliphatic group, having at least one carbon-carbon triple bond. A preferred alkyne is “C₂-C₆ alkyne”, which refers to an alkyne having 2 to 6 carbon atoms. Examples of alkyne (or C₂-C₆ alkyne) include for instance ethynyl, propynyl, butynyl, pentynyl, or hexynyl, preferably ethynyl (—C≡CH).

As used herein, the term “alkoxy” refers to an alkyl as defined herein, attached to the remainder of the molecule via an ether bond (—O—). In other words, an alkoxy can be written “—O-alkyl”. A preferred alkoxy is a C₁-C₆ alkoxy, which has 1 to 6 carbon atoms. Examples of alkoxy (or C₁-C₆ alkoxy) include for instance, methoxy, ethoxy, propoxy, isopropoxy, butoxy, pentoxy, hexyloxy.

As used herein, the term “alkylthio” refers to an alkyl as defined herein, attached to the remainder of the molecule via a thioether bond (—S—). In other words, an alkylthio can be written “—S-alkyl”. A preferred alkylthio is a C₁-C₆ alkylthio, which has 1 to 6 carbon atoms. Examples of alkylthio (or C₁-C₆ alkylthio) include for instance, methylthio, ethylthio, propylthio, isopropylthio, butylthio, pentylthio, hexylthio.

As used herein, the term “alkylamino” refers to an alkyl as defined herein, attached to the remainder of the molecule via an amino bond (—NH—). In other words, an alkylamino can be written “—NH-alkyl”. A preferred alkylamino is a C₁-C₆ alkylamino, which has 1 to 6 carbon atoms. Examples of alkylamino (or C₁-C₆ alkylamino) include for instance, methylamino, ethylamino, propylamino, isopropylamino, butylamino, pentylamino, hexylamino.

As used herein, the term “carbocycle” (or “carbocyclic group”) refers to a saturated or unsaturated, aliphatic or aromatic, mono-, bi- or tri-cyclic hydrocarbon group. The carbocyclic group may be in particular a cycloalkyl, a cycloalkenyl, or an aryl.

As used herein, the term “cycloalkyl” refers to a saturated mono-, bi- or tri-cyclic aliphatic group. It also includes fused, bridged, or spiro-connected cycloalkyl groups. The term “C₃-C₆ cycloalkyl” refers to a cycloalkyl having 3 to 6 carbon atoms. Examples of cycloalkyl (or C₃-C₆ cycloalkyl) include, but are not limited to cyclopropyl, cyclobutyl, cyclopentyl, cyclohexyl. The term “cycloalkyl” may also refer to a bridged carbocyclyl such as bicyclo[2,2,1]heptanyl, bicyclo[2,2,2]octanyl, or adamantyl.

As used herein, the term “cycloalkenyl” refers to an unsaturated mono-, bi- or tri-cyclic aliphatic group, comprising at least one carbon-carbon double bond. It also includes fused, bridged, or spiro-connected cycloalkenyl groups. The term “C₃-C₆ cycloalkenyl” refers to a cycloalkenyl having 3 to 6 carbon atoms. Examples of cycloalkenyl (or C₃-C₆ cycloalkenyl) include, but are not limited to cyclopentenyl, and cyclohexenyl.

As used herein, the term “heterocycle” corresponds to a saturated or unsaturated, aliphatic or aromatic, mono-, or polycyclic (e.g. bi-, tri-, or tetra-cyclic) group, comprising at least one heteroatom such as nitrogen, oxygen, or sulphur atom. In the case of a bi- or tricycle, wherein the cycles can be fused, bridged or have a spiro configuration. Advantageously, the heterocycle comprises between 3 and 20 ring atoms, for instance between 3 and 6 ring atoms, wherein at least one of the ring atoms is a heteroatom such as nitrogen, oxygen or sulphur atom. In some embodiments, the “heterocycle” is a heterocycloalkyl, a heterocycloalkenyl, or a heteroaryl. In some embodiments, the heterocycle is a heterocycloalkyl, a heterocycloalkenyl, or a heteroaryl, fused with one or more carbocyclic or heterocyclic moieties (for instance, a heteroaryl fused with a cycloalkyl).

As used herein, the term “heterocycloalkyl” corresponds to a cycloalkyl group as above defined in which at least one carbon atom has been replaced with a heteroatom such as nitrogen, oxygen, or sulphur atom.

As used herein, the term “heterocycloalkenyl” corresponds to a cycloalkenyl group as above defined in which at least one carbon atom has been replaced with a heteroatom such as nitrogen, oxygen, or sulphur atom.

Examples of heterocycles, which are heterocycloalkyl or heterocycloalkenyl, include, but are not limited to, aziridinyl, azepanyl, diazepanyl, dioxolanyl, benzo [1,3]dioxolyl, azetidinyl, oxetanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, pyrimidinyl, oxozolinyl, oxazolidinyl, isoxazolinyl, isoxazolidinyl, thiooxetanyl, thiopyranyl, thiomorpholinyl, thiazolinyl, thiazolidinyl, isothiazolinyl, isothiazolidinyl, dihydropyranyl, dihydrofuranyl, dihydrothiopyranyl, dihydrothiophenyl, dihydropiperidinyl, tetrahydropiperidinyl, tetrahydrothiopyranyl, tetrahydropyranyl, tetrahydrofuranyl, and tetrahydrothiophenyl.

As used herein, the term “aryl” refers to an aromatic ring system, which preferably has 6-14 atoms, having at least one ring having a conjugated pi electron system and which optionally may be substituted. An “aryl” may contain more than one aromatic ring such as fused ring systems or an aryl group substituted with another aryl group. Aryl encompass, without being limited to, phenyl, anthracenyl, naphthyl, indenyl, divalent biphenyl.

“Heteroaryl” refers to a heteroaryl group. “Heteroaryl” refers to a chemical group, preferably having 5-14 ring atoms, wherein 1 to 4 heteroatoms are ring atoms in the aromatic ring and the remainder of the ring atoms being carbon atoms. Suitable heteroatoms include oxygen, sulfur, nitrogen, phosphorus, and selenium. Examples of heterocycles, which are heteroaryl groups, include triazolyl, furanyl, thienyl, pyridyl, pyrrolyl, N-alkyl pyrrolyl, pyridyl-N-oxide, pyrimidyl, pyrazinyl, imidazolyl, benzimidazolyl, benzofuranyl, benzothiophenyl, quinazolinyl, and quinolinyl.

Examples of bicyclic heteroaryl groups encompass, without being limited to bicyclic heteroaryl groups that may be mentioned include 1H-indazolyl, benzo[1,2,3]thiadiazolyl, benzo[1,2,5]thiadiazolyl, benzothiophenyl, imidazo[1,2-a]pyridyl, quinolinyl, indolyl and isoquinolinyl groups.

As used herein, a (C₆-C₁₄ aryl)-(C₁-C₃ alkyl) refers to a C₁-C₃ alkyl as defined herein, substituted by at least one (preferably, one only) C₆-C₁₄ aryl as defined herein. A preferred (C₆-C₁₄ aryl)-(C₁-C₃ alkyl) is phenylmethyl (namely benzyl).

As used herein, the term “alkanoyl” refers to an alkyl as defined herein, attached to the remainder of the molecule via an oxo group (—C(O)—). In other words, an alkanoyl can be written “—C(O)-alkyl”. A preferred alkanoyl is a C₁-C₆ alkanoyl, which has an alkyl chain of 1 to 6 carbon atoms. Examples of alkanoyl (or C₁-C₆ alkanoyl) include for instance, methanoyl, ethanoyl, propanoyl, isopropanoyl, butanoyl, pentanoyl, hexanoyl.

As used herein, the term “acylamino” refers to a group of formula R—C(O)—NH— wherein R is a hydrocarbon group such as C₁-C₆ alkyl, a C₃-C₁₂ cycloalkyl or an aryl. A preferred acylamino is a C₁-C₆ acyl amino, which has a hydrocarbon chain of 1 to 6 carbon atoms.

As used herein, the term “ester” or “carboxy ester” refers to a —C(O)OR′ or R′C(O)O— group, wherein R′ is any hydrocarbon group, such as a C₁-C₆ alkyl, a C₃-C₁₂ cycloalkyl or an aryl. A preferred ester is a C₁-C₆ ester, which has a hydrocarbon chain of 1 to 6 carbon atoms.

As used herein, an “alkoxycarbonyloxy” refers to a R″—C(O)—O— group where R″ is an alkoxy.

As used herein, the term “halogen” includes chlorine, fluorine, iodine, bromine, preferably chlorine or fluorine.

As used herein, the term “aminoalkyl” refers to an alkyl as defined above, substituted by one or more (preferably one) amino (—NH₂) group.

As used herein, the term “alkylaminoalkyl” refers to an alkyl as defined above, substituted by one or more (preferably one) alkylamino group as defined above.

As used herein, the term “hydroxyalkyl” refers to an alkyl as defined above, substituted by one or more (preferably one) hydroxy (—OH) group.

As used herein, the term “alkoxyalkyl” refers to an alkyl as defined above, substituted by one or more alkoxy as defined above.

As used herein, the term “thioalkyl” refers to an alkyl as defined above, substituted by one or more (preferably one) —SH group.

As used herein, the term “haloalkyl” refers to an alkyl as defined above, substituted by one or more halogen atoms.

“Substituted” or “optionally substituted” includes groups substituted by one or several substituents, typically 1, 2, 3, 4, 5 or 6 substituents. For instance, the substituents may be independently selected from C₁-C₆ alkyl, aryl, C₃-C₆ cycloalkyl, C₃-C₆ cycloalkenyl, C₂-C₆ heterocycle, C₁-C₆ alkoxy, C₁-C₆ alkylamino, C₁-C₆ aminoalkyl-, C₁-C₆ alkylaminoalkyl-, —N₃, —NH₂, —F, —I, —Br, —Cl, —CN, C₁-C₆ alkanoyl, C₁-C₆ carboxy esters, C₁-C₆ acylamino, —COOH, —CONH₂, OH, —NO₂, —SO₃H, C₁-C₆ hydroxyalkyl, C₁-C₆ haloalkyl, C₁-C₆ alkylthio, C₁-C₆ thioalkyl, C₂-C₁₀ alkoxyalkyl, C₂-C₆ alkoxycarbonyloxy, —CN, —CF₃ and C₂-C₆ alkoxyalkyl. Preferred substituents are halogens, —NO₂, C₁-C₃ alkyl, C₁-C₃ alkoxy, C₁-C₃ hydroxyalkyl, and C₁-C₃ haloalkyl.

As used herein, N-substituted luminol refers to the compound of the following formula:

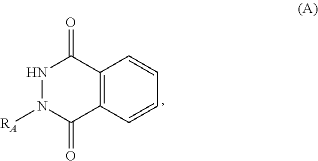

wherein R_{A} is a substituent which can be of any type.

As used herein, a N—(C₁-C₆ alkyl) luminol refers to a compound of formula (A) wherein R_{A} is C₁-C₆ alkyl. As used herein, a N-methyl luminol refers to a compound of formula (A) wherein R_{A} is a methyl.

As used herein, a N—(C₆-C₁₄ aryl optionally substituted) luminol refers to a compound of formula (A) wherein R_{A} is a C₆-C₁₄ aryl optionally substituted.

As used herein, a N—[(C₆-C₁₄ aryl)-(C₁-C₃ alkyl) optionally substituted] luminol refers to a compound of formula (A) wherein R_{A} is (C₆-C₁₄ aryl)-(C₁-C₃ alkyl) optionally substituted.

As used herein, a N-benzyl luminol refers to a compound of formula (A) wherein R_{A} is a benzyl.

N-methyl luminol refers to the compound wherein R_{A} is a methyl. N-methyl luminol can also be called “2,3-Dihydro-2-methyl-1,4-phthalazinedione” and has the following CAS number: 18393-54-9.

As used herein, “N-substituted luminol derivative” refers to N-substituted luminol as defined above, having optionally one or several additional substituents on the benzene ring of the phthalazinedione group.

As used herein, N-substituted phenylurazole (PhUr) refers to the compound of formula (P):

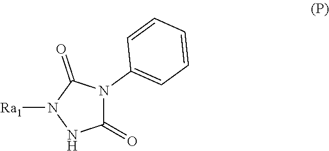

wherein Ra₁ is a substituent of any type

As used herein, “N-substituted PhUr derivative” refers to N-substituted PhUr as defined above, having optionally one or several substituents on the phenyl group.

The phrase “optionally substituted” can be replaced with the phrase “substituted or unsubstituted” throughout this application.

As used herein, “a cellular entity” typically refers to any entity having an external envelop similar to a cell wall, a cell membrane or a cell organelle. A cellular entity encompasses, without being limited to, cells and extracellular vesicles.

As used herein, “a cellular entity” typically refers to any entity having an external envelop similar to a cell wall, a cell membrane or a cell organelle.

A cellular entity encompasses, without being limited to, cells and extracellular vesicles.

As used herein, the term “cell” refers to both prokaryotic cells such as bacteria and archaea and eukaryotic cells including animal cells, plant cells, yeast, fungi, and algae. The cell may be of any type. Preferably the cells are either bacteria or mammalian cells.

The surfaces of bacterial and mammalian cells are different from one another. The cell envelope of Gram-negative bacteria consists of an outer membrane as the outermost layer, followed by a peptidoglycan cell wall and an inner membrane. The outer membrane is a defining feature of Gram-negative bacteria, which is a lipid bilayer. Phospholipids are found only on the inner leaflet, while the outer leaflet is principally composed of lipopolysaccharides (LPS), lipoproteins, and b-barrel proteins. Gram-positive bacteria lack an outer membrane, but have a much thicker peptidoglycan cell wall as the outermost surface. Some proteins on the surface of Gram-positive bacteria are covalently attached to or are associated with peptidoglycan or teichoic acids. Others span the cell membrane or contain a lipid tail for anchoring to the membrane.

The surface structure of mammalian cells, the plasma membrane, consists of the fundamental phospholipid bilayer with protein molecules embedded within. Many membrane proteins and lipids are conjugated to polysaccharides, which comprise the cellular coat of all cells. As used herein, “cell surface” in eukaryotic cells, in particular in mammalian cells refer to the plasma membrane, preferably to its extracellular face. “Cell surface” in bacteria refer to the cell membrane in Gram-positive bacteria, preferably to the extracellular face of the cell membrane, and to the inner membrane, the periplasm, the outer membrane, preferably the extracellular face of the outer membrane.

As used herein, the term “extracellular vesicle” (EV) refers to phospholipid bilayer-delimited particles but, which cannot replicate unlike cells. The phospholipid lipid layer generally includes membrane or transmembrane proteins. Preferably, the Evs are delimited by a phospholipid bilayer membrane. The term “Extracellular vesicle” encompasses vesicles released from a cell. An extracellular vesicle can be spontaneously released by a cell or artificially secreted. In some embodiments, the phospholipid layer delimited the EV derives from the cell membrane of its parent cell. An EV can carry a cargo of proteins, nucleic acids such as mRNA or miRNA, lipids, metabolites, and even organelles from the parent cell. The parent cell can be of any type, including bacterial, fungal, plant and animal cells. In particular embodiments, the extracellular vesicle can be from a mammal cell, including from a human cell. In some embodiments, the EV derives from megakaryocytes, blood platelets, monocytes, neutrophils, tumor cells, macrophages, and placenta cells. In multicellular organisms, EV can be found in tissues (e.g in the interstitial space) and in body fluids. EV can be also produced and isolated in vitro from cell culture, in particular from stem cell culture.

Extracellular vesicles may have a diameter of at most 1 000 nm, preferably of at most 500 nm e.g. from 20 to 300 nm. Extracellular vesicles encompass, without being limited to, exosomes, microvesicles (also called ectosomes or microparticles) and apoptotic bodies.

In particular, the extracellular vesicle may be a cell membrane vesicle, e.g. such as exosomes released by cells, in particular by mammal cells such as human cells.

As used herein, a “surface component” refers to any constituent present in the “cellular entity surface”, preferably the “cell surface” which include peptidoglycans, polysaccharides, lipids and the like. The surface component modified by the method of the invention is preferably a protein, such as a membrane-bound protein, a glycoprotein, a transmembrane protein (e.g. beta-barrel proteins in Gram-negative bacteria), a lipoprotein and the like. In Gram-positive bacteria, surface proteins can be also present as proteins covalently linked or associated with peptidoglycans or teichoic acids.

As used herein, a “surface tyrosine residue” refers to a tyrosine residue present in a surface component, preferably in a surface protein which is accessible for covalent coupling.

### 1. Method for Chemically Modifying the Surface of a Cellular Entity According to the Invention

In a first aspect, the Invention relates to a method for chemically modifying the surface of a cellular entity (preferably a cell) with a chemical reagent bearing a N-substituted luminol moiety or a N-substituted PhUr moiety.

As mentioned further below, such a chemical reagent can comprise a functional moiety “M” linked directly or via a spacer Y to the luminol moiety or to the PhUr derivative.

More specifically, the Invention relates to a method for chemically-modifying the surface of cellular entity (preferably a cell), more precisely for chemically modifying at least one tyrosine residue present in a surface protein of the cellular entity (preferably the cell), which comprises incubating said cellular entity (preferably the cell) with a chemical reagent bearing a N-substituted luminol moiety or a N-substituted PhUr moiety under conditions conducive for coupling said chemical agent to the surface of the cellular entity (preferably the cell), typically by reaction with a tyrosine residue present in a surface protein in the cell.

The possible N-substituted luminol moiety present in the chemical reagent is typically selected from the group consisting of N—(C₁-C₆ alkyl) luminol, N—(C₆-C₁₄ aryl optionally substituted) luminol or N—[(C₆-C₁₄ aryl)-(C₁-C₃ alkyl) optionally substituted] luminol, preferably a N-methyl or N-benzyl luminol moiety, more preferably N-methyl luminol moiety.

The possible N-substituted PhUr derivative present in the chemical reagent is typically selected from the group consisting of N—(C₁-C₆ alkyl) PhUr, N—(C₆-C₁₄ aryl optionally substituted) PhUr or N—[(C₆-C₁₄ aryl)-(C₁-C₃ alkyl) optionally substituted] PhUr, preferably N—(C₁-C₆ alkyl) PhUr or PhUr.

In these embodiments, the functional moiety “M”, when present, is linked directly or via a spacer Y on the aromatic ring of said N-substituted PhUr or luminol derivatives.

In other embodiments, the functional moiety “M”, when present, is linked directly or via a spacer Y on a “N” atom of luminol or PhUr moieties so as to give a N-substituted derivative which optionally have additional substituent(s) on the aromatic ring.

In a particular embodiment, the chemical reagent bears a N-substituted luminol and can be of formula (I):

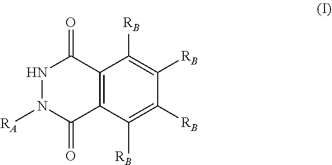

Wherein R_{A} and R_{B} are defined further below.

Preferred chemical reagents of formula (I) are those of formula (Ia):

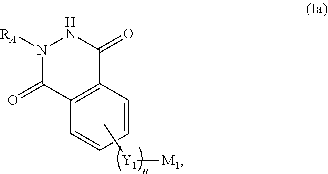

Wherein Y₁ and M₁ are as defined further below and n is 0 or 1.

In a particular embodiment, said chemical reagent bears a N-substituted PhUr and can be of formula (IP):

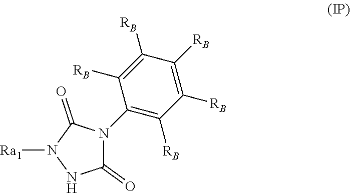

Wherein Ra₁ and R_{B} are defined further below.

Preferred chemical reagents of formula (IP) are those of formula (IPa):

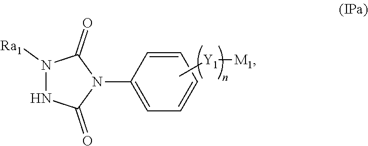

Wherein Y₁ and M₁ are as defined further below and n is 0 or 1.

### a) Conditions to Implement the Method of the Invention

The coupling conditions used in the method of the Invention enable the in-situ activation of N-substituted luminol moiety or the N-substituted PhUr moiety into an oxidized entity able to react with cellular entity surface i.e. a cellular entity surface component such as a surface protein, and in particular with phenol group present in tyrosine residues in surface proteins.

In the case of N-substituted luminol derivative, the coupling conditions preferably enable the formation of nitrogen-centered radical in the N-substituted luminol moiety. Such an oxidation can be performed by any methods known by the skilled artisan to activate N-substituted luminol preferably into single radical, for instance by an enzymatic system such as horse radish peroxidase/H₂O₂ or laccase/O₂ or by electrochemistry.

Similarly, activation of N-substituted PhUr can be performed by an enzymatic system such as horse radish peroxidase/H₂O₂ or laccase/O₂ or by electrochemistry. When Ra₁ is H, activation with a chemical oxidant such as NBS and DBDMH can be also contemplated.

The Inventors showed that chemical reagents bearing N-substituted luminol have an oxidation potential of about 0.6 V versus Saturated Calomel Electrode (SCE) regardless the substituent present on the nitrogen atom or on the aromatic ring of the N-substituted luminol and that the resulting oxidized entity formed at said potential is reactive towards the phenyl group of tyrosine. Besides, the Inventors showed that subjecting cells to such a low voltage does not impair their viability.

At last, the Inventors managed to specifically and efficiently modify cell surface of both bacterial and mammalian cells by incubating the cells with a chemical reagent bearing a N-substituted luminol moiety under a constant low potential difference. Accordingly, the method of the Invention is carried out by electrochemistry, i.e. the chemical reagent bearing the N-substituted luminol is activated by the application of a low voltage.

As used herein, “electrochemistry” refers to branch of chemistry wherein the reaction between entities of interest is triggered by subjecting said entities to an electrical potential difference.

In a particular embodiment, the method of the Invention refers to a method for chemically-modifying the surface of a cellular entity (preferably a cell) with a chemical reagent bearing a N-substituted luminol moiety or a N-substituted PhUr moiety by electrochemistry, namely by contacting the cellular entity (preferably the cell) and the chemical reagent under a potential difference enabling the electro-activation of the chemical reagent. Typically, the potential difference is such that it does not significantly impair the viability of the cell.

As used herein, the “electro-activation of the chemical reagent of the invention” refers to the oxidation of chemical reagent of the invention into an oxidized form able to react with a cell surface component, such as a surface protein and more precisely the phenyl group present in a tyrosine residue from a surface protein, by means of a potential difference.

In a particular embodiment, the chemical reagent of formula (I) is incubated with the cellular entity (preferably the cell) so as to obtain at least one chemically-modified tyrosine residue in of formula (B) in the surface of the cellular entity (preferably the cell):

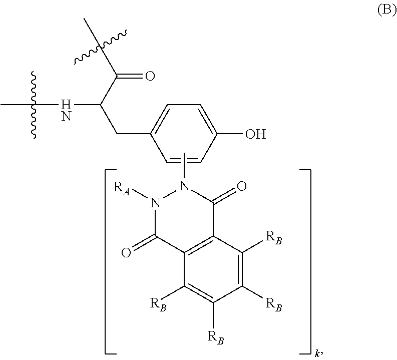

Wherein k being 1 or 2 and R_{A} and R_{B} being as defined further below.

In another embodiment, the chemical reagent of formula (IP) is incubated with the cellular entity (preferably the cell) so as to obtain at least one chemically-modified tyrosine residue in of formula (BP) in the cell surface:

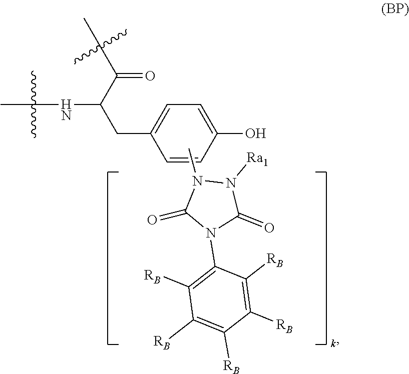

Wherein:
k being 1 or 2,
Ra₁ is H or R_{A}, preferably H or a C₁-C₆ alkyl
and R_{B} and R_{A} are as defined further below.

The electrochemical conditions (also called herein the potential conditions) are selected so as to enable the oxidation of the chemical reagent of formula (I) or (IP) without impairing the integrity of the cellular entity. In some embodiments, the electrochemical conditions are selected so as to enable the oxidation of the chemical reagent of formula (I) or (IP) without significantly impairing the cell viability.

As used herein, the “potential conditions” refer to a potential difference with respect to a reference electrode which enables to oxidize the chemical reagent preferably into a radical.

This potential difference is close to the oxidation potential of the chemical reagent (determined with respect to the reference electrode). Typically the potential difference to apply is generally included in a range from the oxidation potential of the chemical reagent (OP) minus 200 mV to OP plus 500 mV, preferably from OP minus 150 mV to OP plus 400 mV.

In a particular embodiment, the potential difference to apply is equal or substantially close to the oxidation potential (OP) of the chemical reagent, e.g. equal to the oxidation potential of the chemical reagent±200 mV or ±150 mV, more preferably ±100 mV such as ±50 mV or ±25 mV.

The potential difference to apply to oxidize the chemical reagent in the method of the invention varies, among others, depending on the reference potential of the reference electrode used to implement the method.

For a given chemical reagent, the oxidation potential can be identified by standard proceeding well known by the skilled artisan, such as cyclic voltammetry. One can refer to the method described in the Example section (see Example 2).

Typically, the cell and the chemical reagent are subjected to a potential difference (or equivalently a “voltage”). Any suitable electrochemical device may be used to apply the voltage. In some embodiments, the voltage is applied by means of an electrochemical device comprising a three-electrode system, namely a working electrode, an auxiliary electrode and a reference electrode. The working electrode refers to the electrode on which the reaction of interest occurs. Depending on whether the reaction on the electrode is a reduction or an oxidation, the working electrode is called cathodic or anodic, respectively. In the context of the invention, the working electrode is the anode. In the context of the invention, the auxiliary electrode (also called counter electrode) is the cathode. The electrochemical device can further comprise a vial, electrical connector means and a mean to control the potential difference between the reference electrode and the working electrode, typically a potentiostat.

For instance, at the laboratory scale, the cells are resuspended in an appropriate buffer. The chemical reagent is added to the cells and the resulting mixture is placed in an appropriate vial (e.g. a plastic or glass vial) in which three electrodes are plunged. The three electrodes are connected with appropriate electrical connector means to a potentiostat. The auxiliary electrode may be isolated e.g. by using a glass frit in order to avoid the formation of by-products. The potentiostat is used to maintain the potential difference between the reference electrode and the working electrode at a constant value enabling the selective oxidation of the chemical reagent.

The anodes, cathodes and reference electrodes that can be used in electrochemical processes are well-known to the skilled artisan.

Examples of material from which anodes can be made include, but are not limited to, carbon (e.g. glassy carbon or graphite), lead bronze, tungsten, niobium, copper, magnesium, titanium, zinc, stainless steel, platinum, gold, silver, aluminium, boron doped diamond, tin, nickel, cobalt, preferably carbon anode (e.g. glassy carbon or graphite).

Examples of material from which cathodes can be made include, but are not limited to, nickel, platinum, silver, lead bronze, tungsten, niobium, copper, magnesium, titanium, zinc, stainless steel, gold, aluminium, boron doped diamond, tin, nickel, cobalt, preferably platinum cathode.

Examples of reference electrodes include, but are not limited to, Standard hydrogen electrode (SHE), Normal hydrogen electrode (NHE), Reversible hydrogen electrode (RHE), Saturated calomel electrode (SCE), Copper-copper(II) sulfate electrode (CSE), Silver chloride electrode, Palladium-hydrogen electrode, dynamic hydrogen electrode (DHE), and Mercury-mercurous sulfate electrode (MSE), preferably silver chloride electrode.

The potentials of reference electrodes are easily available in reference handbooks. Examples of reference potentials, defined with respect to the SHE, are herein provided (non-exhaustive list):
Standard hydrogen electrode (SHE): E=0.000 V
Normal hydrogen electrode (NHE): E≈0.000 V,
Reversible hydrogen electrode (RHE): E=0.000 V,
Saturated calomel electrode (SCE): E=+0.241 V saturated,
Copper-copper (II) sulfate electrode (CSE): E=+0.314 V,
Silver chloride electrode: E=+0.197 V in saturated KCl,
Silver chloride electrode: E=+0.210 V in 3.0 mol KCl/kg,
Silver chloride electrode: E=+0.22249 V in 3.0 mol KCl/L,

Preferably, the electrochemical device used to apply the voltage comprises:
a silver chloride electrode as a reference electrode,
a platinum electrode as a cathode, and
a carbon (e.g. graphite) electrode as an anode.

The shape and the morphology of the electrodes is not particularly limited. For instance, a carbon electrode can be reticulated, laminar or crucible. Examples of electrode shapes include, but are not limited to, a plate, a wire, or a flat rod. The size of the electrodes can be adjusted by the skilled artisan, depending on the scale of the process, in particular the volume of the incubation medium. The surface of the electrodes in contact with the incubation medium can also be adjusted by the skilled artisan. Preferably, at least 20% 30%, 40%, 50%, 60%, 70%, 80%, 90% of the total surface of each electrode is in contact with the incubation medium.

The voltage may be set and controlled by any suitable device, typically a potentiostat connected to the electrodes (i.e. cathode, anode, reference electrode) of the electrochemical device.

The voltage is set at a value that allows the activation (e.g. the oxidation) of the N-substituted luminol moiety of the chemical reagent. The voltage to be applied can be easily determined by the skilled artisan, in particular through the determination of the oxidation potential of the chemical reagent versus a reference electrode, in the incubation medium of interest (i.e herein the buffer of interest) by cyclovoltammetry.

As mentioned above, the potential difference to apply during the incubation is generally included in a range from the oxidation potential of the chemical reagent (OP) minus 200 mV to OP plus 500 mV, preferably from OP minus 150 mV to OP plus 400 mV.

In a particular embodiment, the potential difference to apply is equal or substantially close to the oxidation potential (OP) of the chemical reagent, e.g. equal to the oxidation potential of the chemical reagent±150 mV, more preferably ±100 mV such as ±50 mV or ±25 mV.

In some embodiments, the incubation is carried out under a voltage equal to such oxidation potential of the chemical reagent±150 mV, preferably ±100 mV or ±50 mV, more preferably 40 mV or ±30 mV, even more preferably ±20 mV or ±10 mV.

Generally, the oxidation potential of a chemical reagent of formula (I) is about 0.75 V vs. Ag/AgCl in saturated KCl. The incubation may be carried out under a voltage selected from values in the range from +0.55 V to 1.25 V vs. Ag/AgCl in saturated KCl, preferably between +0.60 V to 1.0 V vs. Ag/AgCl in saturated KCl, more preferably about +0.70 V to +0.85 V such as from 0.70 V to 0.80 V e.g. about 0.75 V vs. Ag/AgCl in saturated KCl, wherein the reference potential of Ag/AgCl in saturated KCl is +0.197 V.

As another example, the incubation may be carried out under a voltage between +0.55 and +0.65 V vs. SCE, preferably between +0.57 and +0.63 V vs. SCE, more preferably about +0.6 V vs. SCE, wherein the reference potential of SCE is +0.241 V.

As a further example, the oxidation potential of a PhUr derivative, i.e a chemical reagent of formula (IP) with Ra₁ is H, is generally about +0.45 V vs. Ag/AgCl in saturated KCl. The incubation may be carried out under a voltage selected from values in the range from +0.25 to +0.95 V vs. Ag/AgCl in saturated KCl, preferably from +0.30 to +0.75 V vs. Ag/AgCl in saturated KCl, more preferably from 0.40 V to 0.50 V such as about +0.45 V vs. Ag/AgCl in saturated KCl, wherein the reference potential of Ag/AgCl in saturated KCl is +0.197 V.

As another example, the oxidation potential of a PhUr derivative, i.e a chemical reagent of formula (IP) with Ra₁ is R_{A} is generally about +0.65 V vs. Ag/AgCl in saturated KCl. The incubation may be carried out under a voltage selected from +0.45 V to 1.15 V vs. Ag/AgCl in saturated KCl, preferably between +0.50 V to 0.90 V vs. Ag/AgCl in saturated KCl, more preferably about +0.60 V to +0.75 V such as from 0.60 V to 0.70 V e.g. about 0.65 V vs. Ag/AgCl in saturated KCl, wherein the reference potential of Ag/AgCl in saturated KCl is +0.197 V.

The incubation may be performed in an aqueous buffer having a pH from 5 to 11, preferably from 7 to 10, e.g. from 7.0 to 8.0, e.g. about 7.5. The concentration in buffer agent is at least 30 mM, preferably at least 50 mM and up to 1 M.

The buffer may be selected from appropriate biocompatible buffers, e.g TRIS buffer, sodium carbonate-sodium bicarbonate buffer, phosphate buffer e.g. PBS or Dulbecco's phosphate-buffered saline (dPBS), or Good's buffer.

The incubation time during which the potential difference is applied may vary depending on several parameters such as (i) the volume of the buffer and the dimension of the vial, (ii) the surface of the electrodes being in contact with the incubation medium (iii) the amounts of chemical reagent and the amount of cells to chemically modify, (iv) the chemical reagent, (v) the solubility of the chemical reagent, (vi) the voltage and (vii) the stirring rate.

The incubation may last from few seconds to several hours, for instance from 1 min to 240 min, for instance from 5 min to 180 min, e.g. from 10 min to 120 min or from 10 min to 90 min such as from 15 min to 30 min, from 30 min to 45 min, from 45 min to 60 min, from 60 min to 75 min, and from 75 min to 90 min.

The temperature of incubation is typically from 10° C. to 40° C. The temperature of incubation is fixed depending on the cell to modify. Generally, the incubation is performed in room temperature.

In some embodiments, the reaction is performed under stirring, preferably under orbital stirring. In some other embodiments, the reaction is performed without stirring.

The cell titre varies depending on the cells used. For instance, for mammal cell lines such as Hela or HEK, the cell titre may be from 0.5 á 1.5^{E}6 cells/mL.

The concentration of the chemical reagent may be from 0.01 mM to 50 mM, for instance from 0.1 mM and 20 mM, such as from 0.1 to 10.0 mM or such as 0.1 to 5.0 mM such as 1 mM or 2 mM.

The method of the invention is preferably implemented in vitro (e.g. on resuspended cells or isolated cells) or ex vivo (e.g. in the case of tissue sample).

### b) the Cellular Entity

As used herein, “a cellular entity” typically refers to any entity having an external envelop similar to a cell wall, a cell membrane or a cell organelle.

A cellular entity encompasses, without being limited to, cells and extracellular vesicles.

The method of the Invention can be implemented on any type of cells, including procaryotic or eucaryotic cells. The cells can be recombinant cells or naturally-occurring cells. When the cell is a pathogen for therapeutic application (e.g. to be used as drug carrier), the virulence of the pathogen can have been attenuated by any suitable means including by genetic mutations.

The cell may be of any type. The cell is selected depending on its intended use, e.g. for research purpose, imaging purpose or therapy purpose. The cell is typically an isolated cell or a cell present in a cell culture, in a sample, in an ex vivo tissue or organ or in an organoid.

As used herein, “cell culture” includes, without being limited to, a cell culture of a cell line, a primary culture, a mixed culture (or co-culture) comprising several cell types, and an organotypic culture.

In some embodiments, the cell is a bacterium.

Bacteria encompass both gram+ and gram− cells. The bacteria can be pathogenic bacteria for human (i.e responsible for infections in human) or pathogenic for other living beings. The bacteria can be naturally occurring in the environment. As another example, bacteria can belong to human microbiota.

The bacteria can be anaerobic or not.

Examples of bacteria encompass, without being limited to: Escherichia coli, Staphylococcus species such as S. aureus, S. epidermis, Pseudomonas such as Pseudomonas aeroginosa, Bacillus, Streptococcus, Bordetella species, Campylobacter, Clostridium, Klebsiella, Legionella, Mycobacterieum, Corynebacterium, Listeria, Mycobacterium, Neisseria, Rickettsia, Salmonella, Shigella, treponema, and Yersinia species. Other examples of bacteria include Bacillus, Bacillota, Bacteroida, Pseudomonadota, Verrucomicrobiota, Actinobacteriota, Fusobacteriota, and Lactobacillus species.

In preferred embodiments, the bacteria is selected from E. coli, Serratia marcescens (S. marcescens) and magnetotactic bacteria (MTB) strains (Magnetospirillum gryphiswaldense strain MSR-1, Magnetospirillum magnetotacticum strain MS-1, Magnetospirillum magneticum strain AMB-1 and Magnetococcus strain MC-1), and Salmonella Typhimurium;
Recombinant versions of said bacteria are also included. The cell can be also a yeast or mold such as those belonging to Candida, Aspergillus, Cryptococcus, Mucorales, Fusarium, Scedosporium, Lomentospora, Blastomyces, Leishmania, Trypanosoma, Saccharomyces (e.g. S. cerevisiae) and Plasmodium species.

Attenuated versions of said bacteria, yeasts or mold, including genetically attenuated ones are also included in the scope of the invention.

When derived from a pluricellular organism, the cell can be either an isolated cell, a cell culture or a cell present in a sample from a tissue or an organ, a cell present in an ex vivo organ or tissue, or a cell present in an organoid. Recombinant cells are also included.

Cells, in particular mammalian cells, can be obtained from cell lines, in particular immortalized cell lines. Examples of cell lines encompass, without being limited to, Chinese hamster ovary (CHO), cancer cell lines such as Hela, MCF-7 and A549, HEK 293, myoblasts cells such as C2C12 cells, dendritic cells such as DC2.5, fibroblasts such as 3T3 cells, lymphocytes such as Jurkat cells, Ptk2 cells, Vero cells, and neuronal cell lines such as SH-SY5Y or SK-N-MC cells.

The cell line can be adherent or able to grow in suspension.

Mammalian cells can be also isolated from a subject such as a human subject, in particular when applications in therapy or diagnosis are sought. The cells can be isolated from peripheral blood, placental blood, umbilical cord blood, amniotic fluid, bone marrow, liver and/or spleen or can be isolated from biopsy performed on various organs. The cell may be differentiated cells of any type. For instance, cells can be erythrocytes also called red blood cells (RBC).

Cells of interest can include all cells involved in immunity such as innate immunity cells (dendritic cells, macrophages, kupffer, NK . . . ) or adaptive immunity cells (B, T lymphocytes . . . ) regardless their origin or whether they are resident in tissues or circulating.

Preferred cells encompass, without being limited to erythrocytes, Hematopoietic Stem cells (HSC), Peripheral Blood Mononuclear cells (PBMC) and in particular T lymphocytes and dendritic cells (DC).

Cells of interest can also be cells derived from engineering manipulations such as Chimeric Antigen Receptor T Cells (CAR-T) or induced pluripotent stem cells (iPSC).

In addition to iPSCs, progenitor cells, whether unipotent, pluripotent, multipotent or totipotent are also encompassed For instance, the cells can be hematopoietic stem cells (homocytoblast), human mesenchymal stem cells (hMSC), endothelial stem cells, tissue stem/progenitor cells (e.g., a neural stem cell, myocyte stem cell or pulmonary stem cell), an umbilical cord stem cell, and the like.

In preferred embodiments, for ethical reasons, the cell is not a human embryonic cell or a cell obtained by a method including the destruction of human embryos. More generally, the method of the Invention is not implemented on human embryonic cells or on cells derived from human embryos by a method including their destruction.

It goes without saying that the method of the Invention is not implemented on a human being. The process of the invention is preferably carried out ex vivo, more preferably in vitro.

As mentioned above, the method of the Invention can be carried out on extracellular vesicles.

In certain embodiments, the extracellular vesicles are obtained from bacteria, in particular from pathogenic bacteria.

In some embodiments, the extracellular vesicles are obtained from eucaryotic cells, in particular from mammal cells such as human cells.

In preferred embodiments, for ethical reasons, the extracellular vesicles do not derive from a human embryonic cell or a human embryo through a method including the destruction of human embryos.

In some particular embodiments, the extracellular vesicle can be isolated from a subject such as a human subject, in particular when applications in therapy or diagnosis are sought.

The extracellular vesicles can be isolated from peripheral blood, placental blood, umbilical cord blood, amniotic fluid, bone marrow, liver and/or spleen or can be isolated from biopsy performed on various organs, in particular from tumour biopsy.

Alternatively, the extracellular vesicles, can be also produced and isolated in vitro from cell culture, in particular from stem cell culture.

In some particular embodiments, the extracellular vesicle is selected from an exosome and an ectosome.

### c) the Chemical Reagent

As mentioned above, the method of the Invention aims at decorating the cell surface with functional moieties of interest. The functional moiety may be of any type.

Accordingly, the chemical reagent used in the method of the Invention is of formula (I) or (IP):

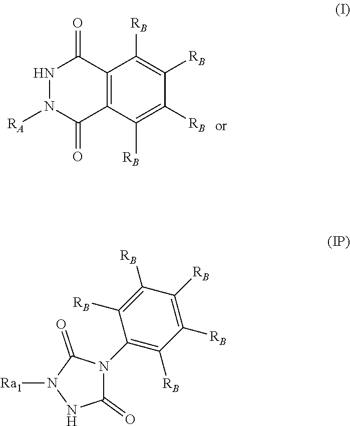

### Wherein:

R_{A} is selected from the group consisting of C₁-C₆ alkyl, a C₆-C₁₄ aryl optionally substituted, a (C₆-C₁₄ aryl)-(C₁-C₃ alkyl) optionally substituted, and -(Y₁)ₙ-M₁.
Rₐ₁ is H or R_{A} as defined above, preferably C₁-C₆ alkyl or H,
each R_{B} is independently a group of formula -(Y₁)ₙ-M₁, a hydrogen or a substituent selected from the group consisting of a halogen, C₁-C₆ alkyl, C₆-C₁₄ aryl, C₃-C₆ cycloalkyl, C₁-C₆ alkoxy, C₁-C₆ alkylamino, C₂-C₆ heterocycle, C₁-C₆ alkanoyl, C₁-C₆ carboxy esters, C₁-C₆ acylamino, —COOH, —CONH₂, —NO₂, —SO₃H, —CN, —CF₃, C₁-C₆ hydroxyalkyl, C₁-C₆ haloalkyl, C₁-C₆ thioalkyl, C₁-C₆ alkylthio, C₂-C₁₀ alkoxyalkyl, and C₂-C₆ alkoxycarbonyloxy,
k is 1 or 2,
n is 0 or 1,
Y₁ is a spacer, and
M₁ is a functional moiety and wherein
when the chemical reagent is of formula (I), at least one group among R_{A} and R_{B} groups is -(Y₁)ₙ-M₁ and when the chemical reagent is of formula (IP), at least one group among Rₐ₁ and R_{B} groups is -(Y₁)ₙ-M₁.

Under the method of the invention, in particular when performed by electrochemistry at low potential (see above), the chemical reagent of formula (I) specifically reacts with accessible tyrosine residues present in surface components, e.g. surface proteins in the cell so as to form a chemically-modified tyrosine of formula (B):

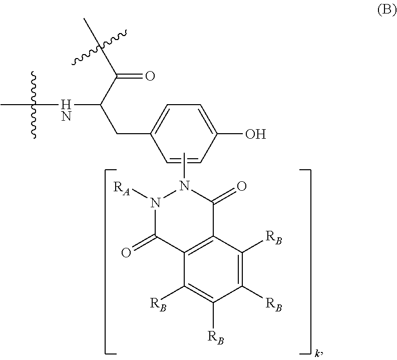

wherein:
R_{A} and R_{B} are as defined in formula (I) (see above and below), and
k is 1 or 2.

Under the method of the invention, in particular when performed by electrochemistry at low potential (see further above), the chemical reagent of formula (IP) specifically reacts with accessible tyrosine residues present in surface components, e.g. surface proteins in the cell so as to form a chemically-modified tyrosine of formula (BP):

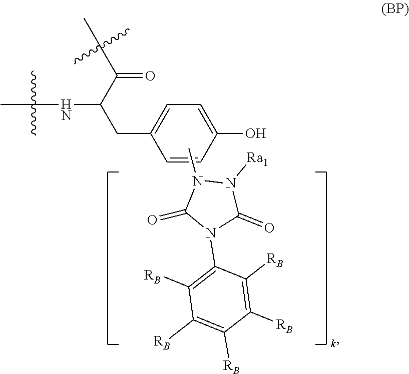

wherein:
Rₐ₁ and R_{B} are as defined in formula (I) (see above and below), and
k is 1 or 2.

In the formulae described in the present application (such as in formula (B) or (BP)), the following moiety represents a tyrosine in a cell surface component, in particular in a cell surface protein:

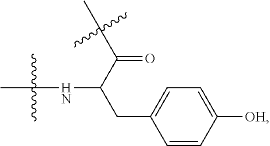 [SMILES: *NC(Cc1ccc(O)cc1)C(*)=O]

wherein

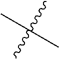

represents a bond by which the tyrosine is attached to the rest of the protein. The N-substituted luminol moiety(ies) can be added at position ortho of the phenol group in the tyrosine residue.

In formula (B) above, when k is 1, the chemically-modified tyrosine residue is modified by one N-substituted luminol derivative of formula (I) and can be represented by the formula (B-1):

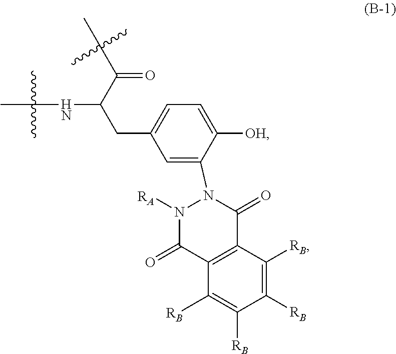

wherein R_{A} and R_{B} are as defined herein.

“k is 2” means that two moieties of formula (II) as defined above is attached to the tyrosine residue. Typically, when k is 2, the at least one chemically-modified tyrosine residue in the capsid can be represented by the formula (B-2):

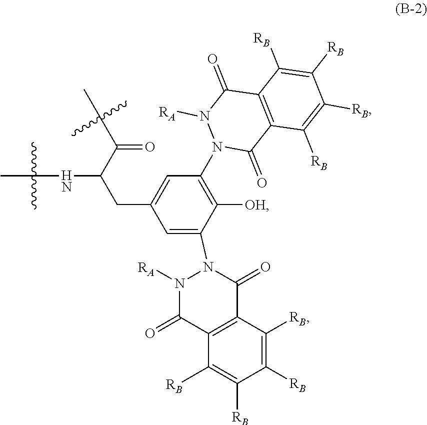

wherein R_{A} and R_{B} are as defined herein.

Similarly, in formula (BP), the chemically-modified tyrosine residue is substituted by one N-substituted PhUr derivative of formula (IP) (k=1) or two N-substituted PhUr derivative of formula (IP) (k=2), said substituents being preferably on position ortho of the phenol group.

The group R_{A} and Rₐ₁

In the above formula (I), R_{A} is a C₁-C₆ alkyl, a C₆-C₁₄ aryl optionally substituted, or a (C₆-C₁₄ aryl)-(C₁-C₃ alkyl) optionally substituted.

In some embodiments, R_{A} is:
a C₁-C₆ alkyl;
a C₆-C₁₄ aryl optionally substituted by one or more (preferably one) groups chosen from halogen, C₁-C₆ alkyl, C₆-C₁₄ aryl, C₃-C₆ cycloalkyl, C₁-C₆ alkoxy, C₁-C₆ alkylamino, C₂-C₆ heterocycle, C₁-C₆ alkanoyl, C₁-C₆ carboxy esters, C₁-C₆ acylamino, —CF₃, C₁-C₆ hydroxyalkyl, C₁-C₆ haloalkyl, C₁-C₆ alkylthio, C₁-C₆ thioalkyl, C₂-C₁₀ alkoxyalkyl, C₂-C₆ alkoxycarbonyloxy and (C₆-C₁₄ aryl)-(C₁-C₃ alkyl);
a (C₆-C₁₄ aryl)-(C₁-C₃ alkyl) optionally substituted by one or more (preferably one) groups chosen from halogen, C₁-C₆ alkyl, C₆-C₁₄ aryl, C₃-C₆ cycloalkyl, C₁-C₆ alkoxy, C₁-C₆ alkylamino, C₂-C₆ heterocycle, C₁-C₆ alkanoyl, C₁-C₆ carboxy esters, C₁-C₆ acylamino, —CF₃, C₁-C₆ hydroxyalkyl, C₁-C₆ haloalkyl, C₁-C₆ alkylthio, C₁-C₆ thioalkyl, C₂-C₁₀ alkoxyalkyl, C₂-C₆ alkoxycarbonyloxy and (C₆-C₁₄ aryl)-(C₁-C₃ alkyl), or
-(Y₁)ₙ-M₁ (defined further below)

Preferably, the substituents are selected from the group consisting of halogens, C₁-C₃ alkyl, C₁-C₃ alkoxy, C₁-C₃ hydroxyalkyl, and C₁-C₃ haloalkyl.

In a preferred embodiment, R_{A} is a C₁-C₃ alkyl (such as methyl), a phenyl, or a benzyl. More preferably, R_{A} is a methyl or a benzyl. Even more preferably, R_{A} is a methyl.

In formula (IP), Rₐ₁ is H or has the same definition as R_{A}.

### The Group R_{B}

In the above formula (I) or (IP), each R_{B} is independently selected from a group of formula -(Y₁)ₙ-M₁ (wherein Y₁, n and M₁ are as defined further below), a hydrogen or a substituent chosen from a halogen, C₁-C₆ alkyl, C₆-C₁₄ aryl, C₃-C₆ cycloalkyl, C₁-C₆ alkoxy, C₁-C₆ alkylamino, C₂-C₆ heterocycle, C₁-C₆ alkanoyl, C₁-C₆ carboxy esters, C₁-C₆ acylamino, —COOH, —CONH₂, —NO₂, —SO₃H, —CN, —CF₃, C₁-C₆ hydroxyalkyl, C₁-C₆ haloalkyl, C₁-C₆ alkylthio, C₁-C₆ thioalkyl, C₂-C₁₀ alkoxyalkyl, and C₂-C₆ alkoxycarbonyloxy,
with proviso that when the chemical reagent is of formula (I), at least one group among R_{A} and R_{B} groups is -(Y₁)ₙ-M₁ and when the chemical reagent is of formula (IP), at least one group among Rₐ₁ and R_{B} groups is -(Y₁)ₙ-M₁.

In some embodiments, the chemical reagent of formula (I) or (IP) is such that R_{A} or Rₐ₁ is -(Y₁)ₙ-M₁ respectively. Preferably, none of R_{B} groups is -(Y₁)ₙ-M₁. In particular all R_{B} groups are H.

In preferred embodiments, the chemical reagent of formula (I) or (IP) is such that one or two (preferably one) R_{B} is a group of formula -(Y₁)ₙ-M₁.

More preferably, the chemical reagent of formula (I) or (IP) is such that one or two (preferably one) R_{B} is a group of formula -(Y₁)ₙ-M₁, and the other R_{B} are hydrogens.

-(Y₁)ₙ-M₁ can be at any position of the aromatic ring (namely, any R_{B}).

Additionally, R_{A} or Rₐ₁ are not -(Y₁)ₙ-M₁.

In preferred embodiments, the chemical reagent is of formula (I).

Preferably, the chemical reagent of formula (I) as defined above is of formula (I-a) or (I-b).

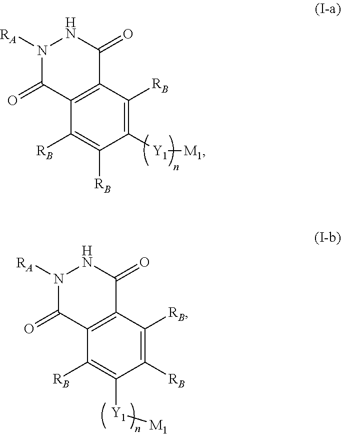

wherein each R_{B} is independently a hydrogen or a substituent chosen from a halogen, C₁-C₆ alkyl, C₆-C₁₄ aryl, C₃-C₆ cycloalkyl, C₁-C₆ alkoxy, C₁-C₆ alkylamino, C₂-C₆ heterocycle, C₁-C₆ alkanoyl, C₁-C₆ carboxy esters, C₁-C₆ acylamino, —COOH, —CONH₂, —NO₂, —SO₃H, —CN, —CF₃, C₁-C₆ hydroxyalkyl, C₁-C₆ haloalkyl, C₁-C₆ alkylthio, C₁-C₆ thioalkyl, C₂-C₁₀ alkoxyalkyl, and C₂-C₆ alkoxycarbonyloxy, preferably each R_{B} is a hydrogen.

In a particular embodiment, the chemical reagent is of formula (I-c)

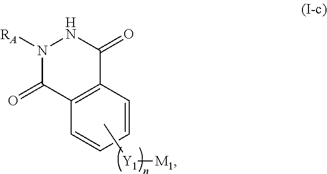

in particular

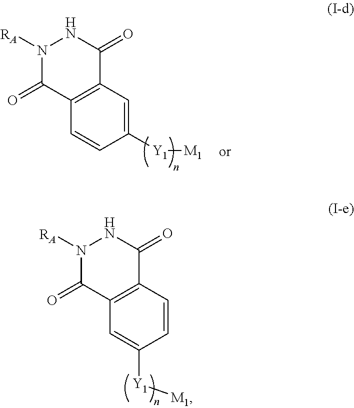

R_{A} being as defined above. Preferably, R_{A} is a C₁-C₃ alkyl, a phenyl optionally substituted, or a benzyl optionally substituted. More preferably, R_{A} is a methyl, phenyl or a benzyl.
Even more preferably, R_{A} is a methyl, namely a chemical reagent of formula (I-f) or (I-g)

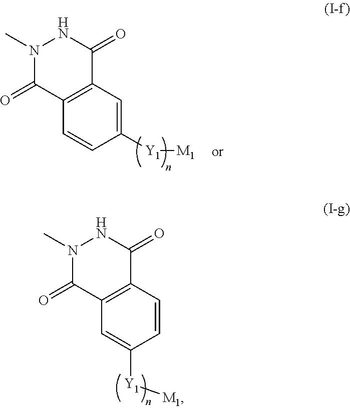

In some other embodiments, the chemical reagent is of formula (IP), preferably of formula (IPa).

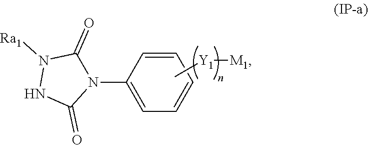

Wherein Rₐ₁ is preferably H or —CH₃.

In other embodiments, the chemical reagent is of formula (I-h) or (IP-b) as follows

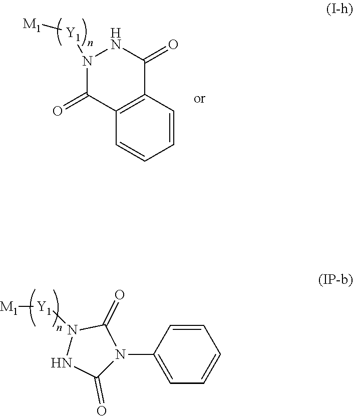

The -(Y₁)ₙ-M₁ Moiety

As mentioned above, Y₁ refers to a spacer group and M₁ to a functional group to be coupled at the cell surface.

Y₁ is a spacer that links the N-substituted luminol moiety and the functional moiety M₁ together. Y₁ may be present (when n is 1) or absent (when n is 0). When Y1 is absent, the N-substituted luminol moiety and M₁ are directly linked to each other. Accordingly, n is either 0 or 1.

The functional moiety “M₁” may be of any type. “M₁” is typically selected depending on the biological effect which is sought by introducing M₁ on cell surface.

Alternatively, M₁ can be a reactive group selected so as to enable a subsequent step of coupling.

Accordingly, “M₁” may comprise a moiety selected from a chemical reactive group enabling biorthogonal reaction such as a click-chemistry reactive group, a targeting agent, a steric shielding agent, a labelling agent, an oligonucleotide, a carbohydrate, or a drug such as antibiotic, anticancer drugs such as cytotoxic drug, antitumoral, or anti-angiogenesis drugs, a ligand, a polypeptide, a peptide, a hormone and the like.

For instance, M₁ can be a drug selected from a VEGFR inhibitor, an EGFR TK inhibitor, a PIK-1 modulator, a Bcl-2 inhibitor, an HDAC inhibitor, a PARP inhibitor, a Cdk inhibitor, a PI3 kinase inhibitors, a JAK/STAT inhibitor, an immune checkpoint-inhibitor, a focal a Map kinase kinase (mek) inhibitor, Topoisomerase inhibitors, alkylating agents, anti-microtubule agents, and the like. For illustration, the drug can be an anti-PD1 antibody, anticancer small drug such as tamoxifen, imatinib, carboplatin, cisplatin, ifosfamide, cyclophosphamide, methotrexate, fludarabine, 5-fluorouracile, vinblastine, daunorubicine, idarubicine, doxorubicine, Monomethyl auristatin E (MMAE) and the like

“M₁” may be also a (nano)-particle, including a magnetic (nano-) particle, a quantum dot and liposomes, including liposomes encapsulating drugs or imaging agents. For instance, M₁ may be an iron, stain, silicium, gold or carbon (nano)-particle or a phospholipid-based liposome.

In some embodiments, M₁ may be a labelling moiety such as a radionuclide, a fluorophore or any agent enabling imaging such as chelator agents of lanthanides or other chemical species. For instance for imaging purpose, e.g., for positron emission tomography, or for therapeutic purpose (e.g. anti-cancer therapy with radionuclides), M₁ can be a chelator such as a macrocyclic polyamino carboxylate (e.g., DOTA and TETA), or an acyclic chelating agent (e.g., EDTA and DTPA). The chelator may bind or be specific for ¹⁷⁷Lu, ⁶⁴Cu, ⁸⁶Y, ⁶⁸Ga, ⁸⁹Zr, or ^{94m}Tc, and for MRI, M1 may include a chelator of lanthanides (such as gadolinium) e.g., a hydroxypyridonate, DTPA, HOPO, TR322, TR332, TPPN, HP-DO3A, DO3A-butrol, DTPA-BMA, DTPA-BMEA, BOPTA, EOB-DPTA, MS-325 or DOTA.

In some other embodiments, M₁ can be a molecule, such as drug, comprising a radionuclide such as ¹³¹I, ⁹⁰Y, ¹⁷⁷Lu, ⁶⁷Cu, ¹⁸⁶Re, ¹⁸⁸Re, ²¹¹At, e.g. for providing anti-cancer, in particular anti-tumor, therapy.

In other embodiments, “M₁” comprises, or consists of, a steric shielding agent, e.g. an agent able to mask certain epitopes present on the cell surface. For instance, “M₁” may be a polyethylene glycol (PEG), pHPMA or a polysaccharide.

“M₁” may be also an oligonucleotide such as messenger RNA (mRNa) or antisense oligonucleotides such as small interferent RNA (siRNA), shRNA, snoRNA and meroduplex (mdRNA) but also a nucleic ligand such as an aptamer

For instance, “M₁” may be a ligand which specifically binds to a membrane biological entity (e.g. a membrane receptor). Said ligand may be of any type e.g. a peptide a protein, an oligosaccharide or a small chemical entity. For instance, M1 may be a mono- or a polysaccharide, a hormone, including a steroid hormone, a peptide, an aptamer, an antibody including heavy-chain antibody, and fragments thereof such as Fab, Fab′, and VHH (also called nanobody), a ScFv, a spiegelmer, a peptide aptamer, biotin, (strept)avidin, cation binding groups, protein tags (e.g. c-myc tag, hemaglutinin antigen (HA) tag, thioredoxin tag, FLAG tag, polyArg tag, polyHis tag, Strep-tag) and the like.

In some embodiments, “M₁” comprises, or consists of, a cell-type specific ligand derived from proteins such as transferrin, Epidermal Growth Factor (EGF), and basic Fibroblast Growth Factor βFGF. In some other embodiments, “M₁” comprises, or consists of, a cell-type specific ligand derived from mono- or polysaccharides, e.g. comprising one or several galactose, mannose, mannose-6-phosphate, N-acetylgalactosamine (GalNac) and bridged GalNac and sialic acid and derivatives thereof (such as Neu5Ac, Neu5Acα2-6Gal, Neu5Acα2-8Neu5Ac). The mono- or polysaccharides can be natural or synthetic. In another embodiment, “M₁” comprises, or consists of, a cell-type specific ligand derived from vitamins such as folic acid.

Y₁ may be any chemical chain (e.g. hydrocarbon chain) which can comprise heteroatoms as well as cyclic moieties such as cycloalkyl, cycloalkenyl, aromatic groups, or heterocyclic moieties such as heterocycloalkyl or heteroaryl. Y₁ may comprise up to 1000 carbon atoms and even more.

The length and the chemical nature of the spacer may be optimized depending on the functional moiety “M₁” which is intended to be coupled on the cell surface and the biological effect which is sought.

Indeed, further to its linking function, Y₁ may be used to refine the properties of the functional moiety “M₁”. For instance, Y₁ may decrease the steric hindrance of M₁ with respect to the cell surface, improve the accessibility of M₁ for binding with a biological entity of interest, improve the binding of M₁ with an entity of interest and/or increase the solubility of the chemical reagent.

In some embodiments, Y₁ is a chemical chain group comprising from 2 to 1000 carbon atoms, preferably from 2 to 500 carbon atoms, from 2 to 300 carbon atoms, e.g. from 2 to 100 carbon atoms, 2 to 40 carbon atoms, from 4 to 30 carbon atoms or from 4 to 20 carbon atoms.

Typically, Y₁ is selected from the group consisting of polymers including homopolymers, copolymers and block polymers, peptides, oligosaccharides, and saturated or unsaturated hydrocarbon chains optionally interrupted by one or several heteroatomic groups (e.g. S, O, Se, P, —C(O)—, —NHC(O)—, —OC(O)—, —N(R)— with R being H or C₁-C₃ alkyl), and/or by one or several cyclic or heterocyclic moieties, and/or optionally having an heteroatomic group (such as S, O, —C(O)—, —NHC(O)—, —OC(O)—, —N(R)— with R being H or C₁-C₃ alkyl, —O—N(R)— with R being H or C₁-C₃ alkyl, or —N(C₁-C₃ alkoxy)-) at least one of its extremity, and/or optionally being substituted by one or several substituents (e.g. hydroxyl, halogens, C₁-C₃ alkoxy, —CN, —CF₃, or C₁-C₃ alkyl), and combinations thereof.

As used herein, “combinations” means that the spacer group Y may comprise several hydrocarbon chains, oligomer chains or polymeric chains (e.g. 2, 3, 4, 5 or 6) linked (or connected) by any appropriate group, such as —O—, —S—, —N(R)— with R being H or C₁-C₃ alkyl, —C(O)—, —NHC(O)—, —OC(O)—, —C(O)—O—C(O)—, —NH—CO—NH—, —O—CO—NH—, NH—(CS)—NH—, NH—CS—, phosphodiester or phosphorothioate groups as well as cyclic or heterocyclic groups.

Typically, the group(s) (also called connectors) used to link the several hydrocarbon chains, oligomer chains or polymeric chains together result from the reactions used to connect these different chains together. For instance, the connector may be —NHC(O)— in case of amide coupling reaction, “N” in case of reductive amination or a triazole derivative in case of click chemistry involving the reaction of an azido with an alkyne group.

In some embodiments, Y₁ may be selected from the group consisting of polyethers such as polyethylene glycol (PEG) and polypropylene glycol, polyvinyl alcohol (PVA), polyesters such as polylactate, polyacrylate, polymethacrylate, polysilicone, polyamide such as polycaprolactone and poly(N-(2-hydroxypropyl)methacrylamide) (pHPMA), poly(D,L-lactic-co-glycolic acid) (PLGA), polymers of alkyl diamines, unsaturated or saturated, branched or unbranched, hydrocarbon chains optionally having an heteroatom such as 0, NH and S on at least one end, and combinations thereof.

As used herein, alkyl diamine refers to NH₂—(CH₂)ᵣ—NH₂ with r is an integer from 2 to 20, for instance from 2 to 10 such as 2, 3, 4, and 5. A polymer of alkyl diamines (also known as polyamines) refers to a compound of formula NH₂—[(CH₂)ᵣ—NH]ₜ—H with r being as defined above and t is an integer of at least 2, for example of at least 3, 4, 5, 10 or more. Polymers of alkyl diamines of interest are, for instance, spermidine, and spermine.

For instance, Y₁ can comprise at least one polyethylene glycol moiety comprising from 2 to 40 monomers, e.g. from 2 to 10 or 2 to 6 monomers. For illustration only, Y1 may comprise from 2 to 10 triethyleneglycol blocks linked together by linkers. As another example, Y1 may be a C₁₂ hydrophilic triethylene glycol ethylamine derivative. Alternatively, Y1 may be a saturated or unsaturated C₂-C₄₀ hydrocarbon chain, in particular a C₁₀-C₂₀ alkyl chain or a C₂-C₁₀ alkyl chain such as a C₆ alkyl chain. The alkyl chain may have a group such as NH, S or O on at least one end. For instance, Y1 may be putrescine.

In a particular embodiment, Y₁ is selected from the group consisting of saturated or unsaturated, linear or branched C₂-C₄₀ hydrocarbon chains, optionally substituted, polyethylene glycol, polypropylene glycol, pHPMA, PLGA, polymers of alkyl diamines and combinations thereof.

In a particular embodiment, Y₁ is selected from the group consisting of linear or branched C₂-C₂₀ alkylene chains, polyethylene glycol, polypropylene glycol, pHPMA, PLGA, polymers of diamino alkyl and combinations thereof. Preferably said polyethylene glycol, polypropylene glycol, PLGA, pHPMA and polymer of alkyl diamines comprise from 2 to 40 monomers, preferably from 2 to 10 or from 10 to 20 monomers.

For instance, Y₁ may comprise one or several (e.g. 2, 3, 4 or 5) triethylene glycol blocks.

In some embodiments, Y₁ is a hydrocarbon chain (for instance an alkyl chain) having from 2 to 100 carbon atoms, 2 to 40 carbon atoms, from 2 to 30 carbon atoms, or from 2 to 20 carbon atoms, optionally interrupted by:
one or more heteroatomic groups chosen from —O—, —S—, —C(O)—, —NHC(O)—, —OC(O)—, —C(O)—O—C(O)—, —N(R)— with R being H or a C₁-C₃ alkyl, —NH—CO—NH—, —O—CO—NH—, NH—(CS)—NH—, and —NH—CS—; and/or
C₅-C₂₀ carbocyclic moieties such as cycloalkyl, cycloalkenyl, or aromatic groups; and/or
one or more heterocyclic moieties having 5 to 20 ring atoms, such as heterocycloalkyls or heteroaryls having 5 to 20 ring atoms;
   and optionally having at least one of its extremities, an heteroatomic group chosen from —O—, —S—, —N(R)— with R being H or C₁-C₃ alkyl, —O—N(R)— with R being H or C₁-C₃ alkyl, —N(C₁-C₃ alkoxy), —C(O)—, —NHC(O)—, —OC(O)—, —C(O)—O—C(O)—, —NH—, —NH—CO—NH—, —O—CO—NH—, NH—(CS)—NH—, NH—CS—.

In some embodiments, Y₁ is a hydrocarbon chain (for instance an alkyl chain) having from 2 to 100 carbon atoms, 2 to 40 carbon atoms, from 2 to 30 carbon atoms, or from 2 to 20 carbon atoms, optionally interrupted by:
one or more heteroatomic groups chosen from —O—, —S—, —N(R)— with R being H or a C₁-C₃ alkyl, —C(O)—, —NHC(O)—, and —OC(O)—; and/or
one or more heterocyclic moieties having 5 to 20 ring atoms, such as heterocycloalkyls or heteroaryls having 5 to 20 ring atoms;
   and optionally having an heteroatomic group chosen from —O—, —S—, —N(R)— with R being H or C₁-C₃ alkyl, —O—N(R)— with R being H or C₁-C₃ alkyl, —N(C₁-C₃ alkoxy)-, —C(O)—, —NHC(O)—, and —OC(O)— at least one of its extremities.

For instance Y₁, may be a C₂-C₁₀, such as C₂-C₆ alkyl chain.

In some embodiments, Y₁ can comprise a cleavable group able to release M₁, e.g. in specific conditions. For instance, Y₁ can be an enzymatically cleavable linker or a linker which can be cleaved by chemical reaction with a chemical partner. For instance Y₁ can comprise a trans-cyclooctene (TCO) moiety on which is coupled M1 e.g. through —OCO—NH— linker which can release M1 through a click reaction with a tetrazine molecule (i.e. by the so-called “click and release” reaction described by Robillard's group—See for instance the review in that matter from Ji et al., Chem. Soc. Rev. 2019, 48,1077-1094). The use of a cleavable linker can be of interest when M₁ corresponds to a drug, preferably an anti-tumoral drug so as to enable the targeted release of the drug in a specific site (e.g. a tumor site) in vivo.

Y₁ and M₁ present in the chemical reagent of the invention are typically selected so as to be compatible with the conditions of the incubation step (e.g. electrochemical or enzymatic incubation) described above. In other words, the functional group M₁ or the spacer group Y₁ in the chemical reagent should not be oxidized under the oxidation conditions of the incubation step or should not react with the activated N-substituted luminol or N-substituted PhUr. Preferably, the functional group M₁ of the chemical reagent does not comprise any phenol moiety. In other embodiments, M₁ and Y₁ do not contain any alkene or alkyne moieties.

More generally, the group M₁ and the possible Y₁ present in the chemical reagent of formula (I) and (Ia) to (Ig) may not contain any chemical group having an oxidation potential equal or lower than that of the N-substituted luminol moiety.

More generally, the group M₁ and the possible Y₁ present in the chemical reagent of formula (IP) and (IPa) may not contain any chemical group having an oxidation potential substantially equal (e.g. ±150 mV) or lower than that of the N-substituted PhUr moiety.

The introduction of a functional group having a chemical group which is not compatible with electrochemical activation as described above can be performed in two steps namely: the introduction of a chemical moiety on the cell surface able to undergo a click chemistry reaction, and then the introduction of the functional moiety of interest having such an incompatible chemical moiety through click chemistry.

In a particular embodiment M₁ is a chemical reactive group, more preferably a “biocompatible chemical reactive group”. As used herein, M₁ can enable to create a covalent interaction between the cell surface and an entity of interest, without significantly altering the functionality of the cell (and thus in a biocompatible way). In other words, the functional moiety may comprise a chemical reactive group which can promote the formation of a covalent bond with an entity of interest so as to covalently link it at the surface of the cell. For instance, the functional moiety may comprise a chemical reactive group suitable to create a covalent bond by click-chemistry or by bioconjugation reaction. Bioconjugation reactions encompass reactions between amino acids such as lysine, cysteine or tyrosine with reactive groups as detailed in Koniev, O., Wagner, A, Chem. Soc. Rev., 44, 5495 (2015).

Preferably, M₁ is a click-chemistry reactive group, also called hereunder a “click-chemistry group”.

As used herein, a “click-chemistry group” refers to any reactive chemical group that can be involved in a click chemistry reaction. Preferably, M₁ is not a thiol (—SH).

“Click-reaction” or “Click-chemistry” is a concept introduced by Sharpless in 2001. “Click chemistry” generally refers to chemical reactions characterized by high yields, high chemoselectivity, which are simple to conduct and which generate inoffensive by-products. “Click reactions” can be typically conducted in complex media with high efficiency. Click reactions are typically used to create covalent heteroatom links (C—X—C) between two entities of interest. For review about click chemistry, one can refer to Kolb et al., Angew. Chem. Int. Ed. 2001, 40, 2004-2021) and to Rudolf et al., Current opinion in Chemical Biology, 2013, 17:110-117.

Examples of click chemistry reactions include, but are not limited to, Staudinger Ligation, azido-ene or azido-alkyne click-chemistry, carbonyl condensation, sydnone-alkyne cycloaddition, tetrazole-ene reaction, nitrile oxide-ene click chemistry, nitrile imine-ene click chemistry, inverse electron demand Diels-Alder ligation, isonitrile-tetrazine click chemistry, Suzuki-Miyaura coupling. Preferably, the click chemistry reaction is not thiol-ene or thiol-maleimide reaction.

For instance, M₁ may comprise, or consist of, an azido (—N₃), phosphine such as a functionalized triarylphosphine, aldehyde, ketone, hydrazide, oxyamine, nitrile oxide, oxime, hydroxymoyl chloride, chlororoxime, nitrile imine, hydrazone, hydrazonoyl chloride, chlorohydrazone, tetrazine, tetrazole isonitrile, aryl halide, aryl boronate, oligo-histidine, nickel-complex or nickel ligand. In a preferred embodiment, M1 is N₃.

### d) Additional Steps and Particular Embodiments

The method of the invention may comprise one or several additional steps prior to, or after the step of incubation (e.g. electrochemical or enzymatic incubation) as described above.

For instance, the method of the invention may comprise a step of providing or producing the cell entities (e.g. the cells) to be chemically modified.

The method of the invention may also comprise a step of providing or preparing the chemical reagent.

The chemical reagent can be produced by synthetic routes as illustrated in the example section.

When the electrochemical conditions described above are used, the method of the Invention can also comprise a step of determining the oxidation potential of the chemical reagent versus a reference electrode such as Ag/AgCl in saturated KCl or saturated calomel electrode (SCE).

Before being incubated with the chemical reagent, the cells can be subjected to a pre-treatment, such as trypsin treatment (e.g. in case of cell culture) and/or centrifugation and/or resuspension in an appropriate buffer suitable for performing the incubation step.

The method of the invention may also comprise one or several additional steps following the step of incubation, such as:
a step of removing the unreacted reagent, e.g. by centrifugation and/or
a step of resuspending the cells in an appropriate medium and/or
a step of collecting the chemically modified cellular entities (preferably the cells) and/or
a step of freezing the cellular entities (preferably the cells) for storage.

When M₁ present in the chemical reagent of formula (I) or (Ia) to (Ig), (IP) or (IPa), (preferably (I) or (Ia) to (Ig)) is a reactive chemical group for bioconjugation or click-chemistry group, the cellular entity (preferably the cell) which has been chemically modified with the chemical reagent can undergo a supplementary step aiming at coupling a functional group M₂ through a reaction with M1 group. This supplementary step is particularly suitable when M₂ is a functional group incompatible with the conditions of the incubation step (e.g. electrochemical or enzymatic incubation) described above. The incompatibility of M₂ to electrochemistry may for instance be due to the ability of M₂ to be oxidized under the oxidation conditions (i.e. M₂ comprises a functional group having a oxidation potential lower than or equal to that of the N-substituted luminol or the N-substituted PhUr) of the incubation step or its ability to react with the activated N-substituted luminol or PhUr.

M₂ may for instance be a functional group comprising a phenol moiety or other moieties that can be found in certain fluorophores incompatible with electrochemistry. It is the case, for instance, of the fluorophores used in the Example section such as carboxy rhodamine or carboxy fluorescein.

Other examples are xanthene derivatives such as fluorescein and rhodamine, Cyanines 3/5/7 (Cy3, Cy5, Cy7), and peptides/polypeptides comprising tyrosine residues e.g. therapeutic antibodies and therapeutic proteins. More generally, the below two step methods are preferably implemented for M₂ being a fluorophore and/or a polypeptide containing tyrosine residues (for instance antibodies or an antigen-binding domain derived thereof, cytokine, grow factors, enzymes . . . ) and/or a drug comprising a phenol group which does not bear any electro-attractive substituent such as topotecan.

In some further embodiments, the below two step methods are also preferred when the functional group to introduce (i.e. M₂) comprises unsaturation(s) (i.e. alkyne or alkene moieties).

Accordingly, the Invention also relates to a method for decorating the surface of a cellular entity (preferably a cell) with a functional moiety M₂ (called hereunder Embodiment 1), said method comprising the steps of:
i. chemically modifying the surface of the cellular entity (preferably the cell) with a chemical reagent bearing a N-substituted luminol moiety of formula (I) or (Ia)-(Ih) or a N-substituted PhUr derivative of formula (IP) (IP-a) or (IP-b) wherein M₁ is a click-chemistry group, preferably by the method as described above in particular by electrochemistry, and
ii. Immobilizing the functional group M₂ at the cellular entity surface by promoting a click reaction with M₁.

In such embodiment, M₁ present in the chemical reagent used in step (i) is a click-chemistry group compatible with the conditions of the incubation step (e.g. electrochemical or enzymatic incubation) described above, namely a click chemistry group that cannot be oxidized under the oxidation conditions (i.e. having an oxidation potential higher than that of the N-substituted luminol or the N-substituted PhUr) of the incubation step or that cannot react with the activated N-substituted luminol.

Preferably, in such embodiment, M₁ is an azido, phosphine, aldehyde, ketone, hydrazide, oxyamine, nitrile oxide, oxime, hydroxymoyl chloride, chlororoxime, nitrile imine, hydrazone, hydrazonoyl chloride, chlorohydrazone, tetrazine, isonitrile, aryl halide, aryl boronate, oligo-histidine, nickel-complex or nickel ligand. More preferably, in such embodiment, M₁ is an azido.

Preferably the chemical reagent is of formula (I) or (Ia)-(Ih).

Typically, in step (ii), the cellular entity (preferably the cell) having its surface chemically modified obtained in step (i) is incubated with a compound of the following formula (III), in condition conducive to the reaction of Q with M₁:

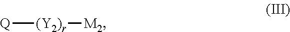

wherein:
   Q is a click-chemistry group that is able to react with M₁ through a click chemistry reaction,
   r is 0 or 1,
   Y₂ is a spacer, and
   M₂ is the functional group to be immobilized on the surface of the cell.
Y₂ is typically as defined above for Y₁
Q is selected so as to specifically react with M₁

M₂ has typically the broadest definition provided for M₁ hereabove except that M2 is not a click-reaction group, but without any restriction with respect to the presence of a phenol group. For instance, M₂ can comprise a moiety selected from a targeting agent, a steric shielding agent, a labelling agent, an oligonucleotide, a drug such as a cytotoxic drug or an antitumoral drug, a ligand, a polypeptide, a peptide, a hormone, a (nano)-particle, including a magnetic (nano-) particle, a liposome and a quantum dot and the like.

Depending on M₁ present in the chemical reagent used in step (i), Q may comprise, or consist of, an azido (—N₃), an alkene, an alkyne (in particular a strained alkyne, such as cyclooctyne (OCT), aryl-less cyclooctyne (ALO), monofluorocyclooctyne (MOFO), difluorocyclooctyne (DIFO), dibenzocyclooctyne (DIBO), dimethoxyazacyclooctyne (DIMAC), biarylazacyclooctynone (BARAC), bicyclononyne (BCN), tetramethylthiepinium (TMTI, TMTH), difluorobenzocyclooctyne (DIFBO), oxa-dibenzocyclooctyne (ODIBO), carboxymethylmonobenzocyclooctyne (COMBO), or benzocyclononyne), phosphine, aldehyde, ketone, hydrazide, oxyamine, nitrile oxide, oxime, hydroxymoyl chloride, chlororoxime, nitrile imine, hydrazone, hydrazonoyl chloride, chlorohydrazone, tetrazine, isonitrile, aryl halide, aryl boronate, oligo-histidine, nickel-complex or nickel ligand.

Examples of complementary click-chemistry groups and click chemistry reactions include, but are not limited to: azido-alkyne click-chemistry (M1=azide and Q=alkyne (e.g. strained intracyclic alkyne)), Staudinger Ligation (M1=azide and Q=phosphine), carbonyl condensation (M1=aldehyde or ketone and Q=hydrazide or oxyamine), sydnone-alkyne cycloaddition (M1=sydnone and Q=alkyne), tetrazole-ene reaction (M1=tetrazole and Q=alkene), nitrile-oxide-ene click chemistry (M1=nitrile oxide or aldehyde, oxime, or hydroxymoyl chloride or chlororoxime and Q=alkene or alkyne), nitrile imine-ene click chemistry (M1=nitrile imine or aldehyde, hydrazone, hydrazonoyl chloride or chlorohydrazone and Q=alkene or alkyne), inverse electron demand Diels-Aider ligation (M1=alkene and Q=tetrazine), isonitrile-tetrazine click chemistry (M1=isonitrile and Q=tetrazine), Suzuki-Miyaura coupling (M1=aryl halide and Q=aryl boronate).

In the above-mentioned listing of click-chemistry groups involved in the click chemistry reactions, M₁ and Q can be permuted, with the proviso that M1 is a click-chemistry group compatible with the conditions of the incubation step (e.g. electrochemical or enzymatic incubation) described above. All the above-mentioned chemical reactions result in a covalent link.

Preferably, M₁ and Q are not thiol group (—SH), and the click chemistry reaction is not thiol-ene or thiol-maleimide reaction.

In a particular embodiment, M₁ is an azido (—N₃) and Q is an alkyne (such as a —C≡CH group or a strained alkyne such as those mentioned above).

In some aspects, the click reaction between M₁ and Q may be “bioorthogonal” and “biocompatible”, this means that M₁ and Q may react selectively and rapidly with each other without side reactions with other entities and without significantly altering the functionality of the cell.

Preferred click reactions are thus those which do not encompass metal catalysts. Such reactions are called metal-free click-reactions and encompass, for instance:

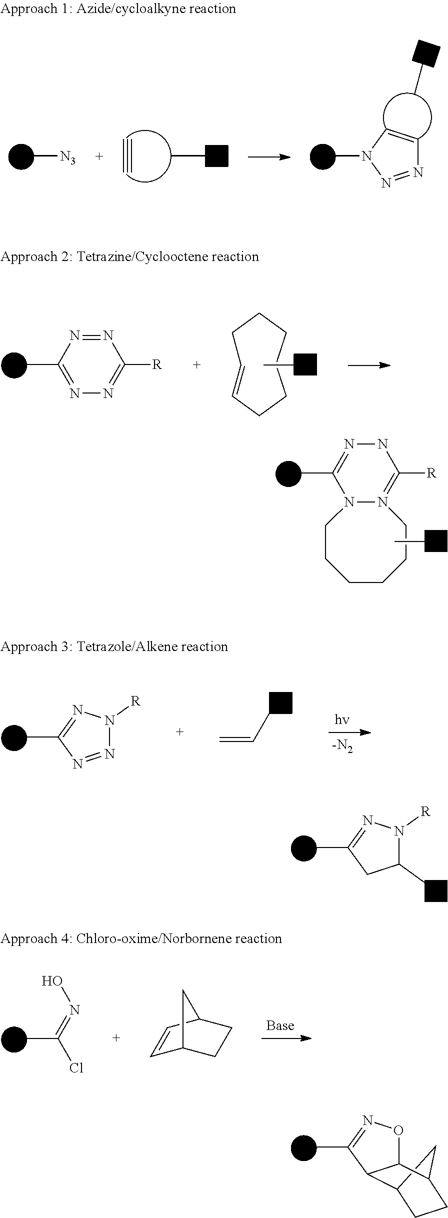

In a particular embodiment, the click reaction of interest is a strain promoted alkyne-azide cycloaddition (SPAAC), which means that M1 can be an azido group and Q can be a strained alkyne as described above. The reaction of M1 and Q results in the formation of a triazolyl fused to another cycle. In step (ii), the entity “-(Y₁)ₙ-T-(Y₂)ᵣ” is form wherein T is a triazolyl optionally fused to another cycle Depending on the nature of Q, the triazolyl fused to another cycle (T) can be one of the following:

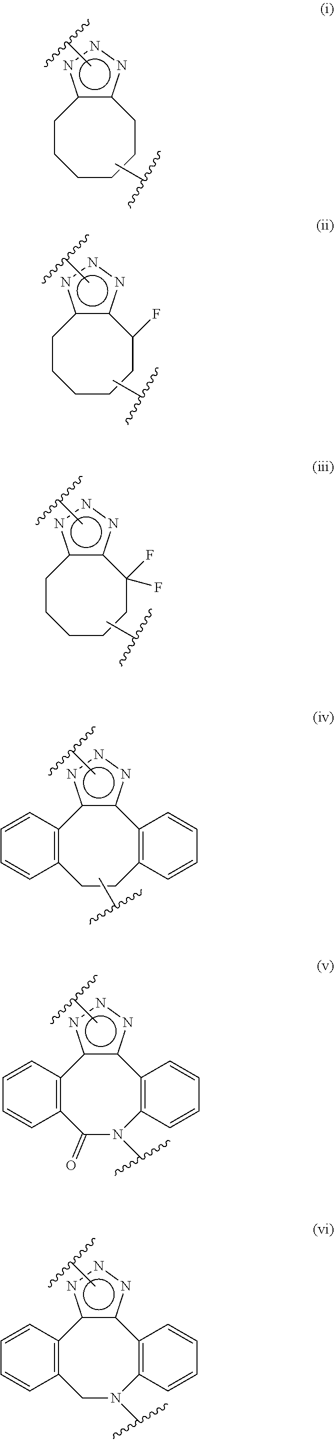

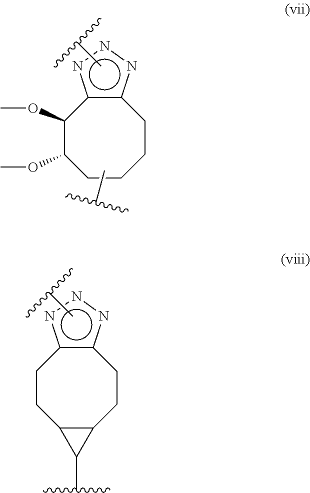

The symbol

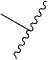

represents the bond by which T is linked to the rest of the moiety.

In another embodiments, the click reaction of interest is a Diels Alder reaction between M₁ which is a tetrazine and Q which is a trans-cyclooctene.

Other strategies can be contemplated to decorate the cell with a functional M₂, in particular when M₂ is not compatible with electrochemistry and/or with click chemistry. One can use specific binding partners to immobilize M₂ instead of creating a covalent bond by click chemistry. In other M₂ is immobilized on the surface of the cell through specific non-covalent interactions between M₁ and Q.

Accordingly, the invention also relates to a method for decorating the surface of a cellular entity (preferably a cell) with a functional moiety M₂ (called hereunder Embodiment 2), said method comprising the steps of:
i. chemically modifying the surface of the cellular entity (preferably the cell) with a chemical reagent bearing a N-substituted luminol moiety of formula (I) or (Ia)-(Ih) or a N-substituted PhUr derivative of formula (IP) (IPa) or (IPb), preferably by the method as described above in particular by electrochemistry, and
ii. incubating the cell having its surface chemically modified obtained in step (i) with a compound of the following formula (III):

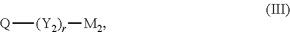

in conditions conductive to promote specific non-covalent interaction between Q and M1.
   r is 0 or 1,
   Y₂ is a spacer, and
   M₂ is the functional group to be immobilized on the surface of the cell.
Y₂ is typically as defined above for Y₁

Preferably the chemical reagent is of formula (I) or (Ia)-(Ig).

M₂ has typically the broader definition provided for M₁ but preferably M₂ is not a click-reaction group. For instance, M₂ can comprise a moiety selected from a targeting agent, a steric shielding agent, a labelling agent, an oligonucleotide, a drug such as a cytotoxic drug or an antitumoral drug, a ligand, a polypeptide, a peptide, a hormone, a (nano)-particle, including a magnetic (nano-) particle and a quantum dot and the like.

M₁ and Q are selected so as to specifically interact together and form a stable complex.

The interaction between M1 and Q may rely on various interaction systems such as affinity systems, ligand/anti-ligand couples or protein tags.

According to the type of interaction sought between M₁ and Q, M₁ may comprise, for example, an antibody, or a fragment or derivative thereof such as Fab, Fab′, F(ab)2, F(ab′)2, F(ab)3, Fv, single-chain Fv (ScFv), diabodies or VHH, a ligand, a peptide or protein, an aptamer, a polysaccharide, a small organic molecule, a protein tag, or a cation binding group, while the comprises a group which specifically binds said functional moiety, or vice versa.

For example, the interaction between M₁ and Q may rely on cation binding groups (e.g. nitrilotriacetate (NTA, for binding to His tags), iminediacetate or triazacyclononane), protein binding tags or ligand/anti-ligand couples (e.g. antibody/antigen such as biotin/anti-biotin antibody and digoxygenine/anti-digoxigenin antibody, or ligand/receptor).

In a preferred embodiment, M₁ comprises a protein binding tag while Q comprises a protein, a peptide or a fragment thereof which specifically binds said tag, or vice versa.

A multitude of protein tags are well-known by the skilled person (see for example Young et al. Biotechnol. J. 2012, 7, 620-634) and include, for example, biotin (for binding to streptavidin or avidin derivatives), glutathione (for binding to proteins or other substances linked to glutathione-S-transferase), maltose (for binding to proteins or other substances linked to maltose binding protein), lectins (for binding to sugar moieties), c-myc tag, hemaglutinin antigen (HA) tag, thioredoxin tag, FLAG tag, polyArg tag, polyHis tag, Strep-tag, OmpA signal sequence tag, calmodulin-binding peptide, chitin-binding domain, cellulose-binding domain, S-tag, and Softag3, and the like.

For instance, M₁ can comprise a protein tag, e.g. biotin, while Q comprises a group specifically interacting with said tag e.g. streptavidin or avidin, or vice versa.

As another example, Q can comprise an aptamer or an antibody, while M₁ comprises the ligand of said aptamer or said antibody or vice versa.

In a preferred embodiment, M₁ is a biotin while Q is avidin or streptavidin.

2. The Cellular Entity with Chemically Modified Surface According to the Invention

In an additional aspect, the Invention relates to a cellular entity (preferably a cell) having a chemically-modified surface obtained or obtainable by a method according to the Invention as described above, including by the method called “Embodiment 1” or that called “Embodiment 2”.

A preferred cellular entity is a cell as described above, e.g. a mammal cell or a bacterial cell. Regardless the cellular entity with a chemically-modified surface is obtained by the one step or two step method as described above, the resulting cellular entity is characterized in that it comprises tyrosine residues chemically modified with the N-substituted luminol derivative or the N-substituted PhUr derivative used as chemical reagent.

Accordingly, the Invention also relates to a cellular entity (preferably a cell) having at least one chemically-modified tyrosine residue present in a surface component, preferably in a surface protein, which is of formula (C) or (CP):

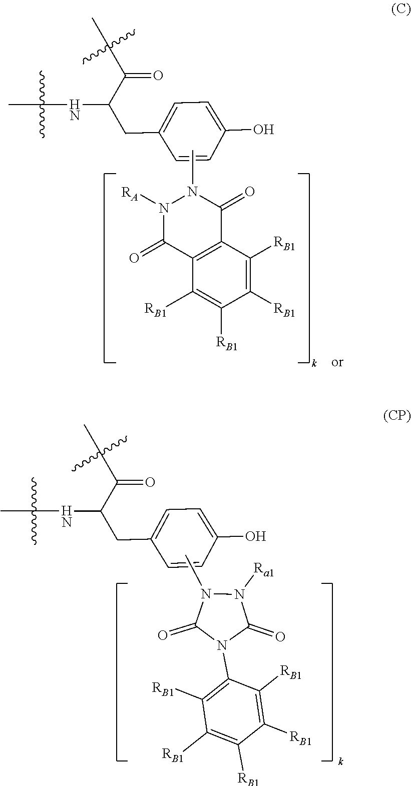

Preferably, the least one chemically-modified tyrosine residue is of formula (C-1) or (C-2) as followed:

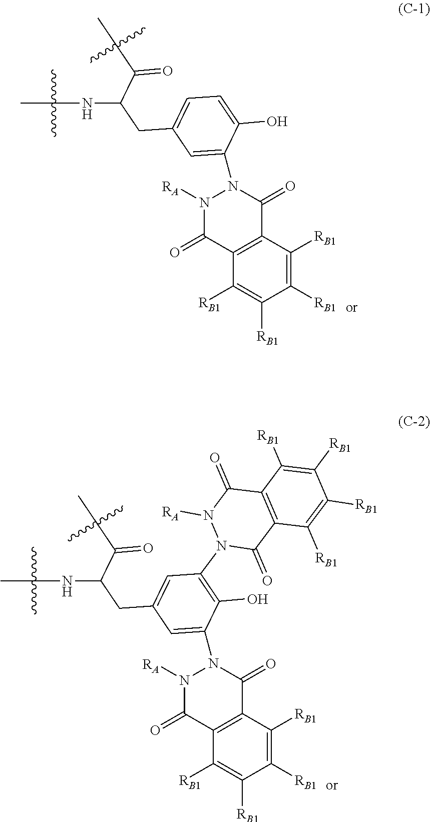

wherein.
k is 1 or 2,
R_{A} is as defined in formula (I) above except that -(Y₁)ₙ-M₁ is replaced by -(Y)ₙ-M. Preferably, R_{A} is a C₁-C₃ alkyl (such as methyl), a phenyl, a benzyl or -(Y)ₙ-M. More preferably, R_{A} is a methyl or a benzyl. Even more preferably, R_{A} is a methyl.
Rₐ₁ is either H or R_{A}, preferably H or a C₁-C₃ alkyl such as methyl,
each R_{B1} is independently selected from a group of formula -(Y)ₙ-M, a hydrogen or a substituent selected from the group consisting of a halogen, C₁-C₆ alkyl, C₆-C₁₄ aryl, C₃-C₆ cycloalkyl, C₁-C₆ alkoxy, C₁-C₆ alkylamino, C₂-C₆ heterocycle, C₁-C₆ alkanoyl, C₁-C₆ carboxy esters, C₁-C₆ acylamino, —COOH, —CONH₂, —NO₂, —SO₃H, —CN, —CF₃, C₁-C₆ hydroxyalkyl, C₁-C₆ haloalkyl, C₁-C₆ thioalkyl, C₁-C₆ alkylthio, C₂-C₁₀ alkoxyalkyl, and C₂-C₆ alkoxycarbonyloxy,
   with the proviso that when the chemically modified tyrosine is of formula (C), (C-1) or (C-2), at least one group among R_{A} and R_{B1} groups is -(Y)ₙ-M and when the chemically modified tyrosine is of formula (CP), at least one group among Rₐ₁ and R_{B1} groups is -(Y₁)ₙ-M₁.
n is 0 or 1,
Y is a spacer, and
M is a functional moiety.

In some embodiments, the chemically modified tyrosine of formula (C) or (CP) is such that R_{A} or Rₐ₁ respectively is -(Y)ₙ-M. Preferably, none of R_{B1} groups is -(Y)ₙ-M. In particular all R_{B1} groups are H.

In preferred embodiments, the chemically modified tyrosine of formula (C) or (CP) is such that one or two (preferably one) R_{B1} is a group of formula -(Y)ₙ-M. Preferably, R_{A} or Rₐ₁ is not -(Y)ₙ-M.

In some additional embodiments, one R_{B}1 is -(Y)ₙ-M and the remaining R_{B}1 are H.

Preferably the chemically-modified tyrosine is of formula (C), in particular of formula (C-3) to (C-6) as follows:

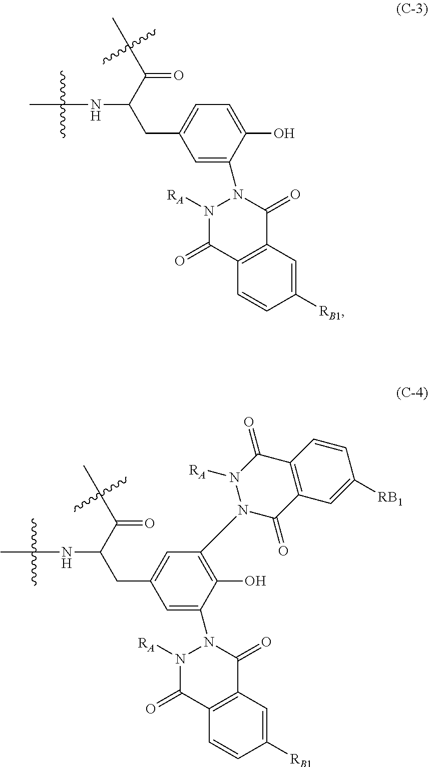

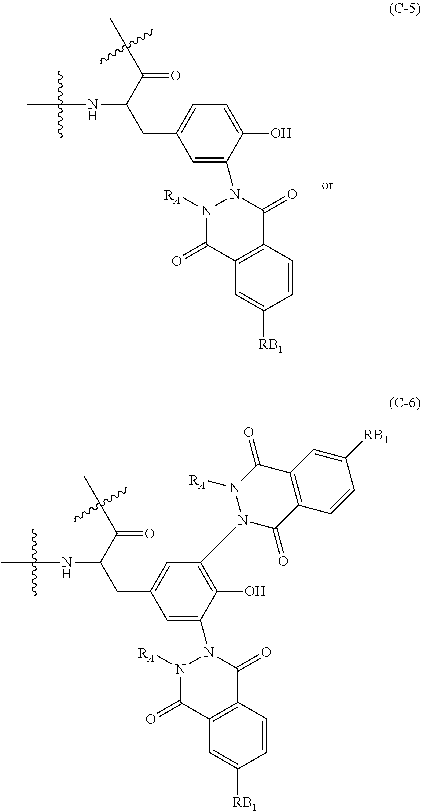

R_{B1} being as defined above, preferably -(Y)ₙ-M and R_{A} being as defined above, preferably a C₁-C₃ alkyl, a phenyl, or a benzyl, more preferably, R_{A} is a methyl.
In the above formula (C) and (C-1) to (C-5) as well as in (CP), -(Y)ₙ-M is such that:
n is 0,
Y is a spacer and
M is a functional moiety.

Typically M can be any type of functional moiety, including those provided for M₁ and M₂ hereabove. M is typically selected depending on the biological effect which is sought by chemically modifying the cell surface.

M can be a reactive group selected so as to enable a subsequent step of coupling, such as a click-chemistry reactive group. M may be also a labelling moiety such as a radionuclide or a fluorophore such as xanthene fluorophores (e.g. fluorescein, rhodamine and derivatives thereof), cyanine fluorophores, BODIPY and the like.

Accordingly, M may comprise or consist of a moiety selected from a chemical reactive group enabling biorthogonal reaction such as a click-chemistry reactive group, a targeting agent, a steric shielding agent, a labelling agent, an oligonucleotide, or a drug, a ligand, a polypeptide, a peptide, a hormone and the like.

For instance, the drug can be an anti-tumoral drug such as a VEGFR inhibitor, an EGFR TK inhibitor, a PIK-1 modulator, a Bcl-2 inhibitor, an HDAC inhibitor, a PARP inhibitor, a Cdk inhibitor, a PI3 kinase inhibitors, a JAK/STAT inhibitor, an immune checkpoint-inhibitor, a focal a Map kinase kinase (mek) inhibitor, Topoisomerase inhibitors, alkylating agents, anti-microtubule agents, and the like. For illustration, the drug can be an anti-PD1 antibody, anticancer small drug such as tamoxifen, imatinib, carboplatin, cisplatin, ifosfamide, cyclophosphamide, methotrexate, fludarabine, 5-fluorouracile, vinblastine, doxorubicin, topotecan, daunorubicin, idarubicin, Monomethyl auristatin E (MMAE) and the like.

“M” may be also a (nano)-particle, including a magnetic (nano-) particle, a quantum dot and liposomes, including liposomes encapsulating drugs or imaging agents. For instance, M1 may be an iron, stain, silicium, gold or carbon (nano)-particle or a phospholipid-based liposome.

In other embodiments, “M” comprises, or consists of, a steric shielding agent, e.g. an agent able to mask certain epitopes present on the cell surface. For instance, “M” may be a polyethylene glycol (PEG), pHPMA or a polysaccharide. “M” may be also an oligonucleotide such as messenger RNA (mRNa) or antisense oligonucleotides such as small interferent RNA (siRNA), shRNA, snoRNA and meroduplex (mdRNA) but also a nucleic ligand such as an aptamer

For instance, “M” may be a ligand which specifically binds to a membrane biological entity (e.g. a membrane receptor). Said ligand may be of any type e.g. a peptide a protein, a saccharide, an oligosaccharide or a small chemical entity. For instance, M may be a mono- or a polysaccharide, a hormone, including a steroid hormone, a peptide, an aptamer, a polypeptide comprising an antigen binding-domain in particular an antibody including heavy-chain antibody, and fragments thereof such as Fab, Fab′, and VHH (also called nanobody), a ScFv, a spiegelmer, a peptide aptamer, biotin, (strept)avidin, cation binding groups, protein tags (e.g. c-myc tag, hemaglutinin antigen (HA) tag, thioredoxin tag, FLAG tag, polyArg tag, polyHis tag, Strep-tag) and the like.

In some embodiments, M is selected from the group consisting of:
a click chemistry reactive group, preferably N₃, tetrazine and cyclooctyne
a polypeptide comprising an antigen binding-domain, preferably a diabody or a nanobody
a cytotoxic or antitumoral drug preferably doxorubicin and monomethylauristatin E
a labelling agent preferably selected from:
   complexes of radionuclides such as 67Cu in DOTA
   fluorophore such as fluorescein and derivatives thereof such as FITC, or Cyanines 3/5/7 (Cy3, Cy5, Cy7)
a shielding or masking agent such as PEG
carbohydrate antigens and oligo- or polysaccharide ligands such as oligo-mannosides or sialosides.
protein tags such as biotin/strep(avidin)

Y may be any chemical chain (e.g. hydrocarbon chain) which can comprise heteroatoms as well as cyclic moieties such as cycloalkyl, cycloalkenyl, aromatic groups, or heterocyclic moieties such as heterocycloalkyl or heteroaryl. Y may comprise up to 2000 carbon atoms, preferably up to 1000 or 500 carbon atoms. The length and the chemical nature of the spacer may be optimized depending on the functional moiety “M” which is intended to be coupled on the cell surface and the biological effect which is sought.

Indeed, further to its linking function, Y1 may be used to refine the properties of the functional moiety “M”. For instance, Y may decrease the steric hindrance of M with respect to the cell surface, or improve the accessibility and the binding of M with a biological entity of interest.

In some embodiments, Y is a chemical chain group comprising from 2 to 2000 carbon atoms, preferably from 2 to 1000 carbon atoms, from 2 to 600 carbon atoms, e.g. from 2 to 200 carbon atoms, 2 to 80 carbon atoms, from 2 to 60 carbon atoms from 2 to 40 carbon atoms or from 2 to 20 atom carbons.

Typically, Y is selected from the group consisting of polymers including homopolymers, copolymers and block polymers, peptides, oligosaccharides, and saturated or unsaturated hydrocarbon chains optionally interrupted by one or several heteroatomic groups (e.g. S, O, Se, P, —C(O)—, —NHC(O)—, —OC(O)—, —N(R)— with R being H or C₁-C₃ alkyl), and/or by one or several cyclic or heterocyclic moieties, and/or optionally having an heteroatomic group (such as S, O, —C(O)—, —NHC(O)—, —OC(O)—, —N(R)— with R being H or C₁-C₃ alkyl, —O—N(R)— with R being H or C₁-C₃ alkyl, or —N(C₁-C₃ alkoxy)-) at least one of its extremity, and/or optionally being substituted by one or several substituents (e.g. hydroxyl, halogens, C₁-C₃ alkoxy, —CN, —CF₃, or C₁-C₃ alkyl), and combinations thereof.

As used herein, “combinations” means that the spacer group Y may comprise several hydrocarbon chains, oligomer chains or polymeric chains (e.g. 2, 3, 4, 5 or 6) linked (or connected) by any appropriate group, such as —O—, —S—, —N(R)— with R being H or C₁-C₃ alkyl, —C(O)—, —NHC(O)—, —OC(O)—, —C(O)—O—C(O)—, —NH—CO—NH—, —O—CO—NH—, NH—(CS)—NH—, NH—CS—, phosphodiester or phosphorothioate groups as well as cyclic or heterocyclic groups.

Typically, the group(s) (also called connectors) used to link the several hydrocarbon chains, oligomer chains or polymeric chains together result from the reactions used to connect these different chains together. For instance, the connector may be —NHC(O)— in case of amide coupling reaction, “N” in case of reductive amination or a triazole derivative in case of click chemistry involving the reaction of an azido with an alkyne group.

In some embodiments, Y may be selected from the group consisting of polyethers such as polyethylene glycol (PEG) and polypropylene glycol, polyvinyl alcohol (PVA), polyesters such as polylactate, polyacrylate, polymethacrylate, polysilicone, polyamide such as polycaprolactone and poly(N-(2-hydroxypropyl)methacrylamide) (pHPMA), poly(D,L-lactic-co-glycolic acid) (PLGA), polymers of alkyl diamines, unsaturated or saturated, branched or unbranched, hydrocarbon chains optionally having an heteroatom such as 0, NH and S on at least one end, and combinations thereof.

As used herein, alkyl diamine refers to NH₂—(CH₂)ᵣ—NH₂ with r is an integer from 2 to 20, for instance from 2 to 10 such as 2, 3, 4, and 5. A polymer of alkyl diamines (also known as polyamines) refers to a compound of formula NH₂—[(CH₂)ᵣ—NH]ₜ—H with r being as defined above and t is an integer of at least 2, for example of at least 3, 4, 5, 10 or more.

For instance, Y can comprise at least one polyethylene glycol moiety comprising from 2 to 40 monomers, e.g. from 2 to 10 or 2 to 6 monomers. For illustration only, Y may comprise from 2 to 10 triethyleneglycol blocks linked together by linkers. As another example, Y may be a C₁₂ hydrophilic triethylene glycol ethylamine derivative. Alternatively, Y1 may be a saturated or unsaturated C₂-C₄₀ hydrocarbon chain, in particular a C₁₀-C₂₀ alkyl chain or a C₂-C₁₀ alkyl chain such as a C₆ alkyl chain. The alkyl chain may have a group such as NH, S or O on at least one end. For instance, Y may be putrescine.

In a particular embodiment, Y is selected from the group consisting of linear or branched C₂-C₄₀ preferably C₂-C₂₀ alkylene chains, polyethylene glycol, polypropylene glycol, pHPMA, PLGA, polymers of diamino alkyl and combinations thereof. Preferably said polyethylene glycol, polypropylene glycol, PLGA, pHPMA and polymer of alkyl diamines comprise from 2 to 80 monomers, preferably from 2 to 10, 10 to 20 or from 20 to 40 monomers.

In some embodiments, Y is a hydrocarbon chain (for instance an alkyl chain) having from 2 to 200 carbon atoms, 2 to 100 carbon atoms, 2 to 80 carbon atoms, from 2 to 60 carbon atoms, from 2 to 40 carbon atoms from 2 to 30 carbon atoms, or from 2 to 20 carbon atoms, optionally interrupted by:
one or more heteroatomic groups chosen from —O—, —S—, —C(O)—, —NHC(O)—, —OC(O)—, —C(O)—O—C(O)—, —N(R)— with R being H or a C₁-C₃ alkyl, —NH—CO—NH—, —O—CO—NH—, NH—(CS)—NH—, and —NH—CS—; and/or
C₅-C₂₀ carbocyclic moieties such as cycloalkyl, cycloalkenyl, or aromatic groups; and/or
one or more heterocyclic moieties having 5 to 20 ring atoms, such as heterocycloalkyls or heteroaryls having 5 to 20 ring atoms;
and optionally having at least one of its extremities, an heteroatomic group chosen from —O—, —S—, —N(R)— with R being H or C₁-C₃ alkyl, —O—N(R)— with R being H or C₁-C₃ alkyl, —N(C₁-C₃ alkoxy), —C(O)—, —NHC(O)—, —OC(O)—, —C(O)—O—C(O)—, —NH—, —NH—CO—NH—, —O—CO—NH—, NH—(CS)—NH—, NH—CS—.

In some embodiments, Y is a hydrocarbon chain (for instance an alkyl chain) having from 2 to 200 carbon atoms, 2 to 100 carbon atoms, 2 to 80 carbon atoms, from 2 to 60 carbon atoms, from 2 to 40 carbon atoms from 2 to 30 carbon atoms, or from 2 to 20 carbon atoms, optionally interrupted by:
one or more heteroatomic groups chosen from —O—, —S—, —N(R)— with R being H or a C₁-C₃ alkyl, —C(O)—, —NHC(O)—, and —OC(O)—; and/or
one or more heterocyclic moieties having 5 to 20 ring atoms, such as heterocycloalkyls or heteroaryls having 5 to 20 ring atoms;
and optionally having an heteroatomic group chosen from —O—, —S—, —N(R)— with R being H or C₁-C₃ alkyl, —O—N(R)— with R being H or C₁-C₃ alkyl, —N(C₁-C₃ alkoxy)-, —C(O)—, —NHC(O)—, and —OC(O)— at least one of its extremities.
   In some embodiments, Y is a spacer of formula (IV):

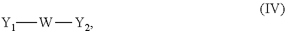

wherein
Y₁ and Y₂ are as defined above. Preferably, each of Y₁ and Y₂ is independently selected from the group consisting of polymers including homopolymers, copolymers and block polymers, peptides, oligosaccharides, saturated or unsaturated, branched or linear hydrocarbon chains, optionally interrupted by one or several heteroatoms (e.g. O, N, S) and/or by a group chosen from —C(O)—, —C(═O)—NH, —C(═O)—O, —C(═O)—O—C(═O)—, O—(C═O)—, NH—C(═O)—, NH—C(═O)—NH, —O—C(═O)—O—, —NH(C═S)—, or —(C═S)—NH—, optionally having at least one of its extremities an heteroatomic group (such as —O—, —S—, —N(R)— with R being H or C₁-C₃ alkyl, —O—N(R)— with R being H or C₁-C₃ alkyl, —N(C₁-C₃ alkoxy)-, —C(O)—, —NHC(O)—, and —OC(O)—), and optionally substituted by one or several substituents, and combinations thereof; and
W is a heterocyclic moiety having 5 to 20 ring atoms, such as heterocycloalkyls or heteroaryls having 5 to 20 ring atoms.

In a particular embodiment, each of Y₁ and Y₂ is selected from the group consisting of polyethylene glycol, polypropylene glycol, pHPMA, PLGA, polymers of diamino alkyl, linear or branched C₂-C₂₀ alkyl chains (optionally interrupted by one or several heteroatoms (e.g. O, N, S) and/or by a group chosen from —C(O)—, —C(═O)—NH, —C(═O)—O, —C(═O)—O—C(═O)—, O—(C═O)—, NH—C(═O)—, NH—C(═O)—NH, —O—C(═O)—O—, —NH(C═S)—, or —(C═S)—NH—, optionally having an heteroatomic group at least one of its extremities (such as —O—, —S—, —N(R)— with R being H or C₁-C₃ alkyl, —O—N(R)— with R being H or C₁-C₃ alkyl, —N(C₁-C₃ alkoxy)-, —C(O)—, —NHC(O)—, and —OC(O)—), and combinations thereof.

In a particular embodiment, Y is formed when carrying the method according to embodiment 1 in which the click reaction is a strain promoted alkyne-azide cycloaddition (SPAAC). Alternative, the synthesis route of Y comprises such an alkyne-azide cycloaddition.

Accordingly, W is a T moiety, namely a triazolyl or a triazolyl fused to another cycle e.g. a dibenzoazepinyl. W is preferably selected in the group of heterocyclic moieties (i), (ii), (iii), (iv), (v), (vi), (vii) and (viii) as described above for “T”.

In some embodiments, Y has at one of its extremities (typically, the extremity linked to M) a heteroatomic group chosen from —O—N(R)— with R being H or C₁-C₃ alkyl, and —N(C₁-C₃ alkoxy), preferably chosen from —O—N(Me)-, —O—NH— or —N(OMe)-.

In some embodiments, Y can also comprise a cleavable group able to release M. The release can be triggered enzymatically or by means of a specific chemical reagent. For instance Y can comprise a trans-cyclooctene (TCO) moiety on which M is coupled e.g. through —OCO—NH— linker. Such a group can release M through a click reaction with a tetrazine molecule in vivo (e.g. by the so-called “click and release” reaction described by Robillard's group—See Ji et al., Chem. Soc. Rev. 2019, 48,1077-1094).

For instance, Y may be one of the following formulae:

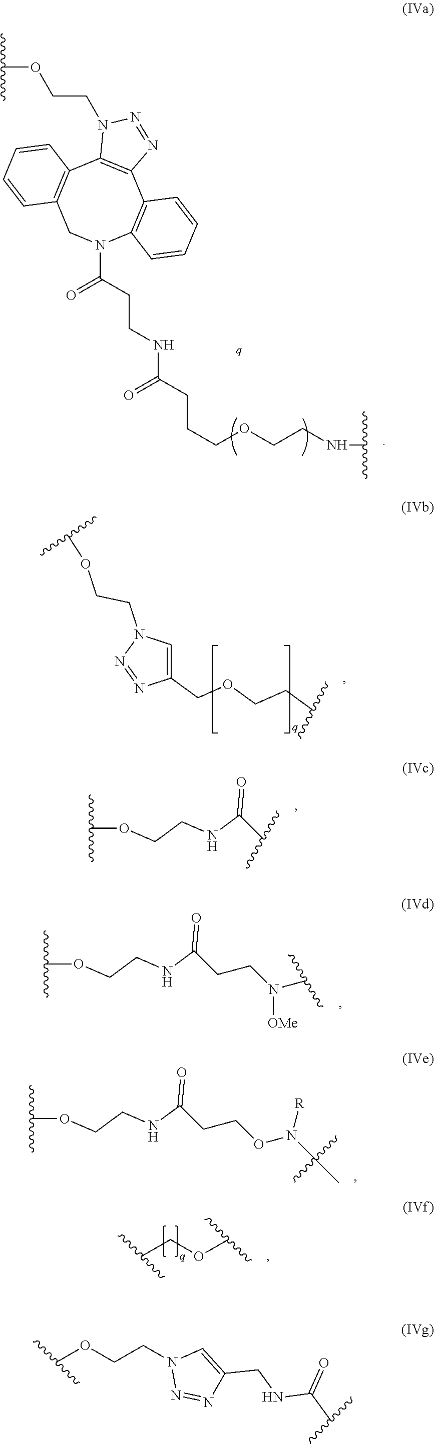

wherein q is an integer from 2 to 10, and R is H or methyl.

### 3. Use of the Chemically-Modified Cellular Entity of the Invention

The method of the invention is a versatile technology enabling to remodel the surface of any kind of cellular entities, including mammal and bacterial cells and extracellular vesicles thereof, by introducing any kind of functional moiety.

The method of the Invention can be thus implemented in any field when one seeks to immobilize a heterogenous entity at the surface of the cell for different purposes, e.g. in research field, in diagnosis field or in therapeutic field.

Depending on the starting cell and the functional moiety immobilized at the cell surface, the chemically modified cell according to the invention can be used in a plurality of applications, including in research, diagnosis and therapy. Some possible uses are provided hereunder, for illustration only, the below list being non exhaustive:

The cell of the invention can be used as research tool, e.g. for studying cell-to-cell or cell-to-matrix interactions, or in tissue-engineering.

The cell of the invention can be used for in vivo imaging, in particular as diagnostic tool e.g. in tomography or MRI

The cell of the Invention can be used as carrier, for in vivo delivery of drugs, e.g. to cross the blood-brain barrier, to increase the circulation time of the drug, and/or to target specific tissues taking advantage of cell tropism and homing. The drug can be present in nanoparticulate cargo attached to the cell surface by the method of the invention, or directly linked to the cell surface.

There is a wide variety of cells that can be used as drug delivery systems such as red blood cells, platelets, dendritic cells, macrophages, T cells and even bacteria.

For instance, the cell can be used in cell therapy such as CAR-T cell therapy for the delivery of immune adjuvant, or in anticancer treatment to deliver drugs to hypoxic tumor tissues, the cell being a anaerobic bacterium or a macrophage.

The method of the invention can be also used to decorate the cell surface with e.g. hyperbranched polyglycerols or PEG to perform immuno-camouflage, which is of interest in the context of organ transplants or blood transfusion.

As another example, decoration of the cell with specific ligands can improve the tropism of the cell and also finds application in organ transplant including bone marrow transplant.

The method of the invention can be also used to remodel the surface of extracellular vesicles.

The method of the invention can be also used to remodel the surface of extracellular vesicles.

The method of the Invention can be thus implemented in any field when one seeks to immobilize a functional moiety on the surface of an extracellular vesicle for different purposes, e.g. in research field, in diagnosis field or in therapeutic field.

Depending on the starting EV and the functional moiety to immobilize, the chemically modified extracellular vesicle (EV) according to the invention can be used in a plurality of applications, including in research, diagnosis and therapy.

Some possible uses are provided hereunder, for illustration only, the below list being non exhaustive:

The chemically modified extracellular vesicle of the invention can be used as a carrier to deliver a large variety of drugs such as small synthetic drugs, therapeutic protein, silencing RNA, and microRNA in vivo, ex vivo or in vitro. Thanks to the method of the invention, said chemically modified extracellular vesicle can have its surface decorated with targeting ligands such as antibodies, oligosaccharides or peptides enabling to specifically target a particular cell type or tissue. In addition or alternatively, said chemically modified extracellular vesicle can be labelled with fluorescent, radioactive or MRI agents to enable in vivo tracking for research or diagnosis purpose. As a further example, said chemically modified extracellular vesicle can be decorated with moieties decreasing its clearance, such as PEG moieties.

The method of the invention can be also used to label extracellular vesicles present in a sample, for instance derived from a body fluid, for diagnosis purpose. The chemically modified vesicles of the invention can thus be used as a biomarker for disease diagnosis or monitoring.

The following examples are given for purposes of illustration and not by way of limitation.

### EXAMPLE SECTION

### Generalities

Most of the chemical reagents and anhydrous solvents were purchased from Sigma Aldrich®, Carbosynth®, Acros Organics®, Alfa Aesar® or TCI Chemical®. All reagents were stored according to the detailed specifications and used without further purification. Reactions requiring anhydrous conditions were performed under positive nitrogen or argon pressure. Usual reaction monitoring was carried out with thin layer chromatography (TLC) on Merck 60 F254 silica gel plates. Revelations were performed under UV light (254 nm) or by dipping in a solution of cerium molybdate, potassium permanganate, sulfuric acid or vanillin and subsequently heated. Purification by silica gel chromatography were carried on Silica 60 M 0.04-0.063 mm. 1H and 13C NMR were recorded on Bruker Avance 300 or Bruker Avance 400 spectrometers. NMR spectra were assigned on the basis of the following 1D and 2D experiments: 1H, 13C, DEPT-135, COSY, HSCQ, HMBC and NOESY. All chemical shifts (6) are shown in ppm on the X-axis using the residual solvent as internal standard. Coupling constants (J) are reported in Hz and peak multiplicities are noted according to the following abbreviations: s=singlet, d=doublet, t=triplet, q=quartet, quin=quintet, m=multiplet, dd=doublet of doublet, dt=doublet of triplet, br=broad signal. Atom numbering used for NMR attribution is different from the numbers used in nomenclature of compounds. High-resolution mass spectrometry (HRMS) was recorded on a Waters Xevo GL-XS Qtof spectrometer coupled with an Acquity H-class LC apparatus. Ionization sources were performed with the available methods (ESI+, ESI−, ASAP+, ASAP−). A tolerance of 5 ppm was applied between calculated and experimental values. Nanodody cyclooctynes CD62L and anti-nanobody antibody were purchased from NanoTag®.

Aqueous buffers were obtained from Sigma Aldrich® or Thermofisher®. Fluorescent cyclooctynes were purchased from Jena Biosciences®.
SP-50 potentiostat was purchased from BioLogic®. Electrosynthesis equipments including ElectraSyn 2.0, electrodes and vials were purchased from IKA®. Chronocoulometric experiments were performed with a three-electrode system connected to SP-50 potentiostat for voltage control. All data were recorded using EC-Lab software. Three-electrode system was typically graphite plate as anode, platinum plate as cathode, and the reference was Ag/AgCl (a thin silver rod submerged with saturated aqueous KCl solution and protected from electrolysis mixture by a porous frit glass). Before each experiment, electrodes used were thoroughly washed with EtOH and distillated water, and working electrode was re-polished on high grit sand paper (<1200 grit) to prevent potential passivation.

### Example 1: Preparation of N-Methyl Luminol Derivative

### Azido Derivative

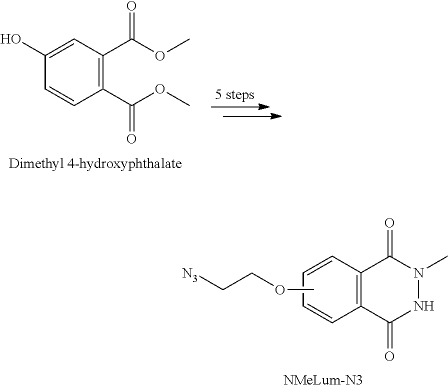

NMeLum-N₃ was prepared in 5 steps from dimethyl 4-hydroxyphthalte as previously reported (S. Depienne, et al., Chem. Sci., 2021, 12, 15374-15381). It is formed as a mixture of 2 regioisomers in 60/40 proportions. ¹H NMR (400.16 MHz, DMSO-d⁶, 298.15 K): δ_{H} 8.15 (d, J=8.8 Hz, 0.4H, H^{Ar}), 7.91 (d, J=8.8 Hz, 0.6H, H^{Ar}), 7.62 (d, J=2.7 Hz, 0.6H, H^{Ar}), 7.46 (dd, J=8.8 Hz, J=2.7 Hz, 0.6H, H^{Ar}), 7.43 (dd, J=8.8 Hz, J=2.7 Hz, 0.4H, H^{Ar}), 7.33 (d, J=2.7 Hz, 0.4H, H^{Ar}), 4.36 (doubled t, 2H, OCH₂CH₂N), 3.72 (doubled t, 2H, OCH₂CH₂N), 3.55+3.53 (doubled s, 3H, NCH₃). ¹³C NMR (100.62 MHz, DMSO-d⁶, 298.15 K): δ_{C} 161.2, 160.8, 157.0, 156.9, 150.2, 149.9, 130.8, 128.7, 126.6, 122.7, 121.8, 121.0, 118.7, 108.3, 106.2, 67.4, 49.4, 37.6, 37.3. HRMS (ASAP⁻): m/z calculated for C₁₁H₁₀N₅O₃ [M−H]⁻ 260.0783 found 260.0784.

### Biotin Derivative

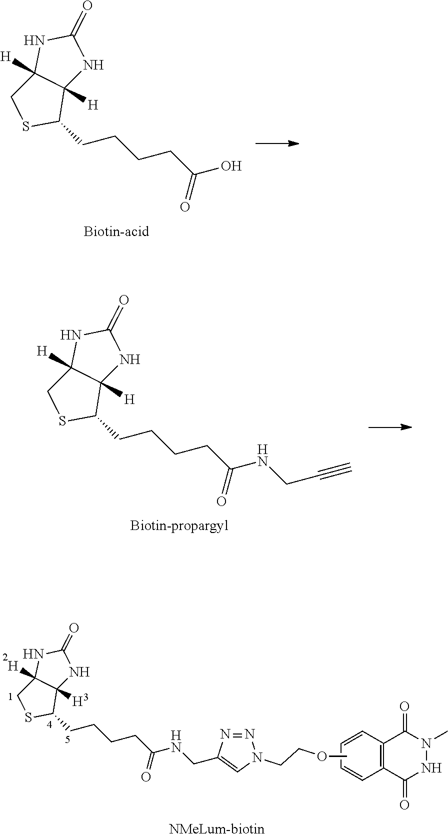

Biotin-propargyl derivative was prepared as described in literature from commercially available Biotin-acid (C.-C. Lin, et al., Org. Lett., 2007, 9, 2131-2134). Then, to a solution of Biotin-propargyl (70 mg, 0.25 mmol, 1.1 equiv.) in dioxane (2 mL) were added luminol-azido derivative (59 mg, 0.22 mmol, 1 equiv.) and sodium ascorbate (54 mg, 0.27 mmol, 1.2 equiv.). An aqueous solution (0.5 mL) of CuSO₄·H₂O (24 mg, 0.13 mmol, 0.6 equiv.) was then added and reaction was heated up to 70° C. and stirred protected from light with aluminium. After 45 min, completion was monitored by TLC (Rf=0.2 in 85:15 DCM/MeOH) and mixture was cooled to room temperature. Chelex Resin® was added (2 spatula) and mixture was left stirred 10 min. Resin was filtered off and washed twice with MeOH. The obtained solution was concentrated under reduced pressure and the residue was purified by silica gel chromatography (90:10 to 85:15 DCM/MeOH) to afford NMeLum-biotin (61 mg, 50%) as a white solid. ¹H NMR (400.16 MHz, DMSO-d⁶, 298.15 K): δ_{H} 8.25 (t, J=5.5 Hz, 1H, NH^{amide}), 8.11 (d, J=8.7 Hz, 0.3H, H^{Ar}), 8.00+7.99 (doubled s, 1H, C═CH^{triazol}), 7.88 (d, J=8.7 Hz, 0.7H, H^{Ar}), 7.56 (d, J=2.5 Hz, 0.7H, H^{Ar}), 7.44-7.35 (2×dd, J=8.7 Hz, J=2.5 Hz, 1H, H^{Ar}), 7.31 (d, J=2.5 Hz, 0.4H, H^{Ar}), 6.38 (br s, 1H, NH^{urea}), 6.33 (br s, 1H, NH^{urea}), 4.79 (m, 2H, NCH₂CH₂O^{luminol}), 4.58 (m, 2H, NCH₂CH₂O^{luminol}), 4.28 (d, J=5.5 Hz, 2H, NCH₂C^{triazol}, overlapped with m, 1H, H²), 4.11 (m, 1H, H³), 3.54+3.52 (doubled s, 3H, NCH₃), 3.08 (m, 1H, H⁴), 2.80 (dd, J=12.3 Hz, J=5.1 Hz, 1H, H^{1a}), 2.57 (dd, J=12.3 Hz, J=2.0 Hz, 1H, H^{1b}), 2.09 (t, J=7.5 Hz, 2H, CH₂CONH^{amide}), 1.65-1.39 (m, 4H, H^{chain} (including H^{5a}+H^{5b})), 1.36-1.21 (m, 2H, H^{chain}); ¹³C NMR (100.62 MHz, DMSO-d⁶, 298.15 K): δ_{C} 172.9, 163.6, 162.0, 161.6, 146.1, 131.7, 129.6, 127.5, 124.2, 123.7, 122.7, 122.0, 109.5, 67.7, 61.9, 60.1, 56.3, 49.68, 35.9, 35.0, 29.1, 28.9, 26.1; HRMS (ES⁻): m/z calculated for C₂₄H₂₉N₈O₅S [M−H]⁻ 541.1982 found 541.1984.

### GalNAc Derivative #

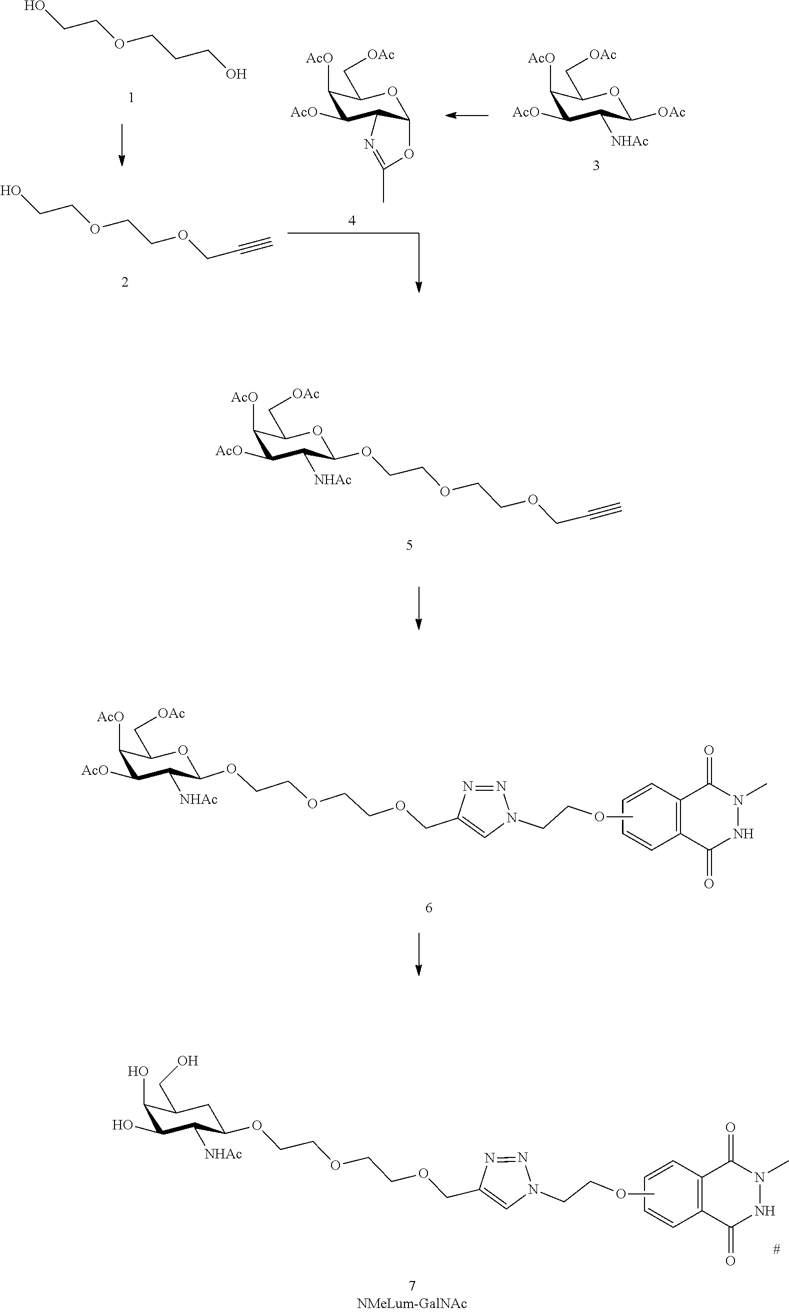

### Compound 2

To a suspension of commercially available tBuOK (793 mg, 7.07 mmol, 1 equiv.) in anhydrous THF (25 mL), commercially available diethylene glycol 1 (1.34 mL, 14.13 mmol, 2 equiv.) was added at 0° C. and under positive nitrogen atmosphere. Mixture was stirred at room temperature during 30 min. 80% propargyl bromide solution in toluene (0.61 mL, 7.07 mmol, 1 equiv.) was then solubilized in 5 mL of anhydrous THF and added dropwise to the mixture. Reaction was stirred at room temperature during 18 h (completion monitored by TLC, Rf=0.3 in pure AcOEt) and mixture was filtered on a celite pad washed with THF. The obtained solution was concentrated under reduced pressure and purified by silica gel chromatography (2:8 CyHex/AcOEt to 100% AcOEt) to afford 2 (735 mg, 72 0%) as a colourless oil. ¹H NMR (300.13 MHz, CDCl₃, 298.15 K): δ_{H} 4.20 (d, J=2.4 Hz, 2H, —CH₂C≡CH), 3.75-3.65 (m, 6H, H^{chain}), 3.60 (m, 2H, H^{chain}) 2.44 (t J=2.4 Hz, 1H, —CH₂C≡CH); HRMS (ES⁺): m/z calculated for C₇H₁₂O₃Na [M+Na]⁺ 167.0684 found 167.0689.

### Compound 4

To a solution of commercially available peracetylated galactosamine 3 (2 g, 5.14 mmol, 1 equiv.) in anhydrous DCM (25 mL, 0.2 M) at 0° C. and under positive nitrogen atmosphere, TMSOTf (3.25 mL, 17.98 mmol, 3 equiv.) was added dropwise. The ice bath was removed and reaction was heated up to 50° C. and stirred during 5 h. Completion was monitored by TLC (Rf=0.3 in 98:2 DCM/MeOH) and reaction was quenched with a saturated solution of NaHCO₃. Aqueous layer was extracted three time with DCM and the combined organic layer was washed once with H₂O and once with brine before being dried over MgSO₄, filtered and concentrated under reduced pressure. The obtained crude compound 4 (1.65 g, orange oily solid) was used in next step without further purification.

### Compound 5

To a solution of freshly prepared crude oxazoline 4 (1.43 g, 4.34 mmol, 1.25 equiv.) in anhydrous DCM (30 mL, 0.1 M), at room temperature and under positive nitrogen atmosphere, mono-O-propargyl chain 3 (500 mg, 3.47 mmol, 1 equiv.) was added. Mixture was cooled to 0° C. and TMSOTf (0.31 mL, 1.74 mmol, 0.5 equiv.) was added. After 24 h stirring at room temperature, completion was monitored by TLC (Rf=0.25 in 95:5 DCM/MeOH) and reaction was quenched with a saturated solution of NaHCO₃. Aqueous layer was extracted once with DCM and the combined organic layer was washed once with H₂O and once with brine before being dried over MgSO₄, filtered and concentrated under reduced pressure. The obtained crude compound was purified by silica gel chromatography (95:5 DCM/MeOH) to afford 5 (670 mg, 42%) as a yellow oil. ¹H NMR (300.13 MHz, CDCl₃, 298.15 K): δ_{H} 6.46 (d, J=9.5 Hz, 1H, NHAc), 5.29 (m, 1H, H⁴), 4.99 (dd, J=11.1 Hz, J=3.4 Hz, 1H, H³), 4.82 (d, J=8.7 Hz, 1H, H¹), 4.34-4.21 (m, 3H, H²+—CH₂C≡CH), 4.14 (m, 2H, H^{6a,b}), 3.92-3.56 (m, 9H, H⁵+8H^{chain}), 2.49 (t, J=2.4 Hz, 1H, —CH₂C≡CH), 2.14 (s, 3H, COCH₃), 2.03 (s, 3H, COCH₃), 1.97 (s, 3H, COCH₃), 1.95 (s, 3H, COCH₃); ¹³C NMR (75.48 MHz, CDCl₃, 298.15 K): δ_{C} 170.6, 170.5, 170.5, 170.4, 102.5, 79.3, 75.4, 72.1, 71.1, 70.6, 70.1, 69.2, 68.2, 66.7, 61.7, 58.3, 50.5, 23.17, 20.8, 20.7, 20.7; HRMS (ES⁺): m/z calculated for C₂₁H₃₁NO₁₁Na [M+Na]⁺ 496.1495 found 496.1797.

### Compound 6

To a solution of prepared 5 (585 mg, 1.24 mmol, 1.1 equiv.) in dioxane (9.5 mL, final conc. 0.1 M) were added prepared azido derivative (293 mg, 1.12 mmol, 1 equiv.) and sodium ascorbate (267 mg, 1.35 mmol, 1.2 equiv.). An aqueous solution (2.5 mL) of CuSO₄·H₂O (120 mg, 0.67 mmol, 0.6 equiv.) was then added and reaction was heated up to 70° C. and stirred protected from light with aluminium. After 45 min, completion was monitored by TLC (Rf=0.35 in 9:1 DCM/MeOH) and mixture was cooled to room temperature. Chelex Resin® was added (4 spatula) and mixture was left stirred 15 min. Resin was filtered off and washed twice with MeOH. The obtained solution was concentrated under reduced pressure and purified by silica gel chromatography (93:7 DCM/MeOH) to afford 6 (680 mg, 83%) as a white solid. ¹H NMR (400.16 MHz, DMSO-d⁶, 298.15 K): δ_{H} 11.57 (br s, 1H, NH), 8.21+8.20 (doubled s, 1H, C═CH^{triazol}), 8.10 (d, J=8.7 Hz, 0.4H, H^{Ar}), 7.87 (d, J=8.7 Hz, 0.6H, H^{Ar}), 7.80 (d, J=9.2 Hz, 1H, NHAc), 7.60 (d, J=2.5 Hz, 0.6H, H^{Ar}), 7.45-7.36 (2×dd, J=8.7 Hz, J=2.5 Hz, 1H, H^{Ar}), 7.30 (d, J=2.5 Hz, 0.4H, H^{Ar}), 5.21 (d, J=3.3 Hz, 1H, H⁴), 4.97 (dd, J=11.2 Hz, J=3.4 Hz, 1H, H³), 4.82 (m, 2H, ArOCH₂CH₂N), 4.60 (m, 2H, ArOCH₂CH₂N), 4.57-4.50 (d+s, J=8.7 Hz, 3H, H¹+OCH₂C^{triazol}), 4.02 (m, 3H, H⁵+H^{6a,b}), 3.87 (m, 1H, H²), 3.77 (m, 1H, H^{chain}), 3.61-3.45 (m, 10H, 7H^{chain}+NCH₃), 2.09 (s, 3H, COCH₃), 1.98 (s, 3H, COCH₃), 1.89 (s, 3H, COCH₃), 1.75 (s, 3H, COCH₃); ¹³C NMR (100.62 MHz, DMSO-d⁶, 298.15 K): δ_{C} 169.9, 169.8, 169.6, 169.3, 161.3, 160.7, 144.0, 130.7, 128.6, 126.6, 124.4, 122.8, 121.8, 121.1, 108.6, 100.9, 70.5, 69.9, 69.7, 69.4, 68.9, 68.2, 66.8, 66.7, 63.4, 61.4, 49.4, 48.8, 22.7, 20.5, 20.4, 20.4; HRMS (ES⁺): m/z calculated for C₃₂H₄₃N₆O₁₄ [M+H]⁺ 735.2837 found 735.2832.

### Compound 7 (NMeLum-GalNAc)

To a solution of prepared 6 (200 mg, 0.27 mmol, 1 equiv.) in anhydrous MeOH (3 mL, 0.1 M) was added a 1 M solution of MeONa in anhydrous MeOH (0.4 mL, 0.4 mmol, 1.5 equiv.) and reaction was left stirred at room temperature under nitrogen atmosphere. After 3 h, completion was monitored by TLC (Rf=0-0.05 in 85:15 DCM/MeOH) and Dowex-50 acidic Resin® (beforehand reactivated with conc. HCl and washed with water and MeOH) was added (4 spatula). Mixture was left stirred 15 min and resin was filtered off and washed twice with MeOH. The obtained solution was concentrated under reduced pressure and lyophilized to afford pure 7, NMeLumGalNAc (100 mg, 61%) as a white solid that did not require further purification. ¹H NMR (400.16 MHz, DMSO-d⁶, 298.15 K): δ_{H} 8.21+8.20 (doubled s, 1H, C═CH^{triazol}), 8.11 (d, J=8.7 Hz, 0.4H, H^{Ar}), 7.88 (d, J=8.7 Hz, 0.6H, H^{Ar}), 7.61 (d, J=2.5 Hz, 0.6H, H^{Ar}), 7.57 (d, J=9.0 Hz, 1H, NHAc), 7.45-7.37 (2×dd, J=8.7 Hz, J=2.5 Hz, 1H, H^{Ar}), 7.31 (d, J=2.5 Hz, 0.4H, H^{Ar}), 4.82 (m, 2H, ArOCH₂CH₂N), 4.61 (m, 2H, ArOCH₂CH₂N), 4.52 (s, 2H, OCH₂C^{triazol}), 4.29 (d, J=8.5 Hz, 1H, H¹), 3.78 (m, 2H, 2H^{chain}), 3.70 (m, 1H, H²), 3.64 (m, 1H, H⁴), 3.58-3.48 (m, 11H, NCH₃+H^{6a,b}+6H^{chain}). 3.42 (dd, J=10.6 Hz, J=3.3 Hz, 1H, H³), 3.30 (t, J=6.1 Hz, 1H, H⁵), 1.77 (s, 3H, NHAc); ¹³C NMR (100.62 MHz, DMSO-d⁶, 298.15 K): δ_{C} 169.5, 161.1, 160.8, 144.0, 130.7, 128.7, 126.6, 124.4, 122.7, 121.8, 121.1, 108.5, 101.3, 75.3, 71.6, 69.7, 69.6, 68.9, 67.5, 66.7, 63.5, 60.5, 52.0, 48.8, 48.6, 22.9; HRMS (ES⁺): m/z calculated for C₂₆H₃₇N₆O₁₁ [M+H]⁺ 609.2520 found 609.2523.

### Example 2: Electrochemical Behaviour

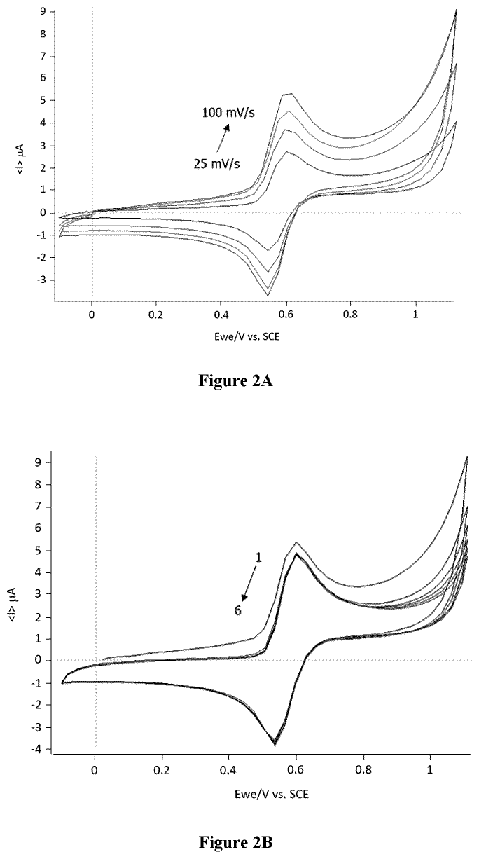

Electrochemical behaviour of luminol derivatives was measured by cyclic voltammetry and multicyclic voltammetry at 1 mM in 1:1 MeCN/PB 100 mM pH 7.4 and recorded at a 2 mm disc glassy carbon electrode cathode, with a platinum cathode and saturated calomel electrode or silver chloride (saturated KCl) electrode as reference. For cyclic voltammetry, scan rates of 25, 50, 75, 100 and 250 mV/s were recorded between −0.1 and 1.1 V (example in FIG. 2A). For multicyclic voltammetry, 6 cycles were recorded at 100 mV/s between −0.1 and 1.1 V (example in FIG. 2B). Between each experiment, GCE electrode was repolished on high grit sand paper to prevent potential passivation. Cyclic voltammetry of compounds in pure aqueous buffers using silver chloride (saturated KCl) reference electrode were also performed to obtain their accurate oxidation potential in electro-bioconjugation conditions.

### Example 3: Electrobioconjugation in Bacteria

### Protocol of Bioconjugation Followed by SPAAC Reaction

TOP10 Escherichia Coli or Staphylococcus Epidermidis strains were precultured in lysogeny broth (LB) until optical density OD₆₀₀=0.5 approx. Then, 250 μL of preculture media were diluted in 25 mL LB and incubated at 180 rpm and 20° C. during 16 h (OD₆₀₀=0.4-0.6 approx.). Bacteria were centrifuged at 8000 rpm during 5 min, supernatant was withdrawn followed by resuspension in 25 mL PBS pH 7.4. Next, to 2.5 mL of the bacterial strain (E. Coli or Staph. Epidermidis) solution in PBS pH 7.4 were added 2.5 mL of 2 mM azido luminol derivative (final conc. 1 mM) solution in PBS pH 7.4. The 5 mL scale Electrasyn setup was assembled and 750 mV vs Ag/AgCl were applied during the studied time at room temperature at 500 rpm. After modification, the excess of unreacted luminol anchor was removed by performing three times the following sequence: i) centrifugation (12000 rpm during 2 min), ii) supernatant withdrawal, iii) bacteria resuspension in 1 mL PBS. At the end of 3rd sequence, bacteria were resuspended in 190 μL PBS pH 7.4 and 10 μL of a 2 mM DBCO-PEG4-CR110 (obtained from Jena Bioscience®) solution (final conc. 0.1 mM) in DMSO were added. The sample was incubated at 23° C. in the dark during 1 h under moderate orbital shaking. Then, the excess of unreacted cyclooctyne was removed by performing four times the previous centrifugation/removal/washings (resuspension included 0.5% DMSO for the two first sequences). At the end of 4th sequence, bacteria were resuspended in the appropriate volumes/solutions for characterizations.

### Characterizations

### Bacterial Viability

Materials and methods: Directly after the electro-conjugation step, 300 μL of the samples (OD₆₀₀=0.6) were 2 times centrifuged (12000 rpm during 2 min)/supernatant withdrawn/resuspended in 1 mL LB, and with final resuspension in 300 μL LB. 80 μL of the washed samples were seeded (each condition in triplicate) in a 24-well plate, diluted to 1 mL LB (OD₆₀₀=0.05), and incubated at 180 rpm at 20° C. OD₆₀₀ were regularly measured on an Infinite M1000 Microplate reader from TECAN using Magellan Software.
Results: Bacteria viability was evaluated by their ability to grow in culture conditions. The longest electro-conjugation conditions (1 h) was performed in triplicate and evaluated. A sample of bacteria left at room temperature during 1 h was also investigated. Each of these conditions were cultured in triplicate and OD₆₀₀ were measured every hour. As a result, electro-conjugated bacteria are still able to grow at the same average rate as unmodified conditions (see FIG. 3 for E. Coli example) for both strains.

### Fluorescence Microscopy and Flow Cytometry

Materials and methods: Electrobioconjugation efficiency was visualized by fluorescence microscopy (FITC excitation conditions) using Nikon clipse NI-E microscope (data treated with NIS software) and quantified by flow cytometry using CYTOFLEX cytometer from Beckman Coulter®—Life Sciences (data treated with FlowJo software).
Results: Fluorescence microscopy evidenced a visual strong labeling of the bacterial membranes for all E. coli and S. epidermidis samples subjected to two-step sequence 1) electro-conjugation with azido derivative and 2) incubation with the fluorescent cyclooctyne (see FIG. 4 for Staph. Epidermidis example). As a stark contrast, fluorescence of bacteria only incubated with the fluorescent cyclooctyne was barely detectable. This control highlights very poor unspecific adsorption or alkyne side-reaction with cell surface thiols during second step and supports the efficient covalent azido labelling during electro-conjugation. On the other hand, mean fluorescence quantified by flow cytometry outlined a clear and relevant time-dependent increase of fluorescence intensity—observed for both bacterial strains—after electro-bioconjugation for 15-, 30- and 60 min (see FIG. 5). The levels of labeling observed in less than 1 h of electro-conjugation showed the efficiency of the methodology to label g(+) and g(−) bacteria harboring highly different cell membrane structures.

### Proteins Membrane Extraction and SDS-PAGE

Materials and methods: 800 μL of each sample were split in 2×400 μL. The latter were centrifuged and supernatants removed. First part was resuspended in 50 μL of resuspension buffer (sodium phosphate buffer 50 mM pH 7.4/300 mM NaCl/2 mM MgCl2/DNASE 1000×/Lysozyme 100×/Protease inhibitor cocktail 200×) and 10 μL of Laemmli 6× buffer were added (Total fraction). Second part was resuspended in 500 μL of resuspension buffer and lysed by 3× periodic 5 sec ON/OFF ultrasonication followed by centrifugation at 12000 g during 30 min. Supernatant was taken off and concentrated using 3K MWCO VWR® centrifugal filters until 80 μL final volume, and 10 μL of Laemmli 6× buffer were added (Cytosolic fraction). The remaining centrifugated pellet was resuspended in 50 μL of 8M urea/50 mM NaH2PO4/300 mM NaCl buffer and 10 μL of Laemmli 6× buffer were added (Membrane fraction). For each sample, the 3 fractions were heated at 95° C. during 5 min (10 min for total fraction) and 10 μL (20 μL for cytosolic fractions) were deposed on a 12% acrylamide gel. Proteins were separated at 90 V during 10 min then at 150 V during 1 h 15. Gels were visualized under UV, washed 3×10 min with water and then stained with Coomassie brilliant blue: colouration overnight and 3×30 min decolouration with water.
Results: All bacteria samples were lysed. Cytosolic and membrane proteins were extracted and fractionated, and analyzed by SDS-PAGE on gel as compared to total lysed fraction. Coomassie brilliant blue (CBB) staining showed a rich and diverse population of proteins in the total samples, and in both cytosolic and membrane fractions as well. To our delight, fluorescence detection exclusively evidenced fluorescent-labelled proteins of various molecular weights in both total and membrane fractions, while the cytosolic proteins and peptides were not modified (see FIG. 6 for E. Coli example)

### Example 4: Electrobioconjugation in Mammalian Cell

### Protocol of Electro Bioconjugation

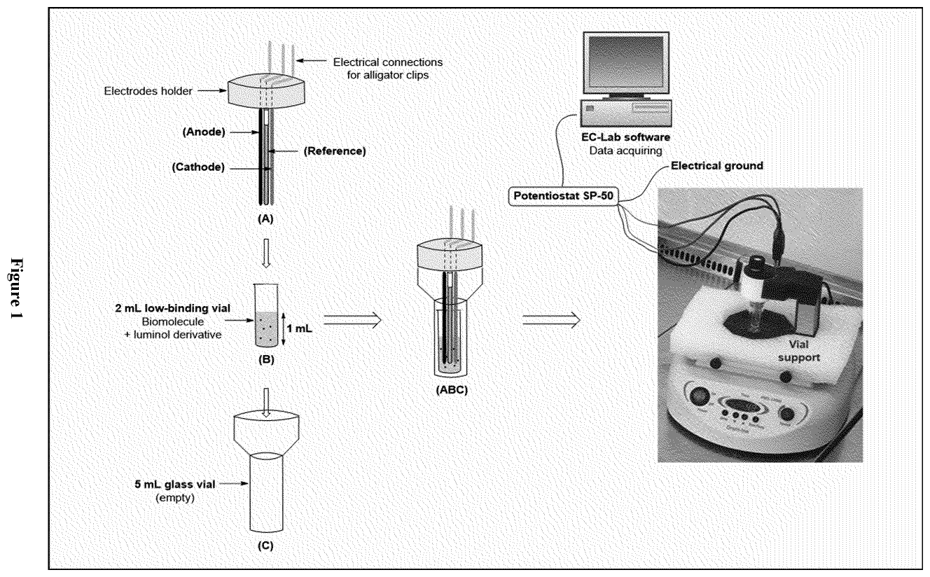

Cells (HEK293 or HeLa) were cultured with 10% FBS serum and 1% penicillin-streptomycin at 37° C. with 5% CO₂. CD2.4 were cultured with RPMI medium, 10% FBS serum, 1% penicillin-streptomycin, 1% Hepes 1M, 1% 2-Mercaptoethanol a 50 mM, 1% MEM NEAA 100× and 1% sodium Pyruvate 100 mM at 37° C. with 5% CO₂. The cells were trypsinized and harvested in PBS pH 7.4 at a concentration of 6.10⁶ cells/mL.
Jurkat cells in suspension were cultured with RPMI medium, 10% FBS serum, 1% penicillin-streptomycin and 4 mM of glutamine solution at 37° C. with 5% CO₂. EXPIf cells in suspension were cultured with BalanCD medium and 4 mM of glutamine solution at 37° C. with 8% CO₂.
In a 2 mL low-binding vial, 500 μL of the cell solution (final conc. 3.10⁶ cells/mL) were added to a solution of NMeLum-N₃ or NMeLum-GalNAc derivative in PBS pH 7.4 (final conc. 0.1 mM or 1 mM—final volume: 1 mL).
The 1 mL scale setup was assembled (see FIG. 1) and 750 mV vs Ag/AgCl were applied during the studied time at room temperature under gentle orbital shaking. After modification, the excess of unreacted N-methyl luminol anchor was removed by performing three times the following sequence: i) centrifugation (2500 rpm during 2 min), ii) supernatant withdrawal, iii) cells resuspension in 1 mL PBS.
At the end of 3rd sequence, cells modified with NMeLum-N₃ were resuspended in 250 μL PBS pH 7.4 and 250 μL of a 0.2 mM DBCO-PEG4-Fluorescein (from Jena Bioscience®) solution in PBS were added (final conc. 0.1 mM) or nanobody cyclooctynes CD62L (from NanoTag®) solution in PBS were added (final conc. 0.01 mM). The sample were incubated at 23° C. in the dark during 1 h under moderate orbital shaking. Then, the excess of unreacted cyclooctyne was removed by performing three times the previous centrifugation/removal/washings with 1 mL PBS. At the end of 3rd sequence modified cells were resuspended in the appropriate volumes for characterizations.
The cells modified with DBCO-CD62L nanobody were then incubated with labelled fluorescein anti-CD62L nanobody antibody at 23° C. in the dark during 1 h under moderate orbital shaking (dilution 1/2500). The excess of unreacted antibody was removed by performing three times the previous centrifugation/removal/washings with 1 mL PBS. At the end of 4th sequence modified cells with DBCO-CD62L nanobody were resuspended in the appropriate volumes for characterizations.
The cells modified with NMeLum-GalNAc derivative were then incubated with labelled fluorescein soybean lectin at 23° C. in the dark during 1 h under moderate orbital shaking (20 μg/mL). The excess of unreacted lectin was removed by performing three times the previous centrifugation/removal/washings with 1 mL PBS. At the end of 4th sequence, modified cells with GalNAc were resuspended in the appropriate volumes for characterizations.

### Protocol of Bioconjugation by Glyco-Engineering Followed by SPAAC Reaction (Comparative)

For glyco-engineering, CD2.4 cells were plated in a 24 well plate at a density of 1.10⁵ cells/mL with media supplemented with Ac4ManNAz (from 50 to 250 μM) and incubated in a humidified atmosphere of 95% air and 5% CO₂ at 37° C. for 72 h. Following this, cells were washed three times with PBS, incubated with DBCO-fluorescein and washed three times with PBS. The cells were then trypsinized, harvested and resuspended in the appropriate volumes for characterizations.

### Protocol of Bioconjugation Followed by Streptavidin-Biotin Complex Formation

Cells (HEK293 or HeLa) were cultured with 10% FBS serum and 1% penicillin-streptomycin at 37° C. with 5% CO₂. The cells were trypsinized and harvested in PBS pH 7.4 at a concentration of 6.10⁶ cells/mL. Then, 500 μL of the cell solution (final conc. 3.10⁶ cells/mL) were added in a 2 mL low-binding vial and centrifuged (2500 rpm during 2 min). Supernatant was withdrawn and 1 mL of a 1 mM solution of luminol-biotin derivative in PBS pH 7.4 were added. The 1 mL scale setup was assembled (see FIG. 1) and 750 mV vs Ag/AgCl were applied during the studied time at room temperature under gentle orbital shaking. After modification, the excess of unreacted luminol anchor was removed by performing three times the following sequence: i) centrifugation (2500 rpm during 2 min), ii) supernatant withdrawal, iii) cells resuspension in 1 mL PBS. At the end of 3rd sequence, cells were resuspended in 25 μL of a 1 g/L solution of Fluorescein-conjugated Streptavidin (obtained from Dako®) in 50 mM Tris/15 mM NaN_{3/1}% BSA pH 7.2. The sample were incubated at 23° C. in the dark during 1 h under moderate orbital shaking. Then, the previous centrifugation/removal/washings sequence was performed three times with 1 mL PBS. At the end of 3rd sequence modified cells were resuspended in the appropriate volumes for characterizations.

### Characterizations

### Cell Viability

### Materials and Methods:

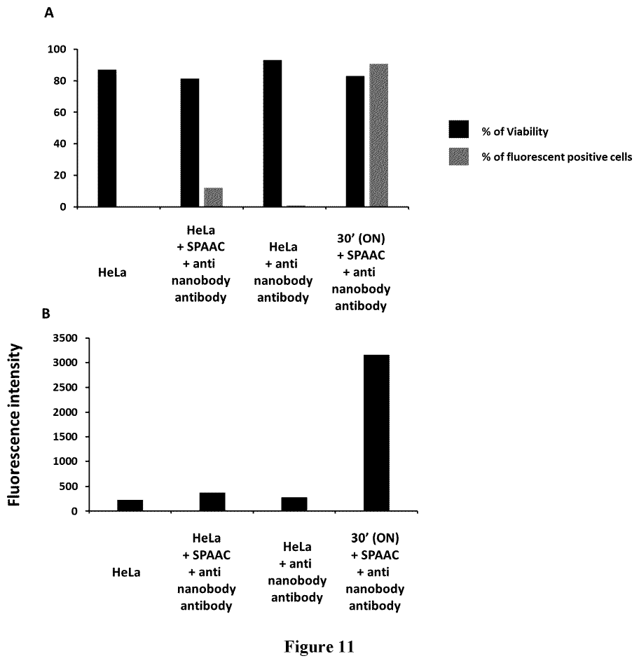

Viability test with trypan blue: Directly after the electro-conjugation step, approx. 5.10⁵ cells in 600 μL PBS were subjected to automatized trypan blue viability test using Vi-CELL XR (from Beckman Coulter, Life Sciences). % of viable cells is calculated from the ratio of positive viable cells and total cells.
Viability test with Zombie Yellow: Directly after the electro-conjugation step, approx. 2.10⁶ electro-conjugated cells were resuspended in 1 mL of freshly prepared Zombie Yellow staining buffer (100 μL Zombie Yellow BV605 from Biolegend® diluted in 1 mL with Brilliant Violet Stain Buffer from BD Biosciences®) and incubated at RT in the dark. Centrifugation/supernatant withdrawal/PBS washing sequence was performed twice and the cells were analyzed by flow cytometry to evaluate proportions of dead cells (Zombie Yellow positive cells).
Viability test with live and dead assay: Directly after the bioconjugation step, approx. 2.10⁶ bioconjugated cells were resuspended in 100 μL of freshly prepared live and dead staining buffer and incubated at 4° C. in the dark. Centrifugation/supernatant withdrawal/PBS washing sequence was performed twice and the cells were analyzed by flow cytometry to evaluate proportions of dead cells (live and dead positive cells).
Viability test via cell culture: Cell viability after modification was evaluated by cell culture. Unmodified, controls and conjugated cells were seeded in DMEM with 10% FBS serum and 1% penicillin-streptomycin in a 24-well culture plate and incubated at 37° C. with 5% CO₂. Growth ability and confluences were evaluated, quantified and compared by microscopy and using Vi-CELL XR after 24 h, 48 h and 72 h.
   Results: For HeLa and HEK cells, viability was evaluated by standard assays (trypan blue, zombie yellow) and by their ability to grow in culture conditions. The impact of the centrifugation/washings sequences was also investigated. For both cell lines, >90-95% average viability was quantified with both trypan blue and zombie yellow assays in all conditions (FIG. 7). Also, fitness of both cell lines was not affected as they fully conserved their ability to divide in cell culture, as quantified after 24 h and 48 h culture.
   For Jurkat, EXPIf, HeLa and DC2.4 cells, viability was evaluated by live and dead standard assay. The impact of the centrifugation/washings sequences was also investigated. For both cell lines, >84% viability was quantified with live and dead assay in all conditions (Table 1 hereunder, FIG. 10A, FIG. 11A).
   For glyco-engineering (comparative), with the concentration of 250 μM the viability is only of 30%. This was also confirmed by Ruben et al. (biomacromolecules, 2019, 20, 2726) where they showed that it is not possible to use more than 100 μM concentration of Ac4ManNAz.

### Flow Cytometry

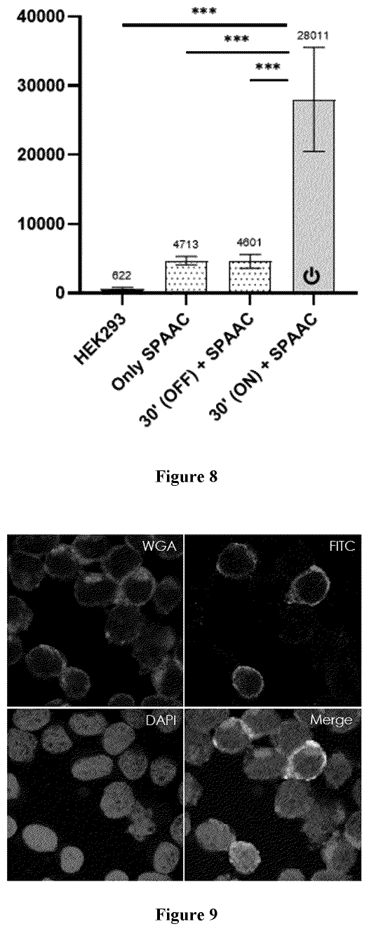

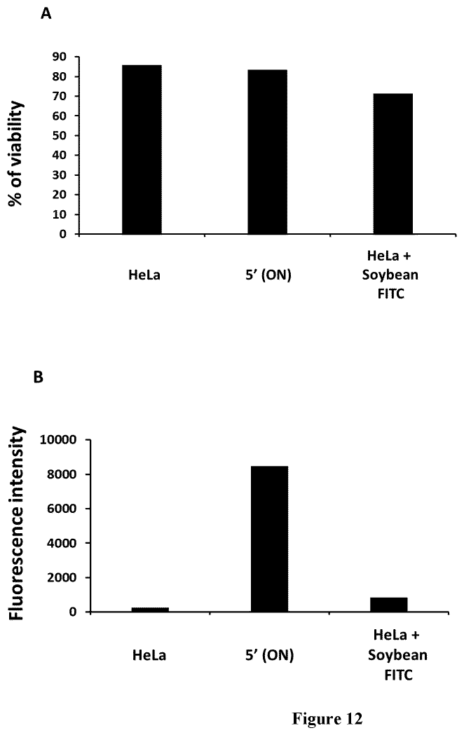

Materials and methods: Samples were analyzed on BD-LSRII Flow Cytometer (BD Bioscience) considering FITC functionalization and detection. All data were processed by FlowJo (V10, FlowJo LLC, Ashland, OR).
Results: Electro-bioconjugation efficiency was evaluated by quantification of mean fluorescence using flow cytometry. A control experiment was also performed where luminol derivative was incubated with the cells for 30 min in first step but without applying the 750 mV potential difference. After the washings, the sample was subjected to FITC tagging with cyclooctyne-FITC or to nanobody tagging with cyclooctyne-CD62L nanobody followed (for cells modified with N-methyl luminol azido derivative), with Streptavidin-FITC (for cells modified with N-methyl luminol biotin derivative) or with FITC-Soybean lectin (for cells modified with NMeLum-GalNAc).
For HeLa, HEK, Jurkat, EXPIf and DC2.4 cell lines, mean fluorescence intensity of this control was virtually identical compared to unmodified cells incubated with FITC probes only. The results clearly indicate that N-methyl luminol derivatives do not tag the cell surface without electro-oxidative activation. However, when the potential difference was effectively applied, a strong signal was satisfyingly detected after 30 min, for all cell lines (HeLa, HEK, Jurkat, EXPIf and DC2.4) and for all N-methyl luminol derivatives (azido, biotin, GalNAc) (see FIG. 8 for HEK293 cell line with NMeLum-N₃, Table 1 hereunder, FIG. 10, FIG. 11 and FIG. 12).
Of note, the mean fluorescence intensity of the electro-bioconjugation is 6 time higher than that obtained with glyco-engineering (FIG. 10), which shows the high efficiency of the electro-bioconjugation method of the invention for cell membrane modification as compared to the methods of the prior art.

| | | | Fluorescence |
|---|---|---|---|
| Cells | Conditions | % of viability | intensity |
| Jurkat | Cells | 99.6 ± 0.1 | 457 ± 32 |
| | 30′ (ON- | 87.2 ± 4.8 | 9174 ± 2791 |
| | electrobioconjugation) + | | |
| | SPAAC | | |
| | Only SPAAC | 99.4 ± 0.1 | 1292 ± 56 |
| EXPI-f | Cells | 98.6 ± 0.2 | 299 ± 17 |
| | 30′ (ON | 84.5 ± 0.5 | 3906 ± 65 |
| | electrobioconjugation) + | | |
| | SPAAC | | |
| | Only SPAAC | 95.8 ± 0.4 | 1099 ± 73 |

Table 1 shows viability (n=3) and mean fluorescence intensity measured by flow cytometry (n=3) for unmodified Jurkat and EXPIf cells, cells incubated only with fluorescent cyclooctyne (Only SPAAC), and effective electro-bioconjugation experiments (final concentration 0.1 mM) followed by incubation with fluorescent cyclooctyne (30′ ON+SPAAC).

### Fluorescence Confocal Microscopy

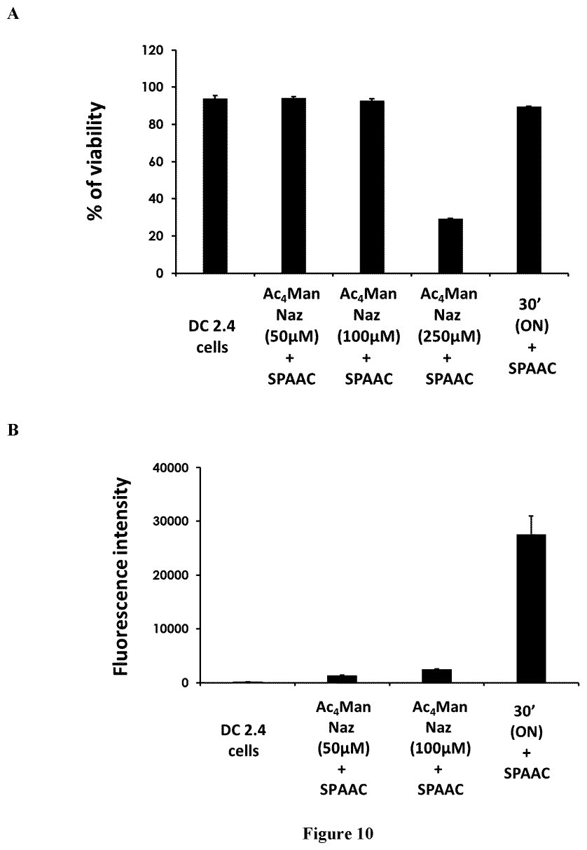

Materials and methods: Membrane mapping was performed by incubating electro-conjugated/FITC-tagged samples with 100 μL of Wheat Germ Agglutinin-AF647 lectin solution (from Invitrogen™, diluted with PBS 1:1000) 30 minutes at 4° C. Two centrifugation/supernatant withdrawal/washings were performed with 250 μL of Perm/Wash Buffer (BD Cytofix/Cytoperm™ Fixation/Permeabilization Solution Kit, Fisher Scientific). The cells were then permeabilized with 100 μL of Fixation/Permeabilization solution (BD Cytofix/Cytoperm™ Fixation/Permeabilization Solution Kit, Fisher Scientific) and incubated 20 minutes at 4° C. Two washings were performed the same way as before and nuclei were stained with 100 μL of DAPI solution (from Sigma Aldrich®, diluted with PBS 1:1000) during 15 minutes at RT. The washings sequence was eventually performed before microscopy. Fluorescence imaging of the cells was performed on a Nikon AIR confocal microscope using a 6041.4 objective. The different channels were recorded as follows: excitation 405 nm: emission recorded from 425 to 475 nm; excitation 488 nm: emission recorded from 500 to 550 nm; and excitation 640 nm, emission recorded from 660 to 740 nm. The gain, offset and the power of lasers were adjusted as needed. Three-dimensional digital images were collected using NIS-Elements confocal software and appropriate fluorescence filters.
Results: The experiment was performed on HEK cells subjected to electrobioconjugation with NMeLum-N₃ followed by SPAAC with FITC-cyclooctyne as described above. Membrane labelling by electro-conjugation was visualized by confocal microscopy. Individual fluorescence imaging of the probes (FITC for electro-bioconjugation, DAPI for nuclei, AF647 for membrane) showed unambiguous overlapping signals for AF647 and FITC when merged, clearly standing for a membrane and not intracellular labelling during electro-conjugation (FIG. 9).

1-19. (canceled)

20. An in vitro method for chemically-modifying the surface of a cellular entity, which comprises incubating said cellular entity with a chemical reagent bearing a N-substituted luminol moiety or a N-substituted phenyl-urazole in conditions conducive for reacting said chemical reagent with a surface component of said cellular entity so as to form a covalent bond and wherein the cellular entity is a cell or a extracellular vesicle.

21. The method of claim 20 which is performed by electrochemistry.

22. The method of claim 20, which comprises the step of incubating the cellular entity with a chemical reagent of formula (I) or (IP): 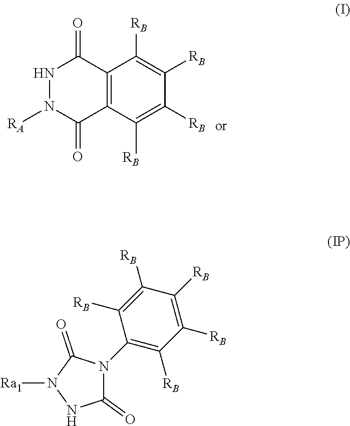
wherein:
R_{A} is a C₁-C₆ alkyl, a C₆-C₁₄ aryl optionally substituted, a (C₆-C₁₄ aryl)-(C₁-C₃ alkyl) optionally substituted or -(Y₁)ₙ-M₁,
Rₐ₁ is H or R_{A},
each R_{B} is independently selected from a group of formula -(Y₁)ₙ-M₁, a hydrogen or a substituent chosen from a halogen, C₁-C₆ alkyl, C₆-C₁₄ aryl, C₃-C₆ cycloalkyl, C₁-C₆ alkoxy, C₁-C₆ alkylamino, C₂-C₆ heterocycle, C₁-C₆ alkanoyl, C₁-C₆ carboxy esters, C₁-C₆ acylamino, —COOH, —CONH₂, —NO₂, —SO₃H, —CN, —CF₃, C₁-C₆ hydroxyalkyl, C₁-C₆ haloalkyl, C₁-C₆ alkylthio, C₁-C₆ thioalkyl, C₂-C₁₀ alkoxyalkyl, and C₂-C₆ alkoxycarbonyloxy,
n is 0 or 1,
Y₁ is a spacer, and
M₁ is a functional moiety;
with proviso that when the chemical reagent is of formula (I), at least one group among R_{A} and R_{B} groups is -(Y₁)ₙ-M₁ and when the chemical reagent is of formula (IP), at least one group among Rₐ₁ and R_{B} groups is -(Y₁)ₙ-M₁,
in the presence of a potential difference enabling the electro-activation of said chemical reagent of formula (I) or (IP) into an oxidized form able to react with a tyrosine residue present in a cell surface component so as to obtain at least one chemically-modified tyrosine residue of formula (B) or (BP) respectively, on the surface of the cellular entity: 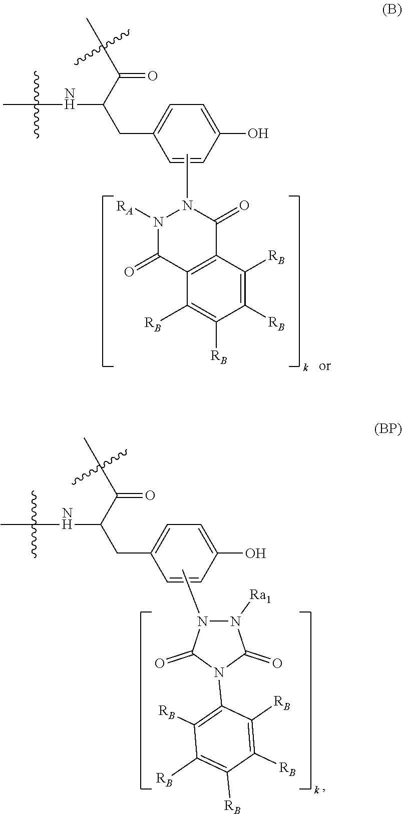
wherein:
R_{A}, Rₐ₁, and R_{B} are as defined in formula (I) and (IP),
k is 1 or 2.

23. The method of claim 22, wherein:
M₁ comprises or consists of a moiety selected from the group consisting of a chemical reactive group enabling biorthogonal reaction, a targeting agent, a steric shielding agent, a labelling agent, an oligonucleotide, a drug, a nanoparticle, and a ligand, and/or
Y₁ is a chemical chain group comprising from 2 to 500 carbon atoms and selected from the group consisting of polymers including homopolymers, copolymers and block polymers, peptides, oligosaccharides, and saturated or unsaturated hydrocarbon chains optionally interrupted by one or several heteroatoms and/or by one or several cyclic or heterocyclic moieties, optionally having an heteroatom, at least one extremity, and optionally substituted by one or several substituents, and combinations thereof.

24. The method of claim 22, wherein the chemical reagent is of formula (I-c): 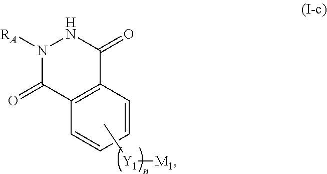
wherein R_{A} is C₁-C₃ alkyl, a phenyl optionally substituted, or a benzyl optionally substituted.

25. The method of claim 21, wherein the method is performed in an electrochemical system with three electrodes comprising a working electrode, a counter-electrode and a reference electrode by applying a constant potential difference between the working electrode and the reference electrode, the potential difference being in a range defined as the oxidation potential of the chemical reagent±200 mV.

26. The method of claim 20, wherein the cellular entity is selected from procaryotes and eucaryotic cells.

27. The method of claim 26, wherein the extracellular entity is selected from:
bacteria,
human cell lines, cells isolated from subjects, erythrocytes, Hematopoietic Stem cells (HSC), Peripheral Blood Mononuclear cells (PBMC), T lymphocytes or dendritic cells (DC),
with proviso that the cell is not a human embryo or is not obtained by a method resulting in destruction of human embryo.

28. A method for decorating the surface of a cellular entity with a functional moiety M₂, which comprises:
(i) a step of chemically modifying the surface of the cellular entity according to the method of claim 22, wherein M₁ is a click chemistry reactive group
(ii) a step of immobilizing M₂ at the surface of the cellular entity by promoting a click reaction with M₁, by incubating the cellular entity obtained in step (i) with a compound of formula (III), 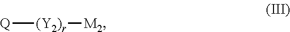
wherein:
Q is a click-chemistry group that is able to react with M₁ through a click chemistry reaction,
r is 0 or 1,
Y₂ is a spacer, and
M₂ is the functional group to be immobilized on the surface of the cellular entity, in conditions conducive to enable the reaction of Q with M₁.

29. The method of claim 28, wherein M₁ is an azido and Q is a strained-alkyne, the click reaction being a strain promoted alkyne-azido cycloaddition (SPAAC).

30. A method for decorating the surface of a cellular entity with a functional moiety M₂, which comprises:
(i) a step of chemically modifying the surface of the cellular entity according to the method of claim 22,
(ii) a step of incubating the cellular entity obtained in step (i) with a compound of formula (III), 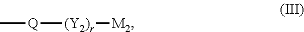
wherein:
r is 0 or 1,
Y₂ is a spacer, and
M₂ is the functional group to be immobilized on the surface of the cellular entity, and
Q and M₁ are selected so as to specifically interact together and form a stable complex in conditions conducive to enable the formation of a complex between Q and M₁.

31. A cellular entity having at least one chemically-modified tyrosine residue present in a cell surface component or a cell surface protein, which is of formula (C) or (CP): 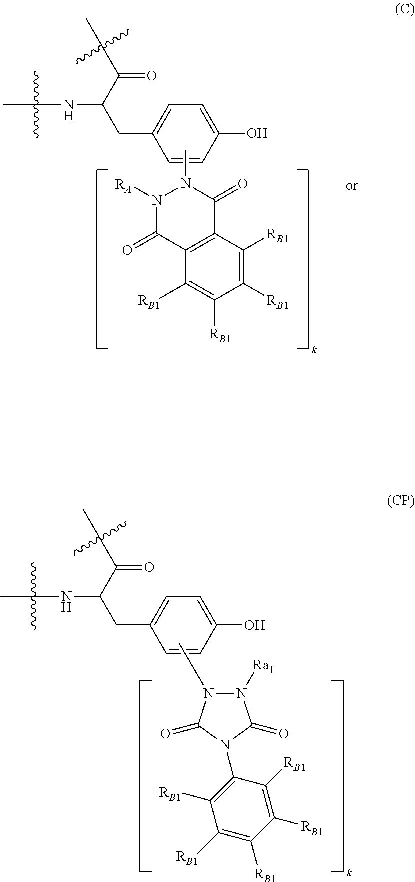
or formula (C-1) or (C-2) as follows: 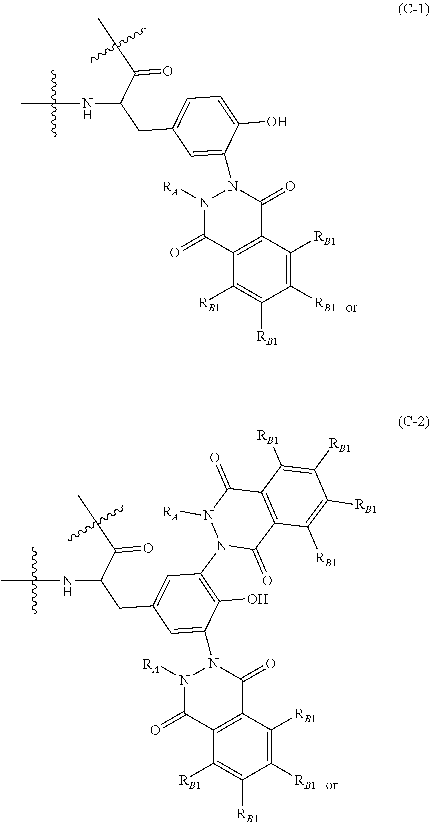
wherein:
k is 1 or 2,
R_{A} is C₁-C₃ alkyl, a phenyl optionally substituted, a benzyl optionally substituted or -(Y)ₙ-M,
Rₐ₁ is R_{A} or H,
each R_{B1} is independently a group of formula -(Y)ₙ-M, a hydrogen or a substituent selected from the group consisting of a halogen, C₁-C₆ alkyl, C₆-C₁₄ aryl, C₃-C₆ cycloalkyl, C₁-C₆ alkoxy, C₁-C₆ alkylamino, C₂-C₆ heterocycle, C₁-C₆ alkanoyl, C₁-C₆ carboxy esters, C₁-C₆ acylamino, —COOH, —CONH₂, —NO₂, —SO₃H, —CN, —CF₃, C₁-C₆ hydroxyalkyl, C₁-C₆ haloalkyl, C₁-C₆ thioalkyl, C₁-C₆ alkylthio, C₂-C₁₀ alkoxyalkyl, and C₂-C₆ alkoxycarbonyloxy,
with proviso that when the chemically modified tyrosine is of formula (C), (C-1) or (C-2), at least one group among R_{A} and R_{B1} groups is -(Y)ₙ-M and when the chemical reagent is of formula (CP), at least one group among Rₐ₁ and Rₐ₁ groups is -(Y)ₙ-M.n is 0 or 1,
Y is a spacer, and
M is a functional moiety.

32. The cellular entity of claim 31, which is characterized by one or several specific features:
all R_{B1} groups are all H except one R_{B1} which is a group of formula -(Y)ₙ-M, and/or
R_{A} is C₁-C₃ alkyl, a phenyl optionally substituted, or a benzyl optionally substituted, and/or
M is selected from the group consisting of a click chemistry reactive group, a cytotoxic or antitumoral drug, a labelling agent, a shielding or masking agent, protein tags, and polypeptides comprising an antigen-binding domain, and/or
the cellular entity is selected from the group consisting of bacteria, erythrocytes, Hematopoietic Stem cells (HSC), Peripheral Blood Mononuclear cells (PBMC), T lymphocytes and dendritic cells (DC).

33. A method of treating or imaging a subject comprising administering the cellular entity of claim 31 to a subject.

34. An in vitro method comprising contacting a cell, tissue or matrix with the cellular entity of claim 31 and examining the interaction of said cell, tissue, or matrix with said cellular entity or imaging the interaction of said cellular entity with a cell, tissue, or matrix.
